SEQUENCE LISTING (134 sequences)

SEQ ID NO: 1 - protein, 704 aa, synthetic construct
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSSSELTQDPAVSVALGQTVRITCQGDSLRSYYGSWYQQKPGQAPVLVIYGRNNRPSGIPDRFSGSSSGNTASLTITGAQAEDEADYYCNSKDSSGNHVVFGGGTKLTVLGGGGSGGGDSGGGGSGGGGSEVQLVQSGGGVERPGGSLRLSCAASGFTFDDYSMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 2 - protein, 711 aa, synthetic construct
QVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSSSELTQDPAVSVALGQTVRITCQGDSLRSYYGSWYQQKPGQAPVLVIYGRNNRPSGIPDRFSGSSSGNTASLTITGAQAEDEADYYCNSKDSSGNHVVFGGGTKLTVLGGGGSGGGDSGGGGSGGGGSEVQLVQSGGGVERPGGSLRLSCAASGFTFDDYSMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 3 - protein, 707 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSSSELTQDPAVSVALGQTVRITCQGDSLRSYYGSWYQQKPGQAPVLVIYGRNNRPSGIPDRFSGSSSGNTASLTITGAQAEDEADYYCNSKDSSGNHVVFGGGTKLTVLGGGGSGGGDSGGGGSGGGGSEVQLVQSGGGVERPGGSLRLSCAASGFTFDDYSMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 4 - protein, 711 aa, synthetic construct
QVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGDSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 5 - protein, 707 aa, synthetic construct
QVQLQESGPGLVKPSQTLSLTCTVSGGSISSGDYFWSWIRQLPGKGLEWIGHIHNSGTTYYNPSLKSRVTISVDTSKKQFSLRLSSVTAADTAVYYCARDRGGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGDSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 6 - protein, 704 aa, synthetic construct
EVQLVESGGGLVQPGGSLRLSCAASGFTFSSYVMSWVRQAPGKGLEWVATISSGGSYTYYPDSVKGRFTISRDNAKNTLYLQMNSLRAEDTAVYYCARRGDSMITTDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGDSGGGGSGGGGSEVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 7 - protein, 18 aa, Homo sapiens
NDNSAEMCRKCSTGCPRG

SEQ ID NO: 8 - protein, 18 aa, Homo sapiens
EEDSPEMCRKCRTGCPRG

SEQ ID NO: 9 - protein, 18 aa, Pan troglodytes
EEDSPEMCRKCRTRCPRG

SEQ ID NO: 10 - protein, 18 aa, unidentified
EEDSPEICRKCRTGCPRG

SEQ ID NO: 11 - protein, 18 aa, Homo sapiens
EEDSPEMCRKCYTGCPRG

SEQ ID NO: 12 - protein, 18 aa, Homo sapiens
EEDSPEMCEECETGCPRG

SEQ ID NO: 13 - protein, 19 aa, Homo sapiens
EEDSPEMCRKCSRTGCPRG

SEQ ID NO: 14 - protein, 19 aa, Homo sapiens
EEDSPEMCRKCARTGCPRG

SEQ ID NO: 15 - protein, 121 aa, synthetic construct
EVQLVQSGGGVERPGGSLRLSCAASGFTFKDYSMSWVRQAPGKGLEWVSGINWNSMKTAYADSVKGRVTISRDNAKNSLYLQMNSLRAEDTAVYYCAKITGAGRGWYFDAWGKGTTVTVSS

SEQ ID NO: 16 - protein, 8 aa, synthetic construct
GFTFKDYS

SEQ ID NO: 17 - protein, 9 aa, synthetic construct
INWNSMKTA

SEQ ID NO: 18 - protein, 14 aa, synthetic construct
AKITGAGRGWYFDA

SEQ ID NO: 19 - protein, 119 aa, synthetic construct
EVQLVQSGGGVERPGGSLRLSCAASGFTFDDYSMSWVRQAPGKGLEWVSGINWNLRKTAYADSVKGRVTISRDNAKNSLYQMNSLRAEDTAVYYCAKILGAGRGWYDAWGKGTTVTVSS

SEQ ID NO: 20 - protein, 8 aa, synthetic construct
GFTFDDYS

SEQ ID NO: 21 - protein, 9 aa, synthetic construct
INWNLRKTA

SEQ ID NO: 22 - protein, 14 aa, synthetic construct
AKILGAGRGWYFDA

SEQ ID NO: 23 - protein, 120 aa, synthetic construct
EVQLVQSGGGVERPGGSLRLSCAASGFTFDDYSMSWVRQAPGKGLEWVSGINWNLRKTAYADSVKGRVTISRDNAKNSLYLQMNSLRAEDTAVYYCAKILGAGRGWYFDWGKGTTVTVSS

SEQ ID NO: 24 - protein, 8 aa, synthetic construct
GFTFNFTS

SEQ ID NO: 25 - protein, 9 aa, synthetic construct
INWNGGSTA

SEQ ID NO: 26 - protein, 121 aa, synthetic construct
EVQLVQSGGGVERPGGSLRLSCAASGFTFDDYSMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 27 - protein, 108 aa, synthetic construct
SSELTQDPAVSVALGQTVRITCQGDSLRSYYGSWYQQKPGQAPVLVIYGRNNRPSGIPDRFSGSSSGNTASLTITGAQAEDEADYYCNSKDSSGNHVVFGGGTKLTVL

SEQ ID NO: 28 - protein, 9 aa, synthetic construct
INWNGGSRR

SEQ ID NO: 29 - protein, 121 aa, synthetic construct
EVQLVQSGGGVERPGGSLRLSCAASGFTFDDYAMSWVRQAPGKGLEWVSGINWNGGSRRYADSVKGRVTISRDNAKNSLYLQMNSLRAEDTAVYYCAKILGAGRGWYFYYWGKGTTYTYSS

SEQ ID NO: 30 - protein, 8 aa, synthetic construct
GFTFDDYA

SEQ ID NO: 31 - protein, 14 aa, synthetic construct
AKILGAGRGWYFYY

SEQ ID NO: 32 - protein, 121 aa, synthetic construct
EVQLVQSGGGVERPGGSLRLSCAASGFTFDDYWMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 33 - protein, 8 aa, synthetic construct
GFTFDDYW

SEQ ID NO: 34 - protein, 121 aa, synthetic construct
EVQLVQSGGGVERPGGSLRLSCAASGFTFDDYRMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 35 - protein, 8 aa, synthetic construct
GFTFDDYY

SEQ ID NO: 36 - protein, 8 aa, synthetic construct
GFTFDDYR

SEQ ID NO: 37 - protein, 121 aa, synthetic construct
EVQLVQSGGGVERPGGSLRLSCAASGFTFDDYAMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 38 - protein, 121 aa, synthetic construct
EVQLVQSGGGVERPGGSLRLSCAASGFTFDDYKMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 39 - protein, 8 aa, synthetic construct
GFTFDDYK

SEQ ID NO: 40 - protein, 107 aa, synthetic construct
SSELTQDPAVSVALGQTVRITCQGDNNQSTYGSWYQQKPGQAPVLVIYGYNNRPSGIPDRFSGSSSGNTASLTITGAQAEDEADYYCNSKFKGNRSVFGGGTKLTVL

SEQ ID NO: 41 - protein, 8 aa, synthetic construct
NNQSTYGS

SEQ ID NO: 42 - protein, 4 aa, synthetic construct
GYNN

SEQ ID NO: 43 - protein, 10 aa, synthetic construct
NSKFKGNRSV

SEQ ID NO: 44 - protein, 107 aa, synthetic construct
SSELTQDPAVSVALGQTVRITCQGDGLGSYTGSWYQQKPGQAPVLVIYSRNNRPSGIPDRFSGSSSGNTASLTITGAQAEDEADYYCTLDSSMNHGVFGGGTKLTVL

SEQ ID NO: 45 - protein, 8 aa, synthetic construct
GLGSYTGS

SEQ ID NO: 46 - protein, 4 aa, synthetic construct
SRNN

SEQ ID NO: 47 - protein, 10 aa, synthetic construct
TLDSSMNHGV

SEQ ID NO: 48 - protein, 11 aa, synthetic construct
NSKDSSGNHVV

SEQ ID NO: 49 - protein, 108 aa, synthetic construct; identical to SEQ ID NO: 27

SEQ ID NO: 50 - protein, 8 aa, synthetic construct
SLRSYYGS

SEQ ID NO: 51 - protein, 4 aa, synthetic construct
GRNN

SEQ ID NO: 52 - protein, 108 aa, synthetic construct
SSELTQDPAVSVALGQTVRITCQGDSARSYLGSWYQQKPGQAPVLVIYYRNNRPSGIPDRFSGSSSGNTASLTITGAQAEDEADYYCNSKCSYGNHVVFGGGTKLTVL

SEQ ID NO: 53 - protein, 8 aa, synthetic construct
SARSYLGS

SEQ ID NO: 54 - protein, 4 aa, synthetic construct
YRNN

SEQ ID NO: 55 - protein, 11 aa, synthetic construct
NSKCSYGNHVV

SEQ ID NO: 56 - protein, 108 aa, synthetic construct
SSELTQDPAVSVALGQTVRITCQGDFARSYLGSWYQQKPGQAPVLVIYYRGSRPSGIPDRFSGSSSGNTASLTITGAQAEDEADYYCNKKCSYGLHAVFGGGTKLTVL

SEQ ID NO: 57 - protein, 8 aa, synthetic construct
FARSYLGS

SEQ ID NO: 58 - protein, 4 aa, synthetic construct
YRGS

SEQ ID NO: 59 - protein, 11 aa, synthetic construct
NKKCSYGLHAV

SEQ ID NO: 60 - protein, 121 aa, synthetic construct
EVQLVQSGGGVERPGGSLRLSCAASGFTFDDYSMSWVRQAPGKGLEWVSGINWNGGSTAYAKSVKGRVTISRDNAKNSLYLQMNSLRAEDTAVYYCAKILGAGRGWYFDAWGKGTTVTVSS

SEQ ID NO: 61 - protein, 109 aa, synthetic construct
SSELTQDPAVSVALGQTVRITCQGDSLRSYYGSWYQQKPGQAPVLVIYGRNNRPSGIPDRFSGSSSGNTASLTITGAQAEDEADYYCNSKDSSGNHVVYFGGGTKLTVL

SEQ ID NO: 62 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 63 - protein, 411 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 64 - protein, 5 aa, synthetic construct
ISEDG

SEQ ID NO: 65 - protein, 4 aa, synthetic construct
SEDG

SEQ ID NO: 66 - protein, 4 aa, synthetic construct
SAAA

SEQ ID NO: 67 - protein, 4 aa, synthetic construct
KYGQ

SEQ ID NO: 68 - protein, 4 aa, synthetic construct
AAGA

SEQ ID NO: 69 - protein, 10 aa, synthetic construct
HWNDLLFCLR

SEQ ID NO: 70 - protein, 7 aa, synthetic construct
GEVELSP

SEQ ID NO: 71 - protein, 4 aa, synthetic construct
VELS

SEQ ID NO: 72 - protein, 4 aa, synthetic construct
VALA

SEQ ID NO: 73 - protein, 10 aa, synthetic construct
DSPEMCRKCR

SEQ ID NO: 74 - protein, 4 aa, synthetic construct
EMCR

SEQ ID NO: 75 - protein, 4 aa, synthetic construct
AACR

SEQ ID NO: 76 - protein, 8 aa, synthetic construct
TKHSGEVP

SEQ ID NO: 77 - protein, 18 aa, synthetic construct
EEDSPEMCRKCWTGCPRG

SEQ ID NO: 78 - protein, 4 aa, synthetic construct
RKCW

SEQ ID NO: 79 - protein, 4 aa, synthetic construct
RKCY

SEQ ID NO: 80 - protein, 5 aa, synthetic construct
RKCAR

SEQ ID NO: 81 - protein, 5 aa, synthetic construct
RKCSR

SEQ ID NO: 82 - protein, 4 aa, synthetic construct
EECE

SEQ ID NO: 83 - protein, 77 aa, synthetic construct
SGDSLRSYYASWYQQKPGQAPVLVIYGANNRPSGIPDRFSGSSSGNTASLTITGAQAEDEADYYCNSADSSGNHVVF

SEQ ID NO: 84 - protein, 87 aa, synthetic construct
SGFTFDDYAMSWVRQAPGKGLEWVSGINWQGGSTGYADSVKGRVTISRDNAKNSLYLQMNSLRAEDTAVYYCAKILGAGRGWYFDYW

SEQ ID NO: 85 - protein, 121 aa, synthetic construct; identical to SEQ ID NO: 15

SEQ ID NO: 86 - protein, 121 aa, synthetic construct
EVQLVQSGGGVERPGGSLRLSCAASGFTFDDYSMSWVRQAPGKGLEWVSGINWNLRKTAYADSVKGRVTISRDNAKNSLYLQMNSLRAEDTAVYYCAKILGAGRGWYFDAWGKGTTVTVSS

SEQ ID NO: 87 - protein, 121 aa, synthetic construct
EVQLVQSGGGVERPGGSLRLSCAASGFTFNFTSMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 88 - protein, 121 aa, synthetic construct; identical to SEQ ID NO: 26

SEQ ID NO: 89 - protein, 121 aa, synthetic construct
EVQLVQSGGGVERPGGSLRLSCAASGFTFDDYAMSWVRQAPGKGLEWVSGINWNGGSRRYADSVKGRVTISRDNAKNSLYLQMNSLRAEDTAVYYCAKILGAGRGWYFYYWGKGTTVTVSS

SEQ ID NO: 90 - protein, 121 aa, synthetic construct; identical to SEQ ID NO: 32

SEQ ID NO: 91 - protein, 121 aa, synthetic construct
EVQLVQSGGGVERPGGSLRLSCAASGFTFDDYYMSWVRQAPGKGLEWVSGINWNGGSTAYADSVKGRVTISRDNAKNSLYLQMNSLRAEDTAVYYCAKILGAGRGWYFDAWGKGTTVTVSS

SEQ ID NO: 92 - protein, 121 aa, synthetic construct; identical to SEQ ID NO: 34

SEQ ID NO: 93 - protein, 121 aa, synthetic construct; identical to SEQ ID NO: 37

SEQ ID NO: 94 - protein, 121 aa, synthetic construct; identical to SEQ ID NO: 38

SEQ ID NO: 95 - protein, 107 aa, synthetic construct; identical to SEQ ID NO: 40

SEQ ID NO: 96 - protein, 107 aa, synthetic construct; identical to SEQ ID NO: 44

SEQ ID NO: 97 - protein, 107 aa, synthetic construct; identical to SEQ ID NO: 44

SEQ ID NO: 98 - protein, 108 aa, synthetic construct; identical to SEQ ID NO: 27

SEQ ID NO: 99 - protein, 108 aa, synthetic construct; identical to SEQ ID NO: 52

SEQ ID NO: 100 - protein, 108 aa, synthetic construct; identical to SEQ ID NO: 56

SEQ ID NO: 101 - protein, 4 aa, synthetic construct
RKCR

SEQ ID NO: 102 - protein, 4 aa, synthetic construct
RKAR

SEQ ID NO: 103 - protein, 4 aa, synthetic construct
AAAA

SEQ ID NO: 104 - protein, 4 aa, synthetic construct
EECR

SEQ ID NO: 105 - protein, 5 aa, synthetic construct
CRKCR

SEQ ID NO: 106 - protein, 5 aa, synthetic construct
CAACA

SEQ ID NO: 107 - protein, 5 aa, synthetic construct
CEECE

SEQ ID NO: 108 - protein, 5 aa, synthetic construct
CYYCR

SEQ ID NO: 109 - protein, 5 aa, synthetic construct
CLLCR

SEQ ID NO: 110 - protein, 5 aa, synthetic construct
CWWCR

SEQ ID NO: 111 - protein, 5 aa, synthetic construct
CAACR

SEQ ID NO: 112 - protein, 5 aa, synthetic construct
CNNCR

SEQ ID NO: 113 - protein, 5 aa, synthetic construct
CTTCR

SEQ ID NO: 114 - protein, 5 aa, synthetic construct
CSSCR

SEQ ID NO: 115 - protein, 5 aa, synthetic construct
CQQCR

SEQ ID NO: 116 - protein, 5 aa, synthetic construct
CEECR

SEQ ID NO: 117 - protein, 5 aa, synthetic construct
CDDCR

SEQ ID NO: 118 - protein, 5 aa, synthetic construct
CKKCR

SEQ ID NO: 119 - protein, 4 aa, synthetic construct
YYCR

SEQ ID NO: 120 - protein, 4 aa, synthetic construct
LLCR

SEQ ID NO: 121 - protein, 4 aa, synthetic construct
WWCR

SEQ ID NO: 122 - protein, 4 aa, synthetic construct
NNCR

SEQ ID NO: 123 - protein, 4 aa, synthetic construct
TTCR

SEQ ID NO: 124 - protein, 4 aa, synthetic construct
SSCR

SEQ ID NO: 125 - protein, 4 aa, synthetic construct
QQCR

SEQ ID NO: 126 - protein, 4 aa, synthetic construct
DDCR

SEQ ID NO: 127 - protein, 4 aa, synthetic construct
KKCR

SEQ ID NO: 128 - protein, 40 aa, Homo sapiens
QKRSSPSEGLCPPGHHISEDGRDCISCKYGQDYSTHWNDL

SEQ ID NO: 129 - protein, 40 aa, Homo sapiens
LFCLRCTRCDSGEVELSPCTTTRNTVCQCEEGTFREEDSP

SEQ ID NO: 130 - protein, 31 aa, Homo sapiens
EMCRKCRTGCPRGMVKVGDCTPWSDIECVHK

SEQ ID NO: 131 - protein, 4 aa, synthetic construct
KKIK

SEQ ID NO: 132 - protein, 77 aa, synthetic construct
QGDSLRSYYASWYQQKPGQAPVLVIYGKNNRPSGIPDRFSGSSSGNTASLTITGAQAEDEADYYCNSRDSSGNHVVF

SEQ ID NO: 133 - protein, 87 aa, synthetic construct
SGFTFDDYGMSWVRQAPGKGLEWVSGINWNGGSTGYADSVKGRVTISRDNAKNSLYLQMNSLRAEDTAVYYCAKILGAGRGWYFDLW

SEQ ID NO: 134 - protein, 4 aa, synthetic construct
AACA

(19) United States
(12) Patent Application Publication
(10) Pub. No.: US 2025/0376532 A1
(43) Pub. Date: Dec. 11, 2025

(54) STRATEGY FOR HIGHLY SUPERIOR DR5 ACTIVATION INCLUDING IN TUMORS AND CANCERS

(71) Applicant: University of Virginia Patent Foundation, Charlottesville, VA (US)

(72) Inventors: Jogender Tushir-Singh, Davis, CA (US); Sanchita Bhatnagar, Davis, CA (US)

(73) Assignee: University of Virginia Patent Foundation, Charlottesville, VA (US)

(21) Appl. No.: 18/685,191

(22) PCT Filed: Aug. 22, 2022
(86) PCT No.: PCT/US2022/075301

§ 371 Date: Feb. 20, 2024

Related U.S. Application Data
(60) Provisional application No. 63/235,445, filed on Aug. 20, 2021.

(51) Int. Cl. C07K 16/28; A61K 39/00; A61P 35/00

(52) U.S. Cl. CPC C07K 16/2878; A61P 35/00; A61K 2039/505; C07K 2317/31; C07K 2317/35; C07K 2317/73; C07K 2317/75; C07K 2317/92

(57) ABSTRACT

Provided are methods for treating cancers in subjects in need thereof. In some embodiments, the methods include administering to a subject in need thereof a first composition that includes an effective amount of a binding agent that selectively binds to a tetrapeptide motif of a human CRD3 of a DR5 polypeptide and a second composition that includes an effective amount of a DR5 agonist. Also provided are methods for activating DR5 biological activities in cells, tissues, and organs, optionally cells, tissues, and organs present in subject; and compositions for use in the presently disclosed methods, including but not limited to compositions that include an effective amount of a binding agent that selectively binds to the tetrapeptide motif RKCR (SEQ ID NO: 101) of a human CRD3 of DR5 and an effective amount of a DR5 agonist and antibodies that bind to a death receptor 5 (DR5) polypeptide, including but not limited to bispecific antibodies.

### CROSS REFERENCE TO RELATED APPLICATION

The presently disclosed subject matter claims the benefit of U.S. Provisional Patent Application Ser. No. 63/235,445, filed Aug. 20, 2021, the disclosure of which incorporated herein by reference in its entirety.

### GRANT STATEMENT

This invention was made with government support under Grant No. CA233752 awarded by the National Institutes of Health and under Grant Nos. W81XWH-18-1-0048 and W81XWH-19-1-0190 awarded by the Department of Defense. The government has certain rights in the invention.

### REFERENCE TO SEQUENCE LISTING XML

The Sequence Listing XML associated with the instant disclosure has been electronically submitted to the United States Patent and Trademark Office via the Patent Center as a 125,575 byte UTF-8-encoded XML file created on Aug. 22, 2022 and entitled “3062_163_PCT.xml”. The Sequence Listing submitted via Patent Center is hereby incorporated by reference in its entirety.

### TECHNICAL FIELD

The presently disclosed subject matter relates to cellular apoptosis. In particular, the presently disclosed subject matter relates to Death Receptor 5 (DR5) activation, potential treatments of cancer and compositions thereof.

### BACKGROUND

Death receptor-5 (DR5) belongs to the TNF-α superfamily. The polypeptide includes three external cysteine-rich domains (CRDs 1-3) and activates apoptotic signaling outside the cells (Ashkenazi & Herbst, 2008). The DR5 ligand Apo2L (also called TNF superfamily member 10 (TNFSF10), cluster of differentiation 253 (CD253), and TNF-related apoptosis-inducing ligand (TRAIL)) and agonist strategies orchestrate apoptotic cytotoxicity via assembling an activated death-inducing signaling complex (DISC) under the lipid-bilayer, a process that requires higher-ordered clustering of DR5 receptors (Hymowitz et al., 1999). However, the mechanism of external DR5 clustering initiation and maintenance remains highly elusive till today. Multiple DR5 agonist antibodies such as lexatumumab (also referred to herein as “LEXA” or “Lexa”; a humanized HGS-ETR2 monoclonal antibody resulting from a collaboration between Human Genome Sciences, Rockville, Maryland, United States of America and Cambridge Antibody Technology, Cambridgeshire, England, United Kingdom; also called ETR2-ST01; Johnson et al., 2003), AMG655 (Conatumumab; Amgen Inc., Thousand Oaks, California, United States of America; Kaplan-Lefko et al., 2010), KMTR2 (Kyowa Kirin Co., Ltd., Tokyo, Japan; Motoki et al., 2005), tigatuzumab (a humanized version of TRA-8 from Daiichi Sankyo Co., Ltd., Tokyo, Japan; Forero-Torres et al., 2010a), and apomab (Genentech, Inc., South San Francisco, United States of America; Adams et al., 2008a), have been tested in clinics for various solid cancer indications (Ashkenazi & Herbst, 2008; Shivange et al., 2018; Wajant, 2019). Unfortunately, all DR5 agonists to date have failed in Phase II clinical trials due to limited apoptotic cytotoxicity induction owing to limited receptor clustering and maintenance (Ashkenazi, 2015; Forero-Torres et al., 2010b).

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter relates to methods for treating cancers in subjects in need thereof. In some embodiments, the methods comprise, consist essentially of, or consist of administering to a subject (a) a first composition comprising an effective amount of a first binding agent that selectively binds to a human cysteine-rich domain 3 (CRD3) of DR5, optionally an RKCR (SEQ ID NO: 101) peptide motif present in a human cysteine-rich domain 3 (CRD3) of DR5, further optionally a DR5 comprising an amino acid sequence as set forth in SEQ ID NO: 62 or SEQ ID NO: 63); and (b) a second composition comprising, consisting essentially of, or consisting of an effective amount of a second binding agent, wherein the second binding agent selectively binds to a binding partner selected from the group consisting of a human cysteine-rich domain 1 (CRD1), a cysteine-rich domain 2 (CRD2), and a tumor-associated antigen, optionally wherein the second binding agent comprises a DR5 agonist. In some embodiments, the cancer comprises a solid tumor. In some embodiments, the first binding agent, the second binding agent, or both comprise an antibody. In some embodiments, the antibody is selected from the group consisting of lexatumumab and antibody 1114, wherein antibody 1114 comprises a heavy chain comprising an amino acid sequence as set forth in FIG. 5A, in some embodiments an amino acid sequence as set forth in any one of SEQ ID NOs: 85-94, a light chain comprising an amino acid sequence as set forth in FIG. 5B, in some embodiments an amino acid sequence as set forth in any one of SEQ ID NOs: 95-100, a biologically active fragment thereof, a homolog thereof, or any combination thereof.

In some embodiments of the presently disclosed methods, the second binding agent selectively binds to at least one of a CRD1 and a CRD2 of DR5. In some embodiments, the second binding agent has DR5 agonist activity. In some embodiments, the second binding agent comprises an antibody, optionally an antibody with DR5 agonist activity. In some embodiments, the first binding agent has DR5 agonist activity.

In some embodiments, the first composition and the second composition are provided as a single composition. In some embodiments, the single composition comprises a bispecific antibody. In some embodiments, the single composition comprises a 2DEI antibody. In some embodiments, the 2DEI antibody comprises an amino acid sequence as set forth in Table 3. In some embodiments, the 2DEI antibody comprises an amino acid sequence as set forth in any one of SEQ ID NOs: 1-6, or a biologically active fragment and/or homolog thereof. In some embodiments, the single composition comprises an antibody selected from the group comprising lexatumumab and antibody 1114, wherein antibody 1114 comprises a heavy chain comprising an amino acid sequence as set forth in FIG. 5A, in some embodiments any one of SEQ ID NOs: 85-94, a light chain comprising an amino acid sequence as set forth in FIG. 5B, in some embodiments any one of SEQ ID NOs: 95-100, a biologically active fragment thereof, a homolog thereof, or any combination thereof.

The presently disclosed subject matter also relates in some embodiments to compositions comprising (a) an effective amount of a first binding agent that selectively binds to a human cysteine-rich domain 3 (CRD3) of DR5, in some embodiments that selectively binds an RKCR (SEQ ID NO: 101) peptide motif present in a human cysteine-rich domain 3 (CRD3) of DR5, in some embodiments a DR5 comprising an amino acid sequence as set forth in SEQ ID NO: 62 or SEQ ID NO: 63); and (b) an effective amount of a second binding agent that selectively binds to a human cysteine-rich domain 1 (CRD1), a cysteine-rich domain 2 (CRD2), or a tumor-associated antigen, In some embodiments, the second binding agent has DR5 agonist activity. In some embodiments, the first binding agent has DR5 agonist activity. In some embodiments, the composition comprises a bispecific antibody. In some embodiments, the composition comprises a 2DEI antibody, optionally wherein the 2DEI antibody comprises an amino acid sequence as set forth in Table 3, in some embodiments an amino acid sequence as set forth in any one of SEQ ID NOs: 1-6, or a biologically active fragment or homolog thereof.

In some embodiments, the presently disclosed compositions further comprise a pharmaceutically acceptable carrier, which in some embodiments can be a pharmaceutically acceptable carrier that is pharmaceutically acceptable for use in a human.

The presently disclosed subject matter also relates in some embodiments to bispecific antibodies that bind to a death receptor 5 (DR5) polypeptide. In some embodiments, the bispecific antibody comprises a first antigen binding moiety that is specific for an RKCR (SEQ ID NO: 101) tetrapeptide motif of a human CRD3 of DR5 and a second antigen binding moiety that is specific for an epitope of DR5 that is distinct from the RKCR (SEQ ID NO: 101) tetrapeptide motif or a tumor-associated antigen. In some embodiments, the second antigen binding moiety has DR5 agonist activity. In some embodiments, the first binding moeity or the second binding moeity is an antibody selected from the group consisting of AMG655, KMTR2, Tigatuzumab, lexatumumab, apomab, and antibody 1114, wherein antibody 1114 selectively binds to an RKCR (SEQ ID NO: 101) tetrapeptide motif present in a human cysteine-rich domain 3 (CRD3) of DR5, optionally a DR5 comprising an amino acid sequence as set forth in SEQ ID NO: 62 or SEQ ID NO: 63, or is a biologically active fragment or homolog thereof. In some embodiments, the first binding moiety or the second binding moiety comprises a 2DEI antibody and/or a biologically active fragment or derivative thereof, optionally wherein the 2DEI antibody and/or the biologically active fragment or derivative thereof comprises an amino acid sequence as set forth in Table 3, optionally an amino acid sequence as set forth in any one of SEQ ID NOs: 1-6. In some embodiments, the antibody is humanized. In some embodiments, a bispecific antibody of the presently disclosed subject matter further comprises a pharmaceutically acceptable carrier, optionally a pharmaceutically acceptable carrier that is pharmaceutically acceptable for use in a human.

the presently disclosed subject matter also provides in some embodiments antibodies that bind to a death receptor 5 (DR5) polypeptide, wherein the antibody comprises an antigen binding site that binds to a human CRD3, optionally to an RKCR (SEQ ID NO: 101) tetrapeptide motif of a human CRD3 of DR5, further optionally wherein the antibody is antibody 1114, wherein antibody 1114 comprises a heavy chain comprising an amino acid sequence as set forth in SEQ ID NO: 26 and a light chain comprising an amino acid sequence as set forth in SEQ ID NO: 27, a biologically active fragment thereof, a homolog thereof, or any combination thereof. In some embodiments, the antibody is humanized. In some embodiments, the antibody further comprises a pharmaceutically acceptable carrier, optionally a pharmaceutically acceptable carrier that is pharmaceutically acceptable for use in a human. In some embodiments, the antibody is a bispecific antibody, optionally a bispecific antibody that comprises a binding arm that binds to a tumor-associated antigen.

The presently disclosed subject matter also provides in some embodiments methods for activating DR5 biological activities in cells, tissues, and/or organs, optionally cells, tissues, and/or organs present in a subject. In some embodiments, the methods comprise, consist essentially of, or consist of administering to the subject (a) a first composition comprising an effective amount of a first binding agent that selectively binds to a human cysteine-rich domain 3 (CRD3) of DR5, optionally an RKCR (SEQ ID NO: 101) peptide motif present in a human cysteine-rich domain 3 (CRD3) of DR5, further optionally a DR5 comprising an amino acid sequence as set forth in SEQ ID NO: 62 or SEQ ID NO: 63; and (b) a second composition comprising, consisting essentially of, or consisting of an effective amount of a second binding agent, wherein the second binding agent selectively binds to a binding partner selected from the group consisting of a human cysteine-rich domain 1 (CRD1), a cysteine-rich domain 2 (CRD2), and a tumor-associated antigen, optionally wherein the second binding agent comprises a DR5 agonist. In some embodiments, the DR5 biological activity to be activated is present in a cell of a tumor or a cancer, optionally a cell of a solid tumor. In some embodiments, the first binding agent comprises an antibody, optionally an antibody that is selected from the group consisting of lexatumumab and antibody 1114, wherein antibody 1114 comprises a heavy chain comprising an amino acid sequence as set forth in SEQ ID NO: 26 and a light chain comprising an amino acid sequence as set forth in SEQ ID NO: 27, or a biologically active fragment or homolog thereof, and further wherein antibody 1114 selectively binds an RKCR (SEQ ID NO: 101) tetrapeptide motif present in a human cysteine-rich domain 3 (CRD3) of DR5, optionally a DR5 comprising an amino acid sequence as set forth in SEQ ID NO: 62 or SEQ ID NO: 63. In some embodiments, the second binding agent that selectively binds to a human CRD1 and/or a human CRD2, optionally wherein the second binding agent comprises a DR5 agonist. In some embodiments, the DR5 agonist comprises an antibody, optionally an antibody that binds to DR5 CRD1 and/or CRD2, or to an epitope other than a RKCR (SEQ ID NO: 101) tetrapeptide of DR5. In some embodiments, the first composition and the second composition are provided as a single composition. In some embodiments, the single composition comprises a bispecific antibody, optionally a 2DEI antibody. In some embodiments, the single composition comprises a 2DEI antibody, optionally wherein the 2DEI antibody comprises a sequence as set forth in Table 3, optionally any one of SEQ ID NOs: 1-6, or a biologically active fragment and/or homolog thereof. In some embodiments, the single composition comprises an antibody selected from the group comprising lexatumumab and antibody 1114, wherein antibody 1114 comprises a heavy chain comprising an amino acid sequence as set forth in SEQ ID NO: 26 and a light chain comprising an amino acid sequence as set forth in SEQ ID NO: 27, a biologically active fragment thereof, a homolog thereof, or any combination thereof.

In some embodiments, the presently disclosed subject matter also relates to uses of the presently disclosed compositions, the presently disclosed bispecific antibodies, and/or the presently disclosed antibodies for treating cancers in subjects in need thereof and/or for activating DR5 biological activities in cells, tissues, and/or organs.

In some embodiments, the presently disclosed subject matter also relates to compositions for use in treating cancers in subjects in need thereof and/or for activating DR5 biological activities in cells, tissues, and/or organs. In some embodiments, the compositions comprise (a) an effective amount of a binding agent that selectively binds an RKCR (SEQ ID NO: 101) peptide motif present in a human cysteine-rich domain 3 (CRD3) of DR5, optionally a DR5 comprising an amino acid sequence as set forth in SEQ ID NO: 62 or SEQ ID NO: 63; and (b) an effective amount of a DR5 agonist, wherein the DR5 agonist comprises a moiety that binds to a binding partner selected from the group consisting of a human cysteine-rich domain 1 (CRD1), a cysteine-rich domain 2 (CRD2), and a tumor-associated antigen. In some embodiments, the composition comprises a bispecific antibody. In some embodiments, the bispecific antibody comprises a first antigen binding moiety that is specific for an RKCR (SEQ ID NO: 101) tetrapeptide motif present in a human cysteine-rich domain 3 (CRD3) of DR5, optionally a DR5 comprising an amino acid sequence as set forth in SEQ ID NO: 62 or SEQ ID NO: 63, and a second antigen binding moiety that is specific for an epitope of DR5 that is distinct from the RKCR (SEQ ID NO: 101) tetrapeptide motif, optionally an epitope within CRD1 or CRD2 of DR5, and that is a DR5 agonist. In some embodiments, the first binding moeity or the second binding moeity is an antibody selected from the group comprising AMG655, KMTR2, Tigatuzumab, lexatumumab, apomab, and antibody 1114, wherein antibody 1114 comprises a heavy chain comprising an amino acid sequence as set forth in FIG. 5A, optionally any one of SEQ ID NOs: 85-94, a light chain comprising an amino acid sequence as set forth in FIG. 5B, optionally any one of SEQ ID NOs: 95-100, or is a biologically active fragment or homolog thereof. In some embodiments, the first binding moiety or the second binding moiety comprises a 2DEI antibody and/or a biologically active fragment or derivative thereof, optionally wherein the 2DEI antibody and/or the biologically active fragment or derivative thereof comprises an amino acid sequence as set forth in Table 3, further optionally wherein the amino acid sequence is one of SEQ ID NOs: 1-6. In some embodiments, the bispecific antibody is humanized. In some embodiments, the composition comprises a 2DEI antibody, optionally wherein the 2DEI antibody comprises a sequence as set forth in Table 3, optionally one of SEQ ID NOs: 1-6, or a biologically active fragment or homolog thereof. In some embodiments, the composition further comprises a pharmaceutically acceptable carrier, optionally a pharmaceutically acceptable carrier that is pharmaceutically acceptable for use in a human.

Accordingly, it is an object of the presently disclosed subject matter to provide methods, compositions, and pharmaceuticals for treating cancer and/or activating DR5 biological activities in cells, tissues, and/or organs.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTIONS OF THE FIGURES

FIGS. 1A-1H. Lexatumumab limitedly engages DR5 on the cell surface. (FIG. 1A) The binding kinetics of immobilized biotinylated rDR5-IgG4 against indicated DR5 antibodies were measured using bio-layer interferometry (BLI). When shown in color, blue represents KMTR2, green represents AMG655, purple represents Tiga and red represents Lexa. (FIG. 1B) Cell viability assays of indicated DR5 agonists against OVCAR-3 cells (n=3). When shown in color, a white circle outlined in black represents IgG isotype, a black asterisks represents Farletzumab, a black circle represents Apo2L, a purple circle represents Tiga, a green circle represents AMG655, a red circle represents Lexa and a blue circle represents KMTR2. (FIG. 1C) Tumor cells were treated with indicated DR5 agonists for indicated times. Lysates were analyzed for indicated apoptotic regulators using immunoblotting. (FIG. 1D) The percentage of indicated DR5 agonists bound on tumors cell surface (n=3). (FIG. 1E) Same as FIG. 1D except ±crosslinking on ice with PFA (n=3). (FIG. 1F) Indicated high (MDA-MB-436) and low (MDA-MB-231) DR5 expressing cells were treated with AMG655 or Lexatumumab (Lexa) followed by native immunoprecipitation using an anti-IgG1-Fc secondary antibody. (FIG. 1G) Schematic of DR5 showing binding epitopes of indicated DR5 antibodies and Apo2L. (FIGS. 1H-1 to 1H-4) The binding kinetics of immobilized biotinylated rDR5 (indicated mutations on top) against various DR5 agonist antibodies were measured using BLI. Error bars in FIGS. 1D and 1E represent SD (n=3). When shown in color, green represents AMG655, blue represents KMTR2, red represents Tigatuzumab and purple represents Lexatumumab.

FIGS. 2A-2O. Lexatumumab binds to PPCR in the highly variable domain of DR5. (FIGS. 2A-2E) Tumor cell lysates after indicated DR5 agonists treatments, concentration, and time were analyzed in non-reducing denaturing clustering assays using DR5 immunoblotting. (FIG. 2F) Schematic of the experiment for which the results are shown in FIG. 2G. (FIG. 2G) Upon indicated treatment, OVCAR-3 cells lysates were immunoprecipitated as indicated, and unbound and total lysates from the latter were analyzed using DR5 immunoblotting. (FIG. 2H) Colored spheres indicate distinct epitopes of indicated DR5 agonists in CRDs. Different shades of gray represent epitopes. Light gray represented the KMTR2 epitope, dark gray represents the AMG epitope, and gray represents the Lexa epitope. When shown in color, blue represented the KMTR2 epitope, green represents the AMG epitope and red represents the Lexa epitope. (FIG. 2I) A ribbon trace backbone of DR5 bound to indicated DR5 agonists. RKxR harboring CRD3 shows the highest variability. When shown in color, green represents DR5: Apomab, blue represents DR5: Apol2L, orange represents DR5: KMTR2, yellow represents DR5: AMG+Apo2L; red represents DR5: BDF1; gray represents DR4: Apo2L and dark blue represents DR5: YSD1. (FIG. 2J) Schematic of native autoinhibited DR5 ECD showing electrostatic interaction of PPCR motif (CRD3) with negatively charged membrane domains and Lexa binding resulting in conformational change and release. (FIG. 2K) The binding kinetics of immobilized biotinylated 3RE-Mut rDR5 against the indicated antibodies as measured using BLI. When shown in color, green represents AMG655, blue represents KMTR2, red represents Tigatuzumab and purple represents Lexatumumab. (FIG. 2L) Mutated (3RE-DR5 (L)) and wild type (i.e., unmodified; WT-3RE (L)) DR5 stable cells were subjected to IP as indicated. (FIG. 2M) Fold change in IC50 values in 3RE-Mut (RE) DR5 stable cells vs. WT DR5 (L) cells after indicated antibodies (n=3). Error bars correspond to ±SD. *: p<0.05 and **: p<0.01. Actual p values are presented above the asterisks. (FIGS. 2N and 2O) DR5 knocked out MDA-MB-231 (FIG. 2N) and HCC-1806 (FIG. 2O) cells stable expressing WT-DR5(L), and DR5-3RE were grafted with orthotopic tumors in the mammary fat pad. Randomly selected tumor-bearing animals were injected intraperitoneally (i.p.) with IgG1 or KMTR2 (100 μg) every third day. Weights of harvested tumors were quantified and presented in FIG. 2O. Error b applicants respectfully submit correspond to +SD.

FIGS. 3A-3M. Genetic construction and proposed working mechanism of the 2DEI antibody. (FIGS. 3A and 3B) Schematic representations of the genetic construction of an exemplary dual-specificity 2DEI antibody where CRD-1 or CRD-2 of the targeting bispecific partner was engineered to enhance Lexa engagement against PPCR motif before and after higher-order DR5 clustering. When shown in color, red represents Lexatumumab and blue represents anti-DR5 CRD 1 or 2 epitopes. (FIG. 3C) Graph of the binding kinetics of two different immobilized biotinylated rDR5 (indicated mutations) against 2DEI measured using BLI. The 3RE-DR5 mutant (to which 1114 or Lexa cannot bind) confirms binding of KMTR2. The LLF-AAA DR5 mutant was spiked in, (to which KMTR2 cannot bind), confirming the binding of 1114. The two different peaks, one after other confirms the binding of 2DEI antibody to two different immobilized biotinylated rDR5 mutants. (FIG. 3D) DR5 (S) shorter form lacking the 22 membrane proximal amino acids and DR5 (L) larger form including all outside DR5 residues including 22 membrane proximal). Expression in various human TNBC cell lines. (FIGS. 3E-1 and 3E-2) Indicated ovarian and triple-negative breast cancer (TNBC) cell-lines were analyzed in cell viability assays with indicated clinical DR5 agonists and 2DEI antibody (KMTR2-Lexa). When shown in color, a white circle represents IgG Control (Ctrl), a black circle represents Apo2L, a green circle represents AMG655, a red circle represents Lexatumumab, a blue circle represents KMTR2 and an asterisk represents 2DEI (FIGS. 3F-1 and 3F-2) Same as FIG. 3E, except additional 2DEI antibodies (generated with AMG-Lexa, Tiga-Lexa) and random bispecific antibodies were tested against highly resistant TNBC cell lines. When shown in color, a white circle represents IgG Ctrl, a blue circle represents TIGA-AMG, a dark red circle represents TIGA-KMTR2, a red asterisks represents TIGA-Lexa, a green asterisks represents AMG-Lexa and a blue asterisks represents KMTR2-LEXA. (FIG. 3G) IC50 values are shown for the antibodies KMTR2-Lexa, AMG-Lexa, Tiga-Lexa, Tiga-AMG, Tiga-KMTR2, KMTR2-AMG and AMG-Tetra. (FIG. 3H) Immunoblotting of PARP and caspase-3 from indicated cell lysates after indicated treatments, (“u.c.” refers to uncleaved and “c” revers to cleaved). (FIG. 3I) Western blot analysis of DR5 clustering profiles after the indicated treatments. (FIG. 3J) Schematic representation of a genetic construction of an exemplary V_{L}-V_{H} linked Y-format IgG1-like monovalent dual-specificity 2DEI antibody. When shown in color, light blue represents the KMTR2/AMG variable light chain (VL), dark blue represents the KMTR2/AMG variable heavy chain (VH), red represents the Lexa VH and pink represents the Lexa VL. The G4S Linker is represented by a green dotted line. (FIG. 3K) A reducing gel of an exemplary 2DEI-Y format antibody. Only the IgG1 control contains a V_{L} chain on gel. (FIG. 3L) Cell killing of HCC1806 cells in the presence of bivalent bispecific, monovalent bispecific and regular bivalent monospecific indicated antibodies. When shown in color, a black asterisks represents IgG1 Ctrl, a blue asterisks represents KMTR2, a red asterisks represents Lexa, a green asterisks represents AMG, a white circle outlined in red represents 2DEI-Y KMTR2/Lexa (KL), a white circle outlined in blue represents 2KEI-Y AMG/Lexa (AL), a red circle represents 2DEI-X-(KL) and a blue circle represents 2DEI-X (AL). (FIG. 3M) The percentages of total surface DR5 were analyzed at indicated time using a combination of commercial and DR5 antibodies as indicated (n=3).

FIGS. 4A-4N. Enhanced anti-tumor function of 2DEI antibody. (FIG. 4A) Mammary fat pad HCC-1806 tumor-bearing NSG mice were i.p. injected with the indicated antibodies for the indicated days. Animals were live imaged and representative images are shown. (FIG. 4B) Intraperitoneal OV90 ovarian tumor-bearing animals were i.p. injected with 2DEI and random bispecific combinations along with KMTR2. All animals were live imaged when 2 out of 3 IgG1 treated animals became moribund. Representative images are shown. When shown in color, the heat map applies as follows, red represents about 25,000 counts, orange/yellow represents about 20,000 counts. green represents about 15,000 counts, teal represents about 10,000 counts, blue represents about 5,000 counts and violet represents ab out 1,000 counts. (FIG. 4C) Schematic showing exemplary procedure for assaying activities of antibodies in Luc+ MDA-MB-231-engrated mice. NSG animals grafted with Luc+ MDA-MB-231 cells to generate primary subcutaneous tumors. When tumors reached ˜700 mm³ (˜28 days), surgeries were performed. After a 2-week recovery post-surgery, animals were injected i.p. with IgG1, KMTR2, or 2DEI (100 μg for each) every third day, and tumor-bearing animals were live imaged. (FIG. 4D) Bar graphs showing the results of the experiments outlined in FIG. 4C. At the indicated days, accumulated luciferase signal (radiant efficiency) from animals after KMTR2 or 2DEI antibody injections are presented in the bar graphs. Error cbars correspond to +SD. (FIG. 4E) Kaplan-Meier plots depicting the survival of the animals described in FIG. 4C (n=6-9 mice). (FIG. 4F) Kaplan-Meier plot depicting the survival (n=6-12) of mice injected intracardially with highly metastatic MDA-MB-231-2B TNBC cells to generate highly aggressive metastatic tumors that spread to the lung and peritoneal cavities within 4-5 weeks. Randomly selected metastatic tumor-bearing animals were treated with the indicated antibodies (100 μg, 2 times a week) and were also live imaged (not shown). (FIGS. 4G and 4H) Detailed amino acid sequences around DR5 PPCR (red when shown in color) are shown in FIG. 4G, and include SEQ ID NOs: 7-11, 77, and 12-14, respectively, from top to bottom. Various generated recombinant IgG4-Fc human DR5s with mutations/insertions (in PPCR residues) and their binding kinetics with respect to lexatumumab were measured using BLI (FIG. 4H). In FIG. 4H, huDR5 is SEQ ID NO: 8, Monkey-DR5 is SEQ ID NO: 10, Chimp-DR5 is SEQ ID NO: 9, RKCW-huDR5 is SEQ ID NO: 77, RKCAR-huDR5 is SEQ ID NO: 14, RKCSR-huDR5 is SEQ ID NO: 13, and EECE-huDR5 is SEQ ID NO: 12. When shown in color, black represents huDr5, blue represents Monkey-DR5, red represents Chimp-DR5, yellow dashed represents RKCW-huDR5, blue dashed represents RKCY-huDR5, green dashed represented RKCAR-huDR5, purple dashed represents RKCSR-huDr5 and brown dashed represents EECE-huDR5. (FIGS. 4I and 4J) Representation of DR5-Apo2L and DR5-Apomap interface near PPCR motif. Molecular interactions are shown with dotted pink lines. When shown in color, cyan represents the DR5 backbone, green represents Apo2L, and green/light blue represents Apomab-V_{H}/V_{L}. Red, blue, and black colors represent highlight positively, negatively, and cysteine residues, respectively. Additional interface residues are shown with indicated colors. (FIG. 4K) Amino acid sequences of Lexa and Apomab V_{H} and V_{L} CDR subsequences (SEQ ID NOs: 133 and 132, respectively) and Apomab V_{H} and V_{L} CDR subsequences (SEQ ID NOs: 84 and 83, respectively) showing amino acid differences at certain positions indicated with asterisks. Note that not all amino acids of SEQ ID NOs: 63, 84, 132, and 133 are depicted. (FIG. 4L) FACS plots of percentages of total surface DR5 was analyzed at indicated time using a combination of commercial and indicated DR5 antibodies±crosslinking with PFA. (FIG. 4M) Binding affinity comparisons of KMTR2, Lexa, and Apomab IgG1 antibodies as determined by ELISA using either WT DR5 or 3RE-DR5. When shown in color, a red circle represents Lexa DR5 (WT), a black circle represents Apomab DR5 (WT), a blue circle represents KMTR2 DR5 (WT), a white circle outlined in red represents Lexa DR5 (3RA), a white circle outlined in black represents Apomab DR5 (3RA) and a black asterisks represents IgG1 Ctrl. (FIG. 4N) Percent survival analyses of HCC-1806 cells in the presence of the 2DEI generated either with Lexa or Apomab along with control antibodies.

FIGS. 5A-5F. New 1114 antibody that binds PPCR and works effectively in 2DEI antibody. (FIGS. 5A and 5B) V_{H} (FIG. 5A; SEQ ID NOs: 85-94) and V_{L} (FIG. 5B, SEQ ID NOs: 95-100) of various DR5 CRD3-binding antibodies of the presently disclosed subject matter. CDR sequences (when shown in color, blue; light gray amino acids corresponding to CDR1, CDR2, and CDR3) of V_{H} (FIG. 5A) and V_{L} (FIG. 5B) were randomly mutated and only antibody clones that expressed were further analyzed for tumor cell killing activity and binding against WT DR5 and 3RE mutant DR5. (FIG. 5C) Percentage cell killing of OVCAR-3 cells with the indicated V_{H}-V_{L} combinations. Only the 1114 antibody killed 100% of ovarian cancer cells. (FIG. 5D) Amino acid sequences of 1114 V_{H} and V_{L} (corresponding to SEQ ID NOs: 26 and 27, respectively). (FIG. 5E) ELISA data of 1114 against WT and 3RE mutant (PPCR mutant) DR5. When shown in color, a blue circle represents DR5-RKCR-RKAR, a red square represents DR5-RKCR-AAAA, a green triangle represents DR5-AACR, an upside down olive triangle represents 1114 WT DR5 (S) and an upside down white triangle outlined in black represents 1114-WT DR5 (L). (FIG. 5F) Percentage cell killing of HCC 1806 cells treated with the indicated antibodies, including KMTR2-1114, a 2DEI antibody engineered with KMTR2 and 1114. Error bars are ±SD.

FIGS. 6A-6J. Lexatumumab analysis of DR5. (FIGS. 6A-6D) Various tumor cells as indicated were examined using flow cytometry making use of the noted DR5 agonist antibodies (IgG1-Fc) after treatments for the indicated times. DR5 agonist antibodies were added to the cells under the indicated conditions such as room temperature, ice, or ice combined with a crosslinking reagent. Secondary anti-IgG1-Fc and IgG1 antibody controls were included for all cell lines. (FIG. 6E) Schematic showing the murine DR5-agonist MD5-1 antibody working mechanism via anti-Fc cross-linking. (FIGS. 6F and 6G) Comparison of percent cell viability by human DR5 agonists and MD5-1 with and without an anti-Fc cross-linking secondary antibody. (FIG. 6H) Genetic construction details for generation of MD5-1 containing indicated bispecific antibodies. When shown in color, red represents MD5-1 IgG1 and blue represents Lexa or KMTR2 Or AMG655 IgG1. (FIG. 6I) Schematic of a human-murine tumor cell co-culture showing treatment with indicated antibodies followed by cell viability assays. (FIG. 6J) Cell viability assays of the indicated human-DR5 agonists, MD5-1, and various bispecific antibodies in the co-culture antibodies (n=3). Error bars in FIG. 6J represent SD (n=3).

FIGS. 7A-7M. Binding kinetics of Lexatumumab to PPCR in the Highly Variable Domain of DR5. (FIG. 7A) Determination of binding kinetics of immobilized biotinylated rDR5 having the indicated mutations against Apo2L measured using BLI. All indicated mutations interfered with Apo2L binding. (FIG. 7B) OVCAR-3 tumor cells were treated with IgG1, lexatumumab (50 nM), KMTR2 (50 nM) and lexatumumab+KMTR2 (50 nM) for 20 minutes. Cellular lysates after the indicated antibody treatments were analyzed in non-reducing denaturing gels and immunoblotting with an anti-DR5 antibody. (FIGS. 7C and 7D) Flow diagrams of for two different treatments of MDA-MB-436. In Scenario-1 (1), MDA-MB-436 tumor cells were pre-treated with KMTR2 IgG4-Fc for 20 minutes, followed by treatment with Lexa IgG1 or AMG655 IgG1 (additional 10 minutes) on ice in the presence of crosslinking solution. In Scenario-2 (2), MDA-MB-436 tumor cells were pre-treated with lexatumumab IgG4-Fc for 20 minutes, followed treatment with KMTR2 IgG1 or AMG655 IgG1 (additional 10 minutes) on ice in the presence of crosslinking solution. Immunoprecipitation was carried out using anti-IgG1-Fc specific beads followed by immunoblotting with DR5 specific antibodies. Unbound supernatant and total lysates analyzed from the same experimental conditions using immunoblotting are shown in FIG. 7D. (FIG. 7E) SDS-PAGE analysis of various indicated recombinant IgG4-Fc tagged DR5 proteins run under reducing condition. IgG1 served as a control for size. (FIG. 7F) The binding kinetics of immobilized biotinylated C103A rDR5-IgG4-Fc (RKCR-RKCR) against AMG655-IgG1, lexatumumab-IgG1, KMTR2-IgG1, and tigatuzumab IgG1 were measured. (FIG. 7G) DR5 knock-out MDA-MB-231 cells were stably transfected with lentiviruses expressing the indicated mutations in human DR5. After selection, total cellular lysates were run on a gel and immunoblotted using a commercial anti-DR5 antibody. Lane 1 has total lysates from WT MDA-MB-231 cells showing both DR5(L) larger form) and DR5 (S) shorter form). (FIGS. 7H and 71) DR5 WT(L)-, DR5 3RE(L)-, and DR5 3RA(L)-expressing MDA-MB-231 cells were incubated with KMTR2, AMG and or lexatumumab on ice in the presence of a crosslinking agent (see also FIG. 6) followed by flow cytometry analysis. KMTR2 and AMG maintained effective binding to DR5 WT(L)-, DR5 3RE(L)-, DR5 3RA(L)-expressing cells, while lexatumumab showed no binding (even after PFA crosslinking) to DR5 3RE(L)- or DR5 3RA(L)-expressing cells. (FIG. 7J) Dose dependent cell killing of DR5 knock-out MDA-MB-231 cells stably expressing either DR5 WT(L) or DR5 3RE(L) in presence of indicated DR5 agonist antibodies. When shown in color, a white circle outlined in red represents IgG Ctrl, a red circle represents L-DR5-KMTR2, a red X represents L-DR5-Tiga, a red star represents L-DR5-Lexa, a red square represents L-DR5-AMG655, a white circle outlined in blue represents IgG Ctrl, a blue circle represents 3RE-KMTR2, a blue X represents 3RE-Tiga, a blue star represents 3RE-Lexa and a blue square represents 3RE-AMG655. (FIG. 7K) Caspase-8 assay was carried out as per manufacturer's instructions using DR5 WT(L)- and DR5 3RE(L)-expressing MDA-MB-231 cells at the indicated concentrations of KMTR2 and AMG655 antibodies. Error bars are ±SEM. p values are indicated above the asterisks. (FIG. 7L) Dose dependent cell killing of DR5 knock-out MDA-MB-231 cells stably expressing either DR5 WT(L) or DR5 3RA(L) in presence of the indicated DR5 agonist antibodies. When shown in color, a white circle outlined in red represents IgG Ctrl, a red circle represents L-DR5-KMTR2, a red X represents L-DR5-Tiga, a red star represents L-DR5-Lexa, a red square represents L-DR5-AMG655, a white circle outlined in blue represents 3RA-IgG Ctrl, a blue circle represents 3RA-KMTR2, a blue X represents 3RA-Tiga, a blue star represents L-DR5-Lexa and a blue square represents 3RA-AMG655. (FIG. 2M) Fold change in IC50 values from L to RA for the indicated antibodies (n=3). Error bars represent ±SD (n=3).

FIGS. 8A-8M. 2DEI Antibody Effects on DR5. (FIG. 8A) Schematic showing potential ECD autoinhibition due to WT-DR5(L) but not DR5-3RE or DR5-3RA. (FIGS. 8B-8D) Gel analyses of WT-DR5-, DR5-3RE-, and DR5-3RA-stable MDA-MB-231 cells for DR5 clustering in non-reducing denaturing gels after the indicated antibody treatments. Total DR5 was immunoblotting in reducing condition as a loading control. (FIGS. 8E and 8F) WT-DR5(L) and DR5-3RE mutant stable MDA-MB-231 expressing an equal level of surface DR5 were treated with AMG655 antibody. Quantitated cell lysates with equal protein concentrations (from two different lines) were loaded on the same gel and analyzed for clustered DR5 using immunoblotting. Normalized quantitation of clustered DR5 signal intensity from FIG. 8G confirmed significantly high DR5 clustering in 3RE lysates. (FIGS. 8G and 8H) Immunoblotting of PARP and caspase-3 from indicated cell lysates after the indicated 2DEI and random bispecific antibody treatments (“u.c.” refers to uncleaved and “c” refers to cleaved). (FIGS. 8I and 8J) Native immunoprecipitation of DR5 with indicated DR5 agonists, 2DEI, and random bispecific antibody treatments using OVCAR-3 and MDA-MB-436 cells (“L” refers to relative confirmation and “S” refers to absolute confirmation). (FIG. 8K) Gel analyses of lysates from OVCAR-3 tumor cells treated with the indicated antibodies for 20 minutes. In additional sets, tumor cells were pre-incubated (10 minutes), either with 20-fold recombinant DR5 proteins (WT DR5, NS mut DR5, or LLF mut DR5; see also FIG. 1G) or 10-fold lexatumumab IgG1 before 2DEI antibody treatment. Cellular lysates were analyzed in non-reducing denaturing gels and immunoblotted for DR5. (FIGS. 8L and 8M) Percent survival analysis of MDA-MB-436 and HCC-1806 cells treated with the indicated 2DEI antibodies. In parallel experiments prior to the treatment with the 3 different 2DEI antibodies, corresponding monoclonal IgG1 antibodies (KMTR2, AMG655, and Tiga) were added in one experimental set or Lexa was added in a second experimental set (n=3). Error bars in represent ±SD (n=3).

FIGS. 9A-9M. 2DEI response in Cancer Animal Model. (FIG. 9A) Female NOD.Cg Prkdcscid Il2rgtm1Wjl/SzJ (NSG) mice were grafted subcutaneously with orthotopic tumors of MDA-MB-468 cells. Upon tumor generation, randomly selected animals were injected i.p. with the indicated antibodies (100 μg each) every third day, and tumor volumes were quantified at indicated days by caliper measurements. p values are noted for certain comparisons in the upper right. When shown in color, a white circle outlined in black represents IgG1, a white circle outlined in blue represents KMTR2, a white circle outlined in red represents Lexa, a black start represents AMG-Tiga and a black asterisks represents AMG-Lexa. (FIG. 9B) NSG animals were grafted to generate primary subcutaneous tumors. When tumors reached ˜700 mm³ (˜4 weeks), surgeries were performed to remove the tumors. After a 2-week recovery post-surgery, animals were injected i.p. with IgG1, KMTR2, or 2DEI (100 μg for each) every third day, and tumor-bearing animals were live imaged. Representative images at day 70 are shown after the indicated antibody treatments. Compare to FIG. 4C-4E. (FIG. 9C) Necropsies of representative animals described In FIG. 9B were recovered at day 75 and analyzed by fluorescent imaging for detailed organ specific tumor load. Quantitation of accumulated luciferase signal (radiant efficiency) from the indicated organs after KMTR2 and 2DEI antibody injections at day 75. (FIG. 9D) MDA-MB-231-2B cells (a TNBC brain metastatic derivative of human mammary cells) were injected intracardially to generate highly aggressive metastatic tumors that spread to the lungs and peritoneal cavities within 4-5 weeks. Randomly metastatic tumor-bearing animals were treated with the indicated antibodies (100 μg, 2 times a week) and were live imaged at the indicated days. Images of representative animals are shown. Compare to FIG. 4F for survival analysis. (FIG. 9E) Necropsies of the animals described in FIG. 9D from each treatment group were recovered at day 46 and analyzed by fluorescent imaging for detailed organ specific tumor burden. (FIG. 9F) Animals before the generation of experimental metastatic MDA-MB-231-2B TNBC tumors and during various indicated treatments were weighed at various time points. No significant changes in animal weights were observed in 2DEI-treated or other antibody-treated animals as compared to IgG1-treated animals. When shown in color, blue represents KMTR2, red represents Lexa, black represents 2DEI and green represents IgG1. (FIG. 9G) A bar graph showing that when patient-derived breast TNBC UCD52, WHIM-30, and HCI-01 tumors cells were tested in spheroid cultures, the 2DEI antibody was significantly effective in killing an average of 50% of tumor spheroids within 48 hours, while KMTR2 and lexatumumab were not significantly effective as compared to IgG1. (FIG. 9H) Representative live images of NOD SCID gamma (NSG) mice breast fat pad-grafted TNBC PDX UCD52 tumors treated with either PBS, KMTR2, or 2DEI antibody. Animals were imaged after 6 doses (n=3 mice). When shown in color, gray represents IgG1, blue represents KMTR2, red represents Lexa and black represents 2DEI. (FIG. 9I) Harvested tumor weights from Animals treated as described in FIG. 9H were quantified (n=3). (FIG. 9J) Various generated recombinant IgG4-Fc human DR5 with mutations/insertions in PPCR residues (see also FIGS. 4G and 4H) are shown and their binding kinetics with lexatumumab were measured using BLI. Detailed Kd, K-ON, and K-OFF rate parameters are shown. (FIG. 9K) Schematic representation of DR4-Apo2L interface near the RKCR motif. When shown in color, cyan represents the DR4 backbone, green represents Apo2L, red represents positively charged residues, blue represents negatively charged residues, and black represents disulfide bonds. Molecular interactions are shown with dotted pink lines. Despite having a larger interface atomic distance between key Apo2L Q205 residue and 90s loop EM residues (average 4.20 Å for DR5 and 6.68 Å for DR4; see Hymowitz et al., 1999; Hymowitz et al., 2000), the ionic interacting distance between Apo2L D203 against R101 (DR5) and R205 (DR4) is only 2.892 Å and 3.939 Å, respectively. Importantly, CRD3 engaging Apomab V_{H} (˜95%) also forms a salt bridge with DR5 K102 (of RKCR) via its D30. D30-K102 is only 2.430 Å apart. Noticeably, negatively charged D30 and D31 residues are structurally stabilized to generate a surface-exposed loop (in V_{H}) via hydrophobic side chains of Y32, W53 and F29 residues. Compare to FIGS. 41 and 4J. (FIGS. 9L and 9M) Schematic showing potential ECD autoinhibitory PPCR (RKCR; SEQ ID NO: 101) of DR5. Various recombinant IgG4-Fc human DR5 harboring double mutations were generated by replacing positively charged “RK” residues. Binding affinity of Lexa-IgG1 against various “RK” mutant DR5 were determined by ELISA. For FIG. 9M, when shown in color, a red circle represents RKCR WT, a black asterisks represents YYCR Hydrophobic, a black X represents LLCR Hydrophobic, a black star represents WWCR Hydrophobic, a black+represents AACR Hydrophobic, an upside down green triangle represents NNCR Polar, a green square represents TTCR Polar, a green circle represents SSCR Polar, a green triangle represents QQCR Polar, a blue square represents EECR Negatively charged, a blue circle represents DDC R negatively charged and a white square outlined in red represents KKCR Positively charged. In FIG. 9L, the pentapeptide sequences shown from top to correspond to SEQ ID NOs: 105 and 108-118, respectively.

### DETAILED DESCRIPTION

### I. General Considerations

Using Apo2L, a recent study described the importance of the GXXXG motif in the transmembrane domain of Death Receptor-5 (DR5) and the autoinhibitory anti-clustering function of the DR5 ectodomain (ECD; Pan et al., 2019). These findings challenged the long-standing view of DR5 activation. Since DR4 lacks the GXXXG motif, these results also pointed to subtle differences in the clustering mechanisms of DR4 and DR5 by Apo2L (Pan et al., 2019), which maintains broad specificity against two receptors sharing 60% sequence similarity. Apo2L does so by binding DR4 and DR5 via combinations of multiple low-affinity interactions across various regions of the ectodomain, which collectively contributes to receptor clustering mechanisms (Hymowitz et al., 1999; Hymowitz et al., 2000). Therefore, to identify DR5 selective negative regulatory and ectodomain auto-inhibitory residues, high-affinity DR5-selective multiple clinical antibodies (alongside Apo2L) were employed in the combination of functional receptor mutagenesis and in vitro clustering studies. The presently disclosed subject matter identifies the negative regulatory function of a critical patch of positively charged residues (PPCR) in the highly variable CRD3 domain of DR5. Additionally, by analytically applying the present findings, whether sustained antibody-mediated interference of negative regulatory PPCR residues resulted in supremely effective DR5 clustering and apoptotic clustering to tumors is also disclosed.

Receptor clustering is the first and critical step to activate apoptosis by DR5. The recent discovery of an autoinhibitory DR5 ectodomain has challenged the long-standing view of its mechanistic activation by the natural ligand, Apo2L (Pan et al., 2019). Since the autoinhibitory residues remained unknown, herein disclosed is characterization of a key patch of positively charged residues (PPCR) in the highly variable domain of DR5. PPCR electrostatically separates DR5 receptors to autoinhibit their clustering in the absence of ligand and antibody binding. Both mutational substitution and antibody mediated PPCR interference resulted in spontaneous DR5 clustering dimers and increased its apoptotic function. Notably, a dual-specific antibody, which enabled sustained tampering of PPCR function, exceptionally enhanced DR5 clustering, apoptotic activation, and distinctively improved survival of animals bearing aggressive metastatic and recurrent tumors, while clinically tested DR5 antibodies without PPCR blockade function were largely ineffective. As such, the presently disclosed subject matter provides unprecedented mechanistic insights into DR5 activation, and further identifies a therapeutic analytical design for clinical success.

### II. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and sub-combinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising,” which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition, and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

As use herein, the terms “administration of” and or “administering” a compound should be understood to mean providing a compound of the presently disclosed subject matter or a prodrug of a compound of the presently disclosed subject matter to a subject in need of treatment.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agent” is meant to include something being contacted with a cell population to elicit an effect, such as a drug, a protein, a peptide. An “additional therapeutic agent” refers to a drug or other compound used to treat an illness and can include, for example, an antibiotic or a chemotherapeutic agent.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom”, means reducing the severity of the symptom or the frequency with which such a symptom is experienced by a patient, or both.

As used herein, an “analog” of a chemical compound is a compound that, by way of example, resembles another in structure but is not necessarily an isomer (e.g., 5-fluorouracil is an analog of thymine).

As used herein, amino acids are represented by the full name thereof, by the three letter code corresponding thereto, and/or by the one-letter code corresponding thereto, as summarized in the following Table 1:

TABLE 1
Amino Acid Codes and Functionally Equivalent Codons

| | 3- | 1- | |
|---|---|---|---|
| | Letter | Letter | |
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is me\ant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids have the following general structure:

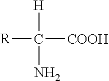

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody”, as used herein, refers to an immunoglobulin molecule which is able to specifically or selectively bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms. The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ (scFv), complementarity determining regions (CDRs), V_{L} (light chain variable region), V_{H} (heavy chain variable region), F(ab), F(ab′)₂, and any combination of those or any other functional portion of an immunoglobulin peptide capable of binding to target antigen. In some embodiments, a biologically active fragment of an antibody is a fragment that includes a paratope. In some embodiments, a biologically active fragment of an antibody is an scFv or a F(ab) fragment.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}-C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially a Fab with part of the hinge region (see Paul, 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain”, as used herein, refers to the larger of the two types of polypeptide chains present in all antibody molecules.

An “antibody light chain”, as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bird et al., 1988; Huston et al., 1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin. Humanized antibodies have been referred to as “reshaped” antibodies. Manipulation of the complementarity-determining regions (CDR) is a way of achieving humanized antibodies. See for example, Jones et al., 1986; Riechmann et al., 1988, both of which are incorporated by reference herein. For a review article concerning humanized antibodies, see Winter & Milstein, 1991, incorporated by reference herein. See also U.S. Pat. Nos. 4,816,567; 5,482,856; 6,479,284; 6,677,436; 7,060,808; 7,906,625; 8,398,980; 8,436,150; 8,796,439; and 10,253,111; and U.S. Patent Application Publication Nos. 2003/0017534, 2018/0298087, 2018/0312588, 2018/0346564, and 2019/0151448, each of which is incorporated by reference in its entirety.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

As used herein, the term “chemically conjugated”, or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to reactions as described herein. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, or other agent used in the method of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test” cell is a cell being examined.

As used herein, the term “conservative amino acid substitution” is defined herein as an amino acid exchange within one of the five groups summarized in the following Table 2:

TABLE 2
Exemplary Conservative Amino Acid Substitutions

| Group | Characteristics | Amino Acids |
|---|---|---|
| A. | Small aliphatic, nonpolar or slightly | Ala, Ser, Thr, Pro, Gly |
| | polar residues | |
| B. | Polar, negatively charged residues | Asp, Asn, Glu, Gln |
| | and their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is cancer, which in some embodiments comprises a solid tumor.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein.

As used herein, the term “fragment”, as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, in some embodiments, at least about 100 to about 200 nucleotides, in some embodiments, at least about 200 nucleotides to about 300 nucleotides, yet in some embodiments, at least about 300 to about 350, in some embodiments, at least about 350 nucleotides to about 500 nucleotides, yet in some embodiments, at least about 500 to about 600, in some embodiments, at least about 600 nucleotides to about 620 nucleotides, yet in some embodiments, at least about 620 to about 650, and most in some embodiments, the nucleic acid fragment will be greater than about 650 nucleotides in length. In the case of a shorter sequence, fragments are shorter.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′-ATTGCC-5′ and 3′-TATGGC-5′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and XBLAST programs of Altschul et al., 1990, and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, which can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

Used interchangeably herein are the terms: 1) “isolate” and “select”; and 2) “detect” and “identify”.

The term “isolated”, when used in reference to compositions and cells, refers to a particular composition or cell of interest, or population of cells of interest, at least partially isolated from other cell types or other cellular material with which it naturally occurs in the tissue of origin. A composition or cell sample is “substantially pure” when it is at least 60%, or at least 75%, or at least 90%, and, in certain cases, at least 99% free of materials, compositions, cells other than composition or cells of interest. Purity can be measured by any appropriate method, for example, by fluorescence-activated cell sorting (FACS), or other assays which distinguish cell types. Representative isolation techniques are disclosed herein for antibodies and fragments thereof.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically or selectively binds to a target compound. A ligand (e.g., an antibody) “specifically binds to”, “is specifically immunoreactive with”, “having a selective binding activity”, “selectively binds to” or “is selectively immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically or selectively binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988, for a description of immunoassay formats and conditions that can be used to determine specific immunoreactivity.

A “receptor” is a compound that specifically or selectively binds to a ligand.

A ligand or a receptor (e.g., an antibody) “specifically binds to”, “is specifically immunoreactive with”, “having a selective binding activity”, “selectively binds to” or “is selectively immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically or selectively binds under hybridization conditions to a compound polynucleotide comprising a complementary sequence; an antibody specifically or selectively binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow & Lane 1988 for a description of immunoassay formats and conditions that can be used to determine specific or selective immunoreactivity. See also the EXAMPLES set forth herein below for additional formats and conditions that can be used to determine specific or selective immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double-stranded DNA and cDNA. Furthermore, the terms, “nucleic acid”, “DNA”, “RNA” and similar terms also include nucleic acid analogs, i.e., analogs having other than a phosphodiester backbone. For example, the so-called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human and veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

As used herein, the term “secondary antibody” refers to an antibody that binds to the constant region of another antibody (the primary antibody).

As used herein, the term “single chain variable fragment” (scFv) refers to a single chain antibody fragment comprised of a heavy and light chain linked by a peptide linker. In some cases, scFv are expressed on the surface of an engineered cell, for the purpose of selecting particular scFv that bind to an antigen of interest.

As used herein, the term “mammal” refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990; Altschul et al., 1997). The default settings provided with these programs are suitable for determining substantial similarity of nucleic acid sequences for purposes of the presently disclosed subject matter.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the method of this presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell, so as to transduce and/or transform the host cell in order to promote expression (e.g., transcription and translation) of the introduced sequence. Vectors include plasmids, phages, viruses, etc.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### III. Exemplary Embodiments

In some embodiments, the presently disclosed subject matter relates to compositions comprising (a) an effective amount of a binding agent that selectively binds an RKCR (SEQ ID NO: 101) peptide motif present in a human cysteine-rich domain 3 (CRD3) of DR5 (in some embodiments, a DR5 comprising an amino acid sequence as set forth in SEQ ID NO: 62 or SEQ ID NO: 63); and (b) an effective amount of a DR5 agonist. An exemplary DR5 of the presently disclosed subject matter is a human DR5 polypeptide, which is some embodiments comprises an amino acid sequence as set forth in Accession No. NP 003833.4 (SEQ ID NO: 62) or Accession No. NP_671716.2 (SEQ ID NO: 63) of the GENBANK® biosequence database

As used herein, a “DR5 agonist” is a composition of matter which, when administered to a subject, such as a human, enhances or extends a biological activity attributable to the level or presence of DR5 in the subject. Exemplary DR5 agonists include, but are not limited to lexatumumab, conatumumab/AMG655, drozitumab, HGSTR2J/KMTRS, LBY-135, a multivalent agent (e.g., TAS266, which is a tetrameric nanobody agonist targeting DR5; Huet et al., 2012), a ligand (e.g., a TNF-related apoptosis-inducing ligand (TRAIL), such as a recombinant human TRAIL, e.g., dulanermin (also known as AMG951). In some embodiments, a DR5 agonist selectively binds an epitope in CRD1, CRD2, and/or CRD3 in DR5. In some embodiments, the DR5 agonist selectively binds and epitope in CRD1 and/or CRD2 in DR5. In some embodiments, a DR5 agonist (which in some embodiments can be a DR5-binding antibody or fragment or derivative thereof) binds to a subsequence of the DR5 CRD3 to thereby enhance a biological activity of the DR5 polypeptide.

As used herein “DR5 (S)” and “DR5 (S)” refer to a smaller form of the DR5 ectodomain, lacking the last 22 membrane proximal residues.

As used herein “DR5(L)” and DR5(L)” refer to a larger form of the DR5 ectodomain containing all outside DR5 residues including the last 22 membrane proximal residues.

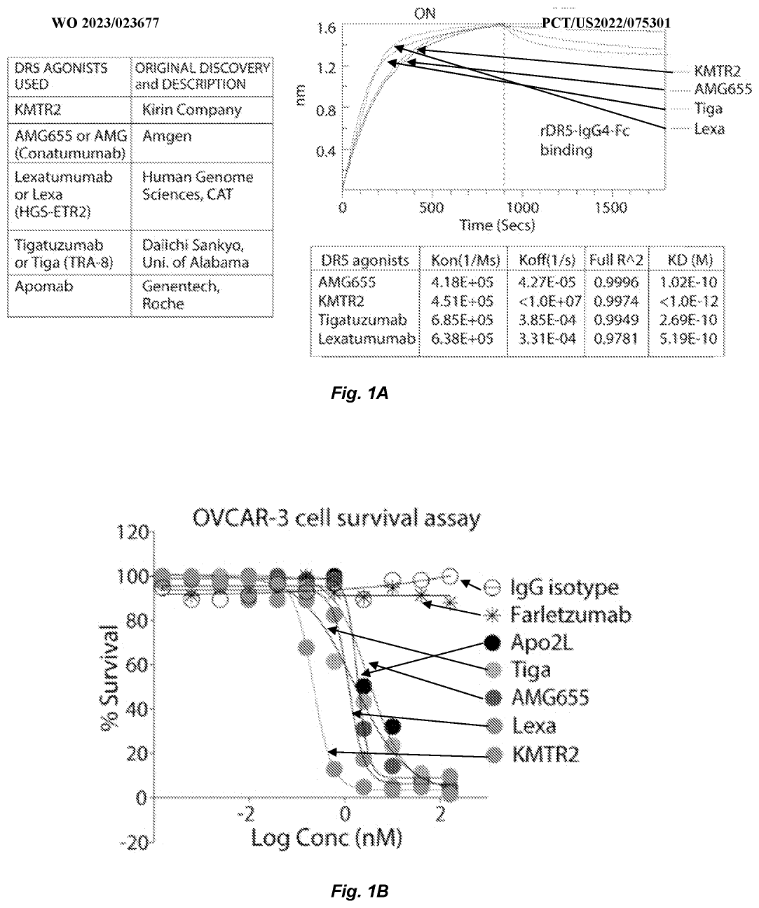

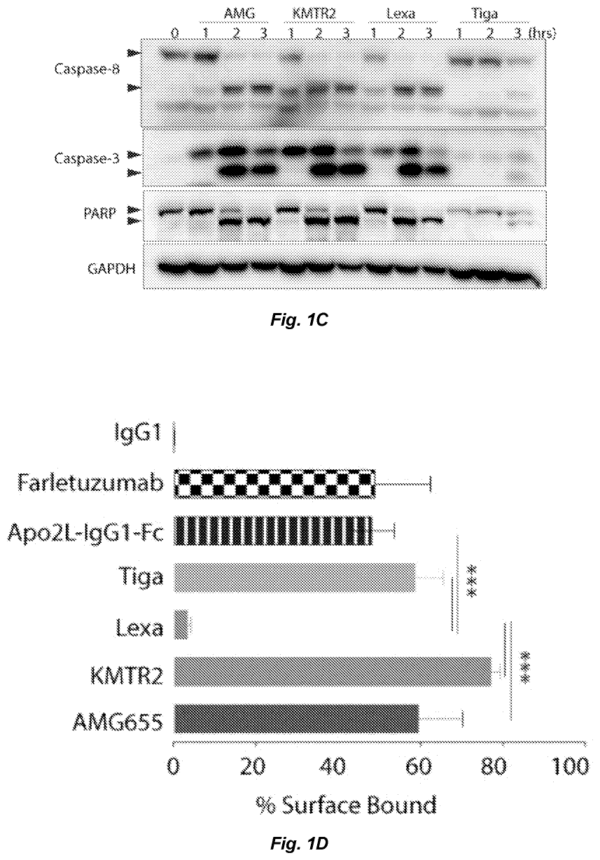

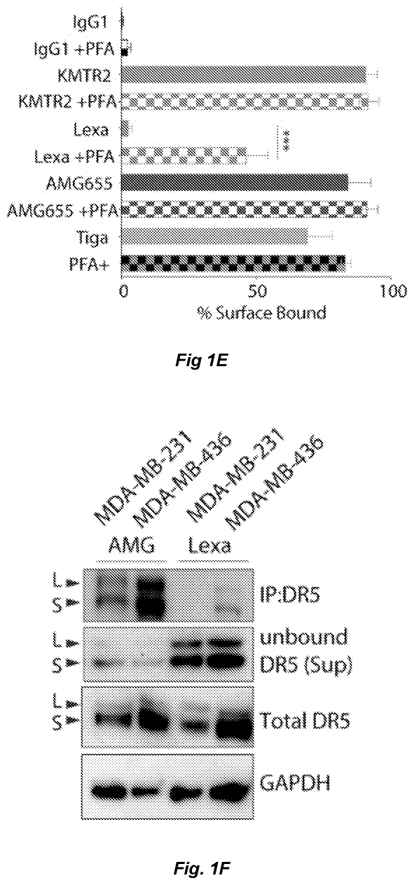

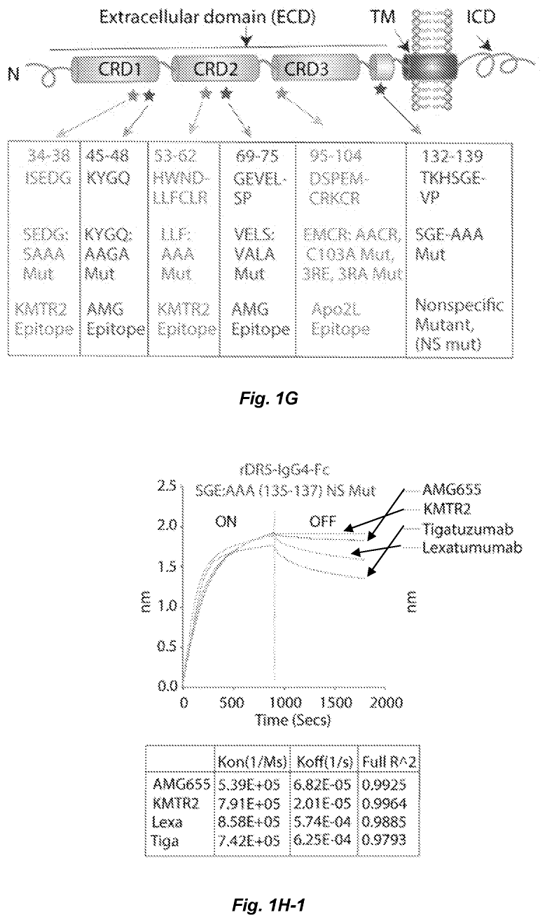

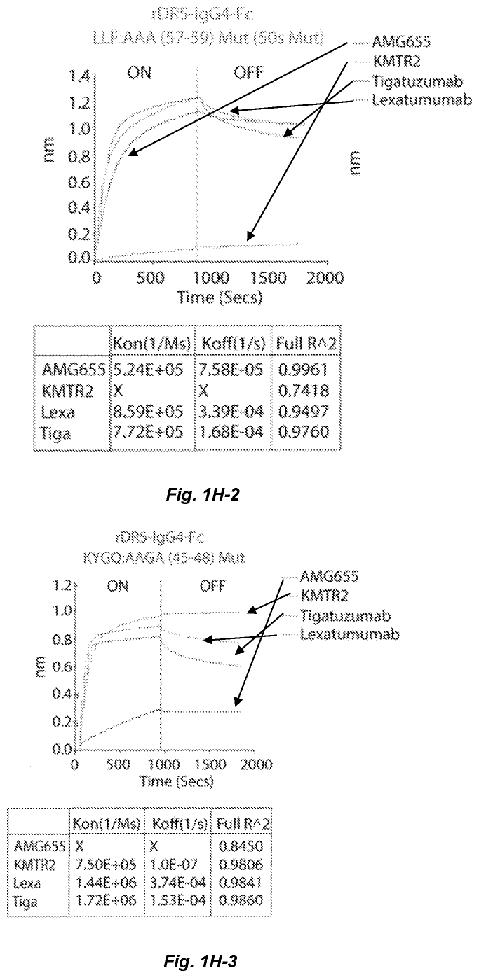

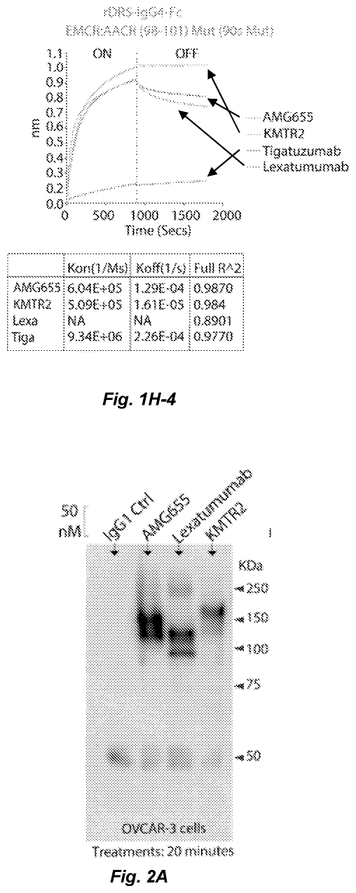

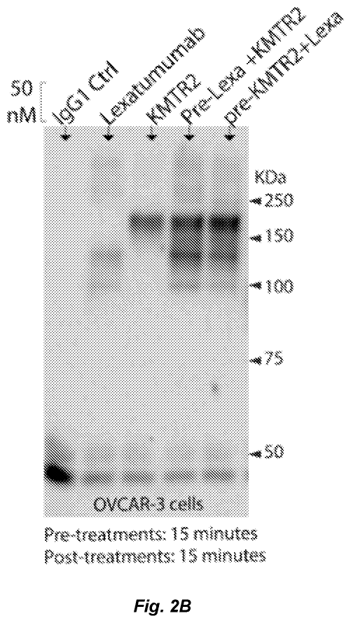

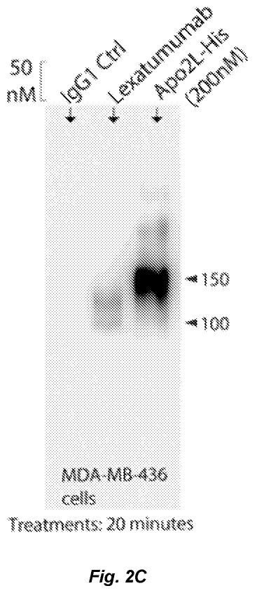

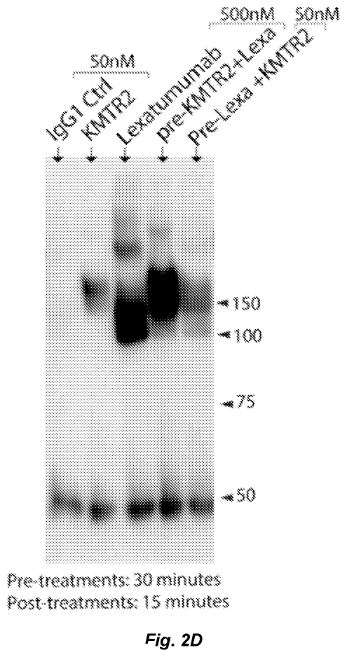

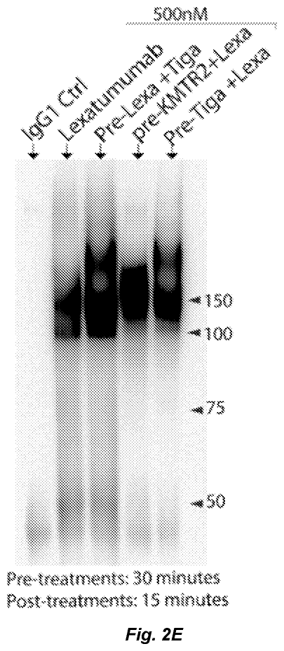

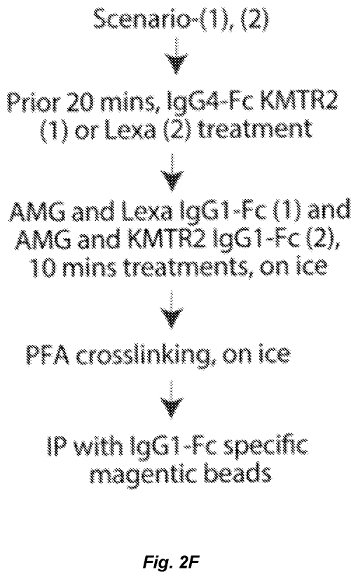

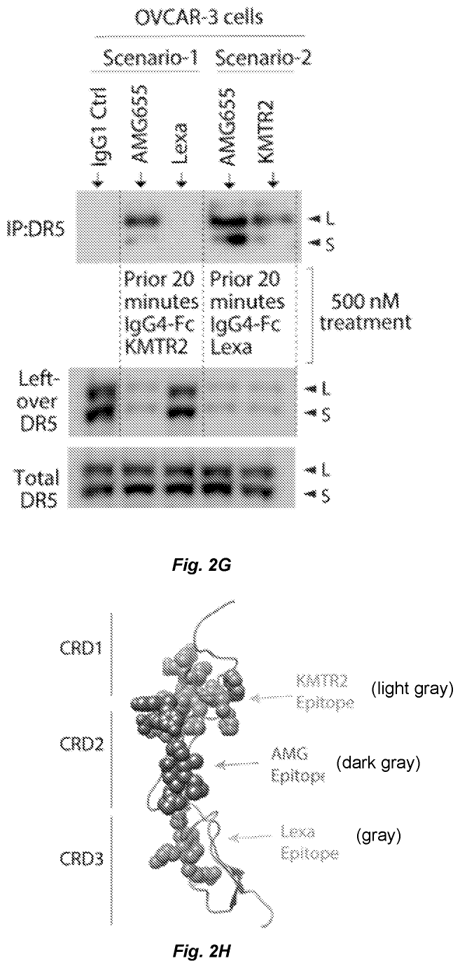

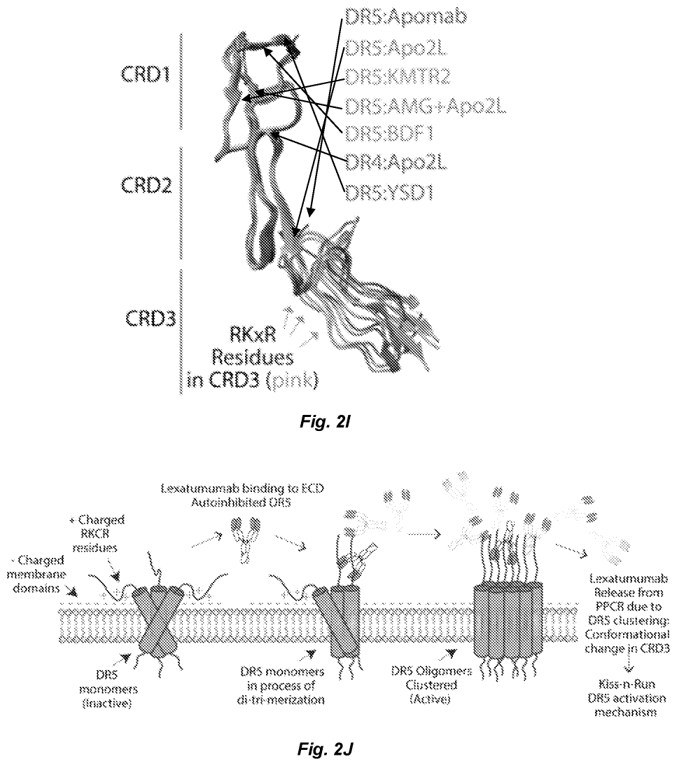

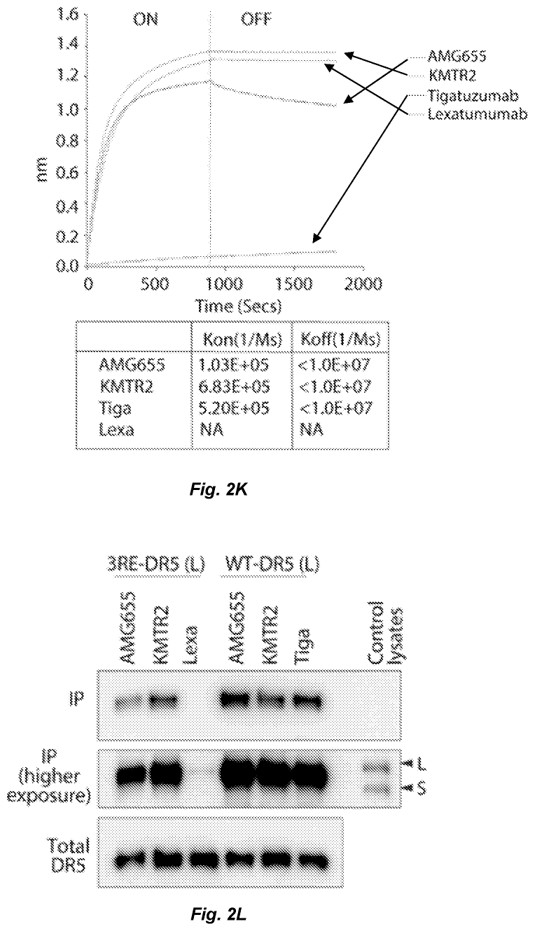

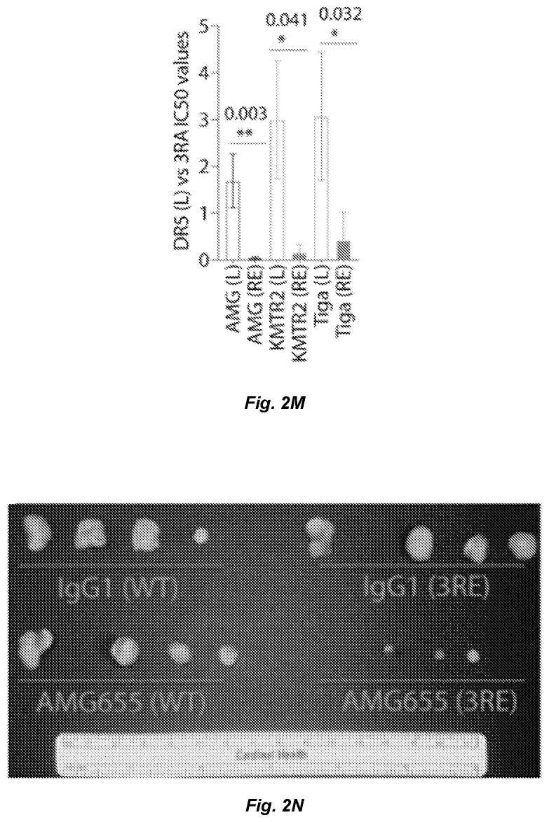

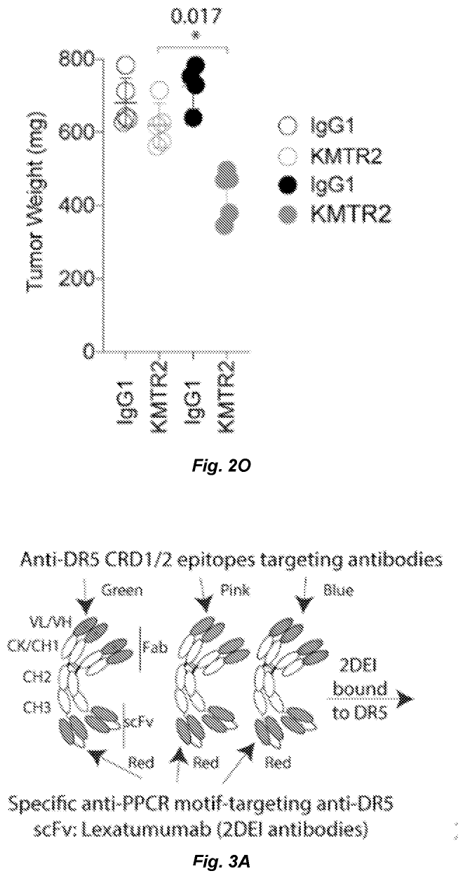

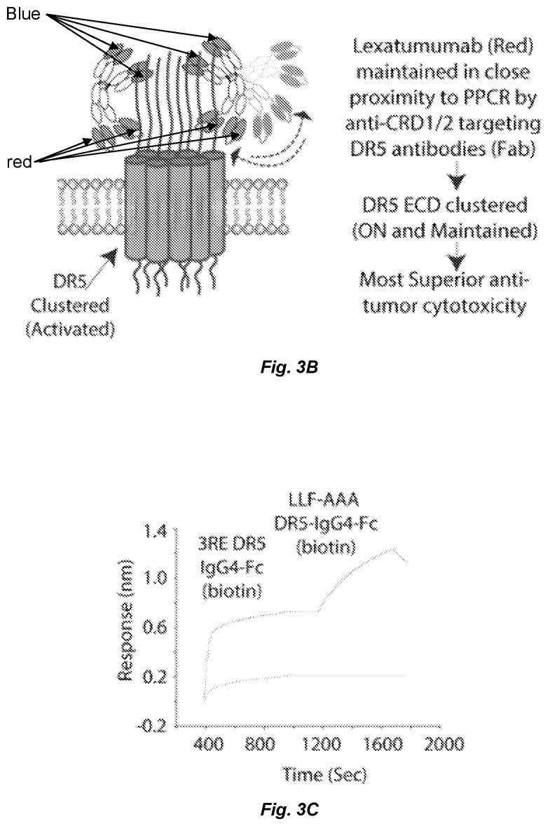

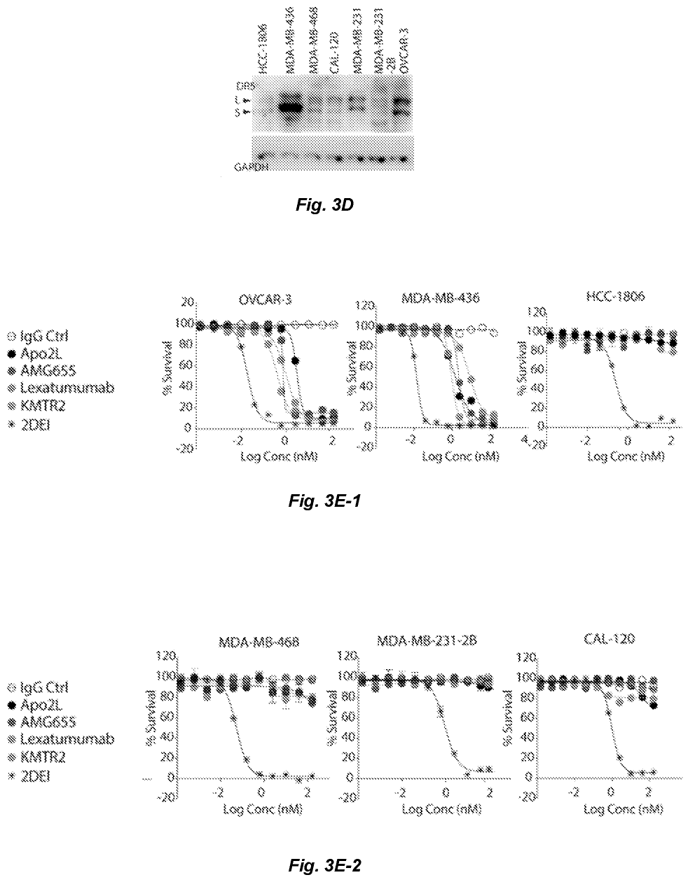

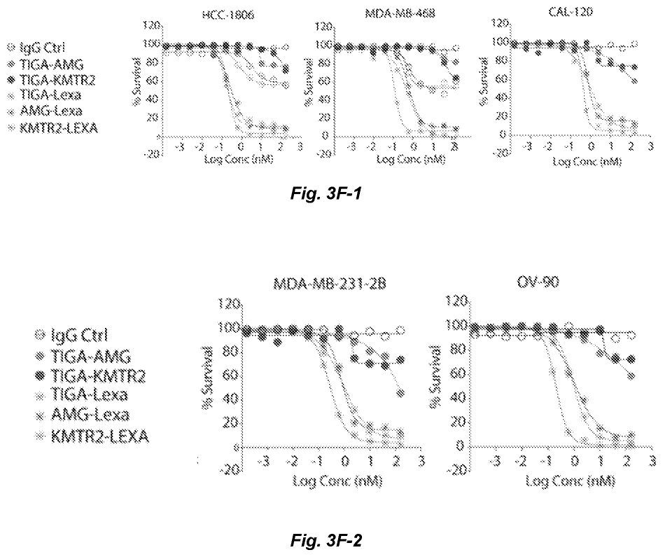

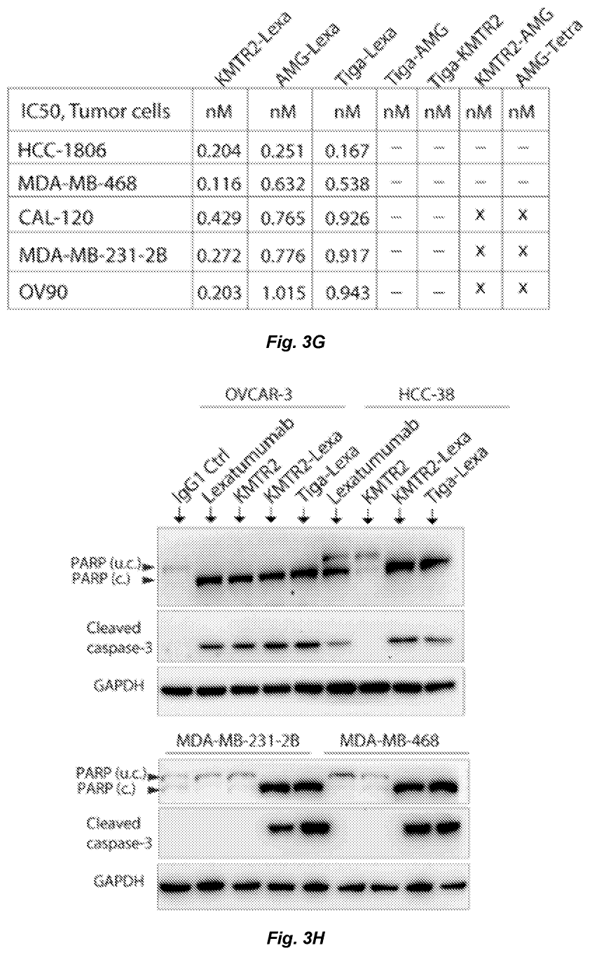

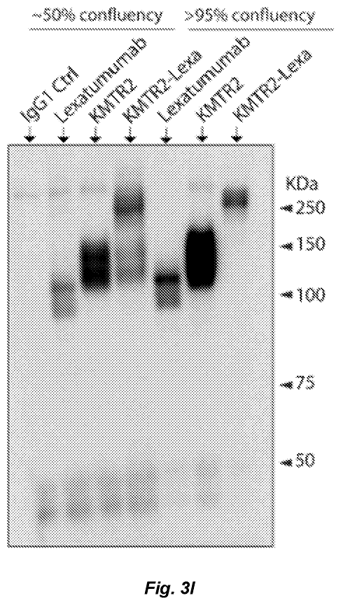

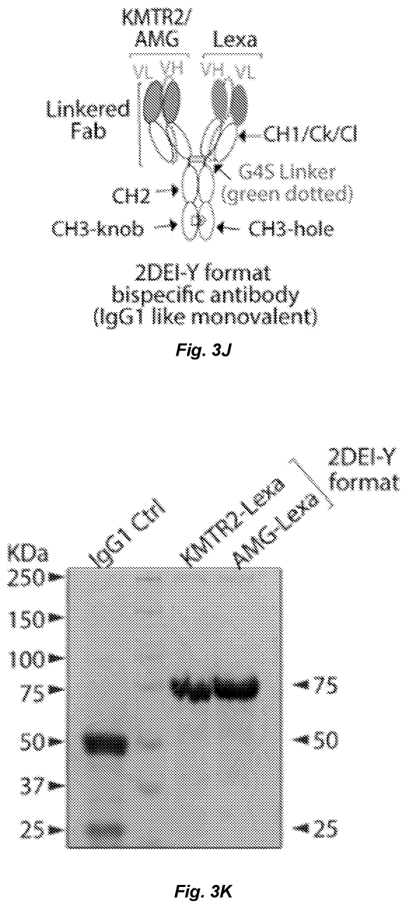

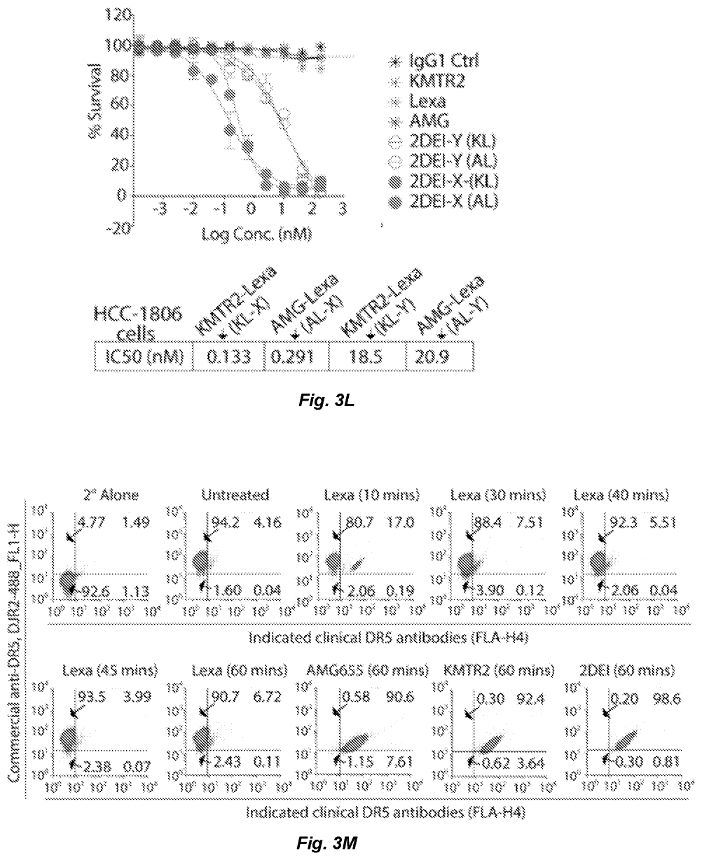

In some embodiments, the compositions of the presently disclosed subject matter comprise, consist essentially of, or consist of a 2DEI antibody. In some embodiments, an exemplary 2DEI antibody comprises a sequence as set forth in Table 3 (in some embodiments, an amino acid sequence as set forth in any one of SEQ ID NOs: 1-6), or a biologically active fragment or homolog thereof. As used herein, the phrase “2DEI” refers to a Dual DR5 Ectodomain Inhibition (2DEI) approach for configuring/preparing compositions as disclosed herein. By way of example and not limitation, and as described in the Examples presented herein below, a PPCR-engaging antibody incorporates the “kiss and run” DR5 binding and activation kinetics (see FIGS. 1 and 2J), and increases sustained interference against PPCR autoinhibition function. To this end, in some embodiments, antibodies are provided wherein a DR5 CRD3 PPCR-engaging antibody is associated with DR5 CRD1 and/or CRD2 engaging DR5 antibody to maintain the DR5 PPCR-engaging antibody sustained interference of PPCR. This antibody is referred to herein in some embodiments as a Dual DR5 Ectodomain Inhibition (2DEI) antibody (depicted generally in FIGS. 3A and 3B). In some embodiments, the compositions of the presently disclosed subject matter comprise, consist essentially of, or consist of a 2DEI antibody. In some embodiments, an exemplary 2DEI antibody comprises a sequence as set forth in Table 3, or a biologically active fragment or derivative thereof. Exemplary 2DEI antibodies of the presently disclosed subject matter include those with a heavy chain amino acid sequence as set forth in any of SEQ ID NOs: 85-94 and/or a light chain amino acid sequence as set forth in any of SEQ ID NOs: 95-100. Other 2DEI antibodies include those with amino acids sequences as set forth in any of SEQ ID NOs: 1-6, biologically active fragments thereof, including but not limited to paratope-containing fragments thereof, and homologs thereof.

As used herein, the phrase “bispecific antibody” refers to an antibody molecule that binds one antigen or epitope on one of two or more binding arms, defined by a first pair of heavy and light chains, and binds a different antigen or epitope on a second arm, defined by a second pair of heavy and light chains. Such an embodiment of a bispecific antibody has two distinct antigen binding arms, in both specificity and CDR sequences. Typically, a bispecific antibody is monovalent for each antigen it binds to. A bispecific antibody is a hybrid antibody molecule, which may have a first binding region that is defined by a first light chain variable region and a first heavy chain variable region, and a second binding region that is defined by a second light chain variable region and a second heavy chain variable region. In some embodiments, one of these binding regions may be defined by a heavy/light chain pair. As explained herein and in the context of the presently disclosed subject matter, the bispecific antibody molecule has a first binding site, defined by variable regions of a first heavy chain and a first light chain, and a second, different binding site defined by a second heavy chain and a second light chain, wherein one or both binding sites can comprise a variable region of a scFv fragment that is included in the main chain of the antibody molecule.

Methods for making a bispecific antibody molecule are known in the art, e.g. chemical conjugation of two different monoclonal antibodies or for example, also chemical conjugation of two antibody fragments, for example, of two Fab fragments. Alternatively, bispecific antibody molecules can be made recombinantly. Traditionally, the recombinant production of bispecific antibodies is based on the co-expression of two immunoglobulin H chain-L chain pairs, where the two H chains have different binding specificities. Because of the random assortment of H and L chains, a potential mixture of ten different antibody structures are produced of which only one has the desired binding specificity. An alternative approach involves fusing the variable domains with the desired binding specificities to heavy chain constant region including at least part of the hinge region, CH₂ and CH₃ regions. In one embodiment the CH₁ region containing the site necessary for light chain binding is present in at least one of the fusions. DNA encoding these fusions, and if desired the L chain are inserted into separate expression vectors and are then co-transfected into a suitable host organism. It is possible though to insert the coding sequences for two or all three chains into one expression vector.

Bispecific antibody molecules of the presently disclosed subject matter can act as a monoclonal antibody (MAb) with respect to each target. In some embodiments, the antibody is chimeric, humanized or fully human.

Thus, in some embodiments the compositions of the presently disclosed subject matter comprise, consist essentially of, or consist of one or more bispecific antibodies including, but not limited to the 2DEI antibodies disclosed herein. By way of example and not limitation, a bispecific antibody of the presently disclosed subject matter comprises a first binding art that binds to a CRD3 domain of a DR5 polypeptide, optionally wherein the binding is to a RKCR (SEQ ID NO: 101) tetrapeptide sequence present in a CRD3 domain of a DR5 polypeptide, and further optionally wherein the DR5 polypeptide is a human DR5 polypeptide. Also by way of example and not limitation, the second binding arm of the bispecific antibody binds to an epitope that is distinct from that to which the first binding arm binds. Exemplary antigens and epitopes to which the second binding arm can be designed to bind include other subsequences of the DR5 polypeptide including but not limited to subsequences of CRD1 or CRD2, an epitope other than the RKCR (SEQ ID NO: 101) tetrapeptide sequence present in the CRD3 domain, and entirely different polypeptides including but not limited to tumor-associated antigens (TAAs). Exemplary, non-limiting TAAS include Ep-CAM for breast carcinoma, BRCA1/2 for breast and ovarian carcinoma, HPV E6, E7 for cervical carcinoma, CML66, CEA, and SAP-1 for CML; TGF-βRII for colorectal carcinoma, MUCI for ductal carcinoma and RCC, Mesothelin for ductal pancreatic carcinoma, calcium-activated chloride channel 2, BING-4, Melan-A/MART-1, Gp100/pmel17, Tyrosinase, TRP-1/-2, P polypeptide, and MCIR for lung carcinoma; MART-2 and SSX-2 for melanoma, β-catenin for melanoma, prostate carcinoma, and HCC; BAGE family polypeptides, Cyclin-B1, EphA3, Her2/neu, Telomerase, Survivin, CAGE family polypeptides, GAGE family polypeptides, MAGE family polypeptides, SAGE family polypeptides, XAGE family polypeptides, NY-ESO-1/LAGE-1, PRAME, CDK4, Fibronectin, p53, Ras, Prostate-pecific antigen, TAG-72, immature laminin receptor, and 9D7.

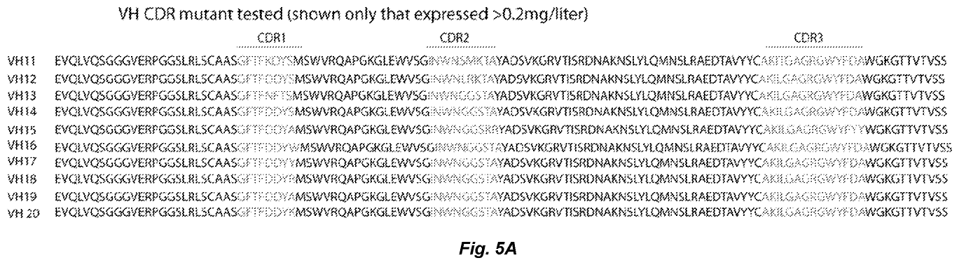

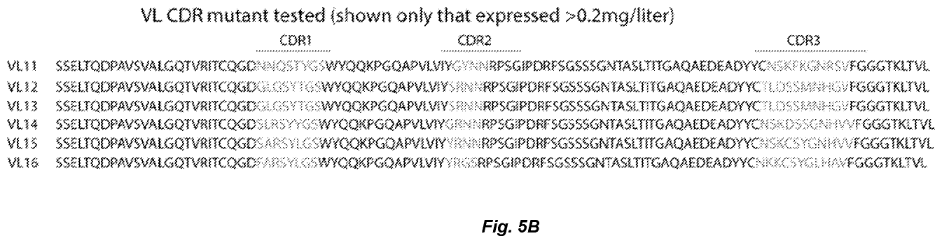

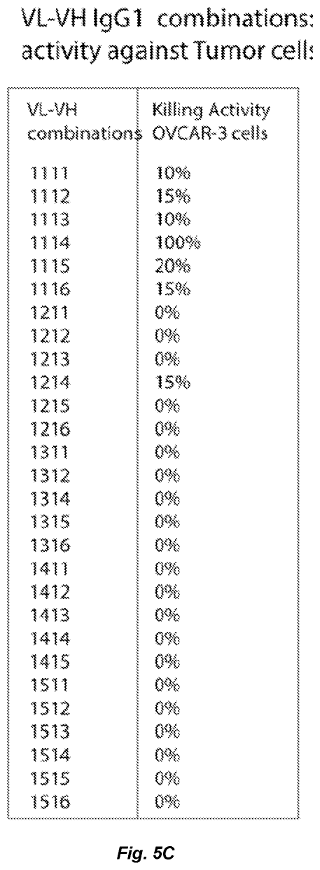

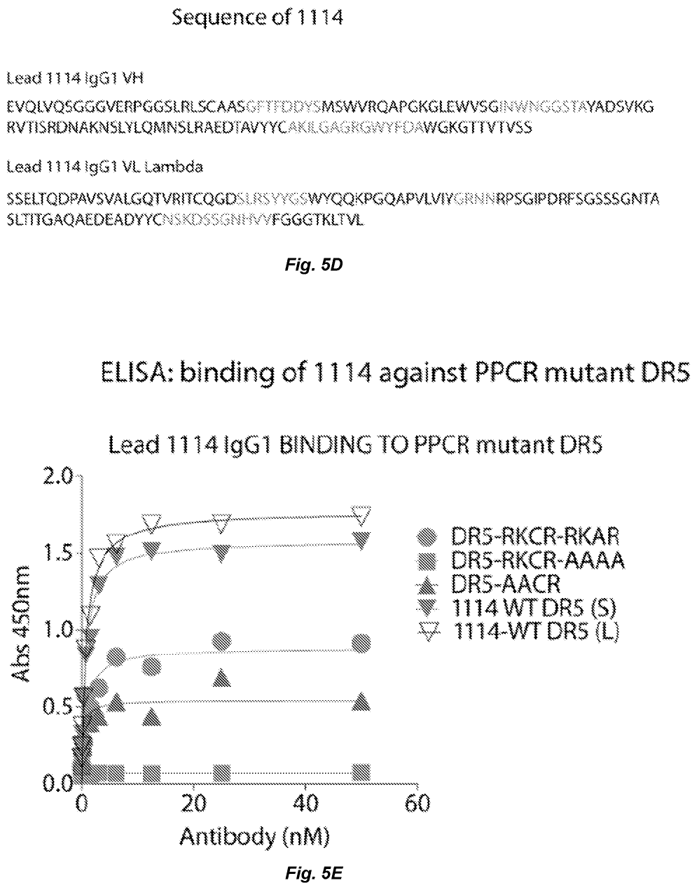

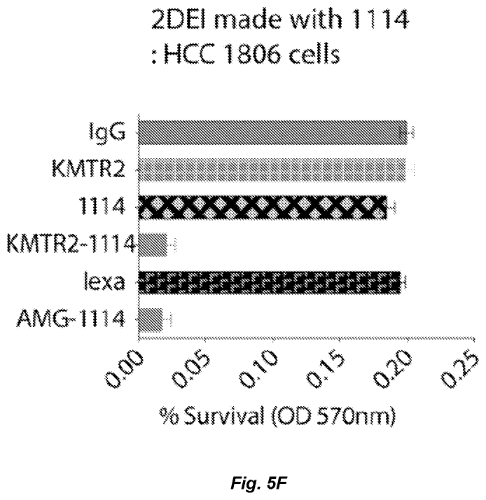

Accordingly, in some embodiments the presently disclosed subject matter relates to bispecific antibodies that bind to a death receptor 5 (DR5) polypeptide. In some embodiments, a bispecific antibody of the presently disclosed subject matter comprises a first antigen binding moiety that is specific for a peptide motif corresponding to residues 101 to 104 of a human CRD3 of DR5 (corresponds to amino acids 99-102 of SEQ ID NOs: 62 and 63, the amino acid positions 101-104 corresponding to those in certain publicly available crystal structures) and a second antigen binding moiety that is specific for an epitope of DR5 that is distinct from residues 101 to 104 of a human CRD3 of DR5 that is a DR5 agonist. In some embodiments, the first binding moeity or the second binding moeity is an antibody selected from the group comprising AMG655, KMTR2, Tigatuzumab, lexatumumab, apomab, and antibody 1114, wherein antibody 1114 comprises a heavy chain comprising an amino acid sequence as set forth in FIG. 5A and/or in any one of SEQ ID NOs: 26 and 85-94, a light chain comprising an amino acid sequence as set forth in FIG. 5B and/or in any one of SEQ ID NOs: 27 and 94-100, or is a biologically active fragment or homolog thereof. In some embodiments, the first binding moiety or the second binding moiety comprises a 2DEI antibody and/or a biologically active fragment or derivative thereof, optionally wherein the 2DEI antibody and/or the biologically active fragment or derivative thereof comprises an amino acid sequence as set forth in Table 3 (e.g., is selected from the group consisting of Tigatuzumab-1114, KMTR2-1114, AMG655-1114, KMTR2-LEXA, AMG655-LEXA, and Tigatuzumab-LEXA; SEQ ID NOs: 1-6, respectively). In some embodiments, one or both binding moieties are humanized. In some embodiments, bispecific antibodies of the presently disclosed subject matter are provided in a pharmaceutically acceptable carrier, optionally a pharmaceutically acceptable carrier that is pharmaceutically acceptable for use in a human.

In some embodiments the presently disclosed subject matter also provides antibodies that bind to a death receptor 5 (DR5) polypeptide. In some embodiments, an antibody of the presently disclosed subject matter comprises an antigen binding site that binds to the tetrapeptide motif RKCR (SEQ ID NO: 101) of a human CRD3 of DR5. In some embodiments, the antibody is antibody 1114, wherein antibody 1114 comprises a heavy chain comprising an amino acid sequence as set forth in FIG. 5A (in some embodiments, any one of SEQ ID NOs: 26 and 85-94; optionally SEQ ID NO: 26), a light chain comprising an amino acid sequence as set forth in FIG. 5B (in some embodiments, any one of SEQ ID NOs: 27 and 94-100; optionally SEQ ID NO: 27), a biologically active fragment thereof, a homolog thereof, or any combination thereof. In some embodiments, the antibodies of the presently disclosed subject matter are humanized. In some embodiments, antibodies of the presently disclosed subject matter are provided in a pharmaceutically acceptable carrier, optionally a pharmaceutically acceptable carrier that is pharmaceutically acceptable for use in a human.

In some embodiments, a bispecific antibody for treating cancer is provided in accordance with the presently disclosed subject matter.

In some embodiments of the presently disclosed subject matter, one binding function of the bispecific antibody activates cell death via engagement of Death Receptor 5 (DR5), while the other (such as but not limited to the folate receptor alpha FOLR1 highly expressed on ovarian cancer) targets the molecule to the cancer. Both binding functions of the presently disclosed bispecific antibodies bind to DR5: in some embodiments one is a high affinity antibody that binds DR5 and the other has a lower affinity that binds a key epitope for activating cell death (in some embodiments, the lexatumumab-based moiety of the bispecific antibody). A patch of positively charged residues regulates efficacy of clinical DR5 antibodies in solid tumors.

In some embodiments, the binding agent that selectively binds a peptide motif corresponding to residues 101 to 104 of a human CRD3 of DR5 (corresponds to amino acids 99-102 of SEQ ID NOs: 62 and 63) comprises an antibody. In some embodiments, the antibody is selected from the group comprising lexatumumab, apomab, and antibody 1114, wherein antibody 1114 comprises a heavy chain comprising any of the amino acid sequences disclosed in FIG. 5A (in some embodiments, one of SEQ ID NOs: 85-94) and/or a light chain comprising any of the amino acid sequences disclosed in FIG. 5B (in some embodiments, one of SEQ ID NOs: 95-100), or a biologically active fragment thereof and/or a homolog thereof.

In some embodiments, the DR5 agonist selectively binds to a CRD1 of DR5, a CDR2 of DR5, or both. In some embodiments, the DR5 agonist binds to both CRD1 and CRD2 of DR5. In some embodiments, the DR5 agonist comprises an antibody, optionally an antibody that binds to CRD1 or CRD2 of DR5. In some embodiments, the DR5 agonist comprises, consists essentially of, or consists of an antibody selected from the group consisting of AMG655, KMTR2, and tigatuzumab. In some embodiments, a binding agent that beinds to the RKCR (SEQ ID NO: 101) tetrapeptide of DR5 itself has DR5 agonist activity.

In some embodiments, the composition comprising an effective amount of a binding agent that selectively binds to a peptide motif corresponding to residues 101 to 104 of a human CRD3 of DR5 (corresponds to amino acids 99-102 of SEQ ID NOs: 62 and 63) and the composition comprising an effective amount of a DR5 agonist are provided in a single composition. In some embodiments, the single composition comprises a bispecific antibody. In some embodiments, the single composition comprises 2DEI antibody, optionally wherein the 2DEI antibody comprises a sequence as set forth in Table 3, or a biologically active fragment and/or homolog thereof. In some embodiments, the single composition comprises an antibody selected from the group comprising lexatumumab, apomab, AMG655, KMTR2, Tigatuzumab, antibody 1114 comprising a heavy chain of and/or a light chain as set forth in FIG. 5, or biologically active fragments and/or homologs thereof.

In some embodiment, the presently disclosed subject matter provides a composition comprising (a) an effective amount of a binding agent that selectively binds a peptide motif corresponding to residues 101 to 104 of a human CRD3 of DR5; and (b) an effective amount of a DR5 agonist. In some embodiments, the composition comprises a bispecific antibody. In some embodiments, the composition comprises 2DEI antibody, or a biologically active fragment and/or homolog thereof. In some embodiments, the composition further comprises a pharmaceutically acceptable carrier. In some embodiments, the DR5 agonist selectively binds at least one of a CRD1 or a CRD2 of DR5. In some embodiments, the DR5 agonist binds both CRD1 and CRD2 of DR5. In some embodiments, the DR5 agonist comprises an antibody. In some embodiments, the DR5 agonist antibody is selected from the group comprising AMG655, KMTR2, and Tigatuzumab.

In some embodiments, the presently disclosed subject matter provides a bispecific antibody that binds to death receptor 5 (DR5), wherein said bispecific antibody comprises a first antigen binding site specific for a peptide motif corresponding to residues 101 to 104 of a human CRD3 of DR5; and a second antigen binding site for DR5 that causes DR5 agonism. In some embodiments, the bispecific antibody comprises an antibody selected from the group comprising AMG655, KMTR2, and Tigatuzumab, lexatumumab, apomab, and antibody 1114 comprising a heavy chain of and/or a light chain as set forth in FIG. 5, and/or biologically active fragments and homologs thereof. In some embodiments, the bispecific antibody comprises 2DEI antibody, or a biologically active fragment and/or homolog thereof. In some embodiments, the antibody is humanized. In some embodiments, the bispecific antibody is provided in a composition comprising a pharmaceutically acceptable carrier.

In some embodiments, the presently disclosed subject matter provides an antibody that binds to death receptor 5 (DR5), wherein said antibody comprises an antigen binding site specific for a peptide motif corresponding to residues 101 to 104 of a human CRD3 of DR5; wherein the antibody comprises antibody 1114 comprising a heavy chain of and/or a light chain as set forth in FIG. 5, or biologically active fragments and/or homologs thereof. In some embodiments, the antibody is humanized. In some embodiments, the antibody is provided in a composition comprising a pharmaceutically acceptable carrier.

In some embodiments, the 1114 anti-DR5 CRD3 binding arm can be combined into bispecific antibody with any other target antigen targeting antibody too (such as including but not limited to the folate receptor (FOLR1), the epidermal growth factor receptor (EGFR), or any other tumor-specific antigen).

In some embodiments, the composition comprises a targeting moeity that targets the composition or a component thereof to a desired location (referred to as a “target” or a “targeted site”) such as, but not limited to a cell (e.g., a tumor cell and/or cancer cell), tissue, or organ. Exemplary targeting moieties include antibodies and antigen-binding fragments thereof. In some embodiments, a targeting moiety comprises an antibody or antigen-binding fragment thereof that binds to a FOLR1 gene product. As used herein, the term “FOLR1 gene product” refers to a peptide, polypeptide, or protein that is a product of the folate receptor-α (FOLR1) gene including but not limited to a human FOLR1 polypeptide. The human FOLR1 genetic locus is present on chromosome 11 and is associated with several transcription products including, but not limited to Accession Nos. NM_016724.3, NM_016725.3, NM_000802.3, and NM_0167829.3 of the GENBANK® biosequence database, which encode Accession NOs. NP_057936.1, NP_057937.1, NP_000793.1, and NP_057941.1 present therein, respectively. By way of example and not limitation, an anti-FOLR1 antibody or a fragment or derivative thereof can be Farletuzumab, which is a humanized anti-folate receptor-α antibody described, for example, in PCT International Patent Application Publication Nos. WO 2012/119077 and WO 2019/099374, in U.S. Pat. No. 9,599,621, in U.S. Patent Application Publication No. 2020/0283537, and in Konner et al., 2010, each of which is incorporated by reference in its entirety.

In some embodiments, the presently disclosed subject matter provides methods for treating cancers in subjects in need thereof. In some embodiments, the methods comprise, consist essentially of, or consist of administering to a subject a first composition comprising an effective amount of a binding agent that selectively binds a peptide motif corresponding to residues 101 to 104 of a human CRD3 of DR5; and a second composition comprising an effective amount of a DR5 agonist. In some embodiments, the first and the second compositions are a single composition. In some embodiments, the presently disclosed methods further comprise administering one or more additional anti-cancer and/or anti-tumor agents and/or treatments as part of a combination therapy. Exemplary additional anti-cancer and/or anti-tumor agents for use in the combination therapy are known to those of skill in the art and can be selected based on the type of cancer and/or tumor.

In some embodiments, the cancer comprises a solid tumor. Representative sequences for CRD1, CRD2, and CRD3 of DR5 include the following: in some embodiments, the sequence of CRD1 comprises, consists essentially of, or consists of the amino acid sequence QKRSSPSEGLCPPGHHISEDGRDCISCKYGQDYSTHWNDL (SEQ ID NO: 128); in some embodiments, the sequence of CRD2 comprises, consists essentially of, or consists of the amino acid sequence LFCLRCTRCDSGEVELSPCTTTRNTVCQCE EGTFREEDSP (SEQ ID NO: 129); and in some embodiments, the sequence of CRD3 comprises, consists essentially of, or consists of the amino acid sequence EMCRKCRTGCPRGMVKVGDCTPWSDIECVHK (SEQ ID NO: 130). In some embodiments, a biologically active fragment and/or homolog of CRD1, CRD2, and/or CRD3 of DR5 is encompassed by the presently disclosed subject matter.

### Peptide Modification and Preparation

One of ordinary skill in the art will appreciate that based on the sequences of the components of the antibodies disclosed herein they can be modified independently of one another with conservative amino acid changes, including, insertions, deletions, and substitutions, and that the valency could be altered as well. Amino acid changes (fragments and homologs) can be made independently in an antibody as well when they are being used in a therapy.

The presently disclosed subject matter provides other antibodies and biologically active fragments and homologs thereof as well as methods for preparing and testing new antibodies for the properties disclosed herein.

In some embodiments, the fragments are fragments of scFv. In some embodiments, the scFv fragments are mammalian. In some embodiments, the scFv fragments are humanized.

In some embodiments, the presently disclosed subject matter uses a biologically active antibody or biologically active fragment or homolog thereof. In some embodiments, the isolated polypeptide comprises a mammalian molecule at least about 30% homologous to a polypeptide having the amino acid sequence of at least one of the sequences disclosed herein. In some embodiments, the isolated polypeptide is at least about 35% homologous, more in some embodiments, about 40% homologous, more in some embodiments, about 45% homologous, in some embodiments, about 50% homologous, more in some embodiments, about 55% homologous, in some embodiments, about 60% homologous, more in some embodiments, about 65% homologous, in some embodiments, more in some embodiments, about 70% homologous, more in some embodiments, about 75% homologous, in some embodiments, about 80% homologous, more in some embodiments, about 85% homologous, more in some embodiments, about 90% homologous, in some embodiments, about 95% homologous, more in some embodiments, about 96% homologous, more in some embodiments, about 97% homologous, more in some embodiments, about 98% homologous, and most in some embodiments, about 99% homologous to at least one of the peptide sequences disclosed herein.

The presently disclosed subject matter further encompasses modification of the antibodies and fragments thereof disclosed herein, including amino acid deletions, additions, and substitutions, particularly conservative substitutions. The presently disclosed subject matter also encompasses modifications to increase in vivo half-life and decrease degradation in vivo. Substitutions, additions, and deletions can include, for example, 1, 2, 3, 4, 5, 6, 7, 8, 9, 10, 11, 12, 13, 14, 15, 16, 17, 18, 19, 20, 21, 22, 23, 24, and 25 changes as long as the activity disclosed herein remains substantially the same.

The presently disclosed subject matter includes an isolated nucleic acid comprising a nucleic acid sequence encoding an antibody of the presently disclosed subject matter, or a fragment or homolog thereof. In some embodiments, the nucleic acid sequence encodes a peptide comprising an antibody sequence of the presently disclosed subject matter, or a biologically active fragment of homolog thereof.

In some embodiments, a homolog of a peptide (antibody or fragment) of the presently disclosed subject matter is one with one or more amino acid substitutions, deletions, or additions, and with the sequence identities described herein. In some embodiments, the substitution, deletion, or addition is conservative.

The presently disclosed subject matter encompasses the use of purified isolated, recombinant, and synthetic peptides.

It will be appreciated, of course, that the proteins or peptides of the presently disclosed subject matter may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (−NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for analogs of proteins, e.g., analogs of antibodies. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the presently disclosed subject matter are not limited to products of any of the specific exemplary processes listed herein.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Substantially pure protein obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability, and/or other biological, physical, and chemical properties.

### Amino Acid Substitutions

In certain embodiments, the disclosed methods and compositions may involve preparing peptides with one or more substituted amino acid residues.

In various embodiments, the structural, physical, and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic, and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′, -3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C1-C10 branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 in some embodiments can be employed, within +/−1 in some embodiments can be employed, and within +/−0.5 in some embodiments can be employed.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In some embodiments, replacement of amino acids with others of similar hydrophilicity is employed.

Other considerations include the size of the amino acid side chain. For example, in some embodiments an amino acid with a compact side chain, such as glycine or serine, would not be replaced with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; and Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine, and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser(S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, Mclachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### Antibody Formats And Preparation Thereof

Antibodies directed against proteins, polypeptides, or peptide fragments thereof of the presently disclosed subject matter may be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In some embodiments, one or more antibodies or fragments thereof are used. In some embodiments, one or both antibodies are single chain, monoclonal, bi-specific, synthetic, polyclonal, chimeric, human, or humanized, or active fragments or homologs thereof. In some embodiments, the antibody binding fragment is scFV, F(ab′)₂, F(ab)₂, Fab′, or Fab.

For the preparation of monoclonal antibodies, any technique which provides for the production of antibody molecules by continuous cell lines in culture may be utilized. For example, the hybridoma technique originally developed by Kohler & Milstein, the trioma technique, the human B-cell hybridoma technique (Kozbor & Roder, 1983), and the EBV-hybridoma technique (Cole et al., 1985) may be employed to produce human monoclonal antibodies. In some embodiments, monoclonal antibodies are produced in germ-free animals.

In accordance with the presently disclosed subject matter, human antibodies may be used and obtained by utilizing human hybridomas (Cote et al., 1985) or by transforming human B cells with EBV virus in vitro (Cole et al., 1985). Furthermore, techniques developed for the production of “chimeric antibodies” (Morrison et al., 1984; Neuberger et al., 1984; Takeda et al., 1985) by splicing the genes from a mouse antibody molecule specific for epitopes of SLLP polypeptides together with genes from a human antibody molecule of appropriate biological activity can be employed; such antibodies are within the scope of the presently disclosed subject matter. Once specific monoclonal antibodies have been developed, the preparation of mutants and variants thereof by conventional techniques is also available.

Various techniques have been developed for the production of antibody fragments of humanized antibodies. Traditionally, these fragments were derived via proteolytic digestion of full-length antibodies (see e.g., Morimoto & Inouye, 1992; Brennan et al., 1985). However, these fragments can now be produced directly by recombinant host cells. Alternatively, Fab′-SH fragments can be directly recovered from E. coli and chemically coupled to form F(ab′)₂ fragments (Carter et al., 1992a). According to another approach, F(ab′)₂ fragments can be isolated directly from recombinant host cell culture. Other techniques for the production of antibody fragments will be apparent to the skilled practitioner. In other embodiments, the antibody of choice is a single-chain Fv fragment (scFv). See PCT International Patent Application Publication No. WO 1993/16185; U.S. Pat. Nos. 5,571,894; 5,587,458. The antibody fragment may also be a “linear antibody”, e.g., as described in U.S. Pat. No. 5,641,870, for example. Such linear antibody fragments may be monospecific or bispecific.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see e.g., U.S. Pat. Nos. 4,975,369; 5,075,431; 5,081,235; 5,169,939; 5,202,238; 5,204,244; 5,231,026; 5,292,867; 5,354,847; 5,472,693; 5,482,856; 5,491,088; 5,500,362; and 5,502,167). Detailed methods for preparation of chimeric (humanized) antibodies can be found in U.S. Pat. No. 5,482,856. A “humanized” antibody is a human/non-human chimeric antibody that contains a minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or non-human primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, a humanized antibody will comprise substantially all of at least one, and typically two, variable domains, in which all or substantially all of the hypervariable loops correspond to those of a non-human immunoglobulin and all or substantially all of the FR residues are those of a human immunoglobulin sequence. The humanized antibody can optionally also comprise at least a portion of an immunoglobulin constant region (Fc), typically that of a human immunoglobulin. For further details, see e.g., Jones et al., 1986; Riechmann et al., 1988; Presta, 1992, PCT International Patent Application Publication No. WO 92/02190, U.S. Patent Application Publication No. 2006/0073137, and U.S. Pat. Nos. 5,225,539; 5,530,101; 5,585,089; 5,693,761; 5,693,762; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,548,640; 6,632,927; 6,639,055; and 6,750,325.

In some embodiments, the presently disclosed subject matter provides for fully human antibodies. Human antibodies consist entirely of characteristically human polypeptide sequences. The human antibodies of this presently disclosed subject matter can be produced in using a wide variety of methods (see e.g., U.S. Pat. No. 5,001,065, for review).

Typically, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al, 1986; Riechmann et al., 1988; Verhoeyen & Riechmann, 1988), by substituting hypervariable region sequences for the corresponding sequences of a human “acceptor” antibody. Accordingly, such “humanized” antibodies are chimeric antibodies (see e.g., U.S. Pat. Nos. 4,816,567 and 5,482,856) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

Another method for making humanized antibodies is described in U.S. Patent Application Publication No. 2003/0017534, wherein humanized antibodies and antibody preparations are produced from transgenic non-human animals. The non-human animals are genetically engineered to contain one or more humanized immunoglobulin loci that are capable of undergoing gene rearrangement and gene conversion in the transgenic non-human animals to produce diversified humanized immunoglobulins.

In some embodiments, the choice of human variable domains, both light and heavy, to be used in making the humanized antibodies is very important to reduce antigenicity. According to the so-called “best-fit” method, the sequence of the variable domain of a rodent antibody is screened against a library of known human variable-domain sequences or a library of human germline sequences. The human sequence that is closest to that of the rodent can then be accepted as the human framework region for the humanized antibody (Sims et al., 1993; Chothia & Lesk, 1987). Another method uses a particular framework region derived from the consensus sequence of all human antibodies of a particular subgroup of light or heavy chains. The same framework may be used for several different humanized antibodies (Carter et al., 1992b; Presta et al., 1993). Other methods designed to reduce the immunogenicity of the antibody molecule in a human patient include veneered antibodies (see e.g., U.S. Pat. No. 6,797,492 and U.S. Patent Application Publication Nos. 2002/0034765 and 2004/0253645) and antibodies that have been modified by T-cell epitope analysis and removal (see e.g., U.S. Patent Application Publication No. 2003/0153043 and U.S. Pat. No. 5,712,120).

It is important that when antibodies are humanized, they retain high affinity for the antigen and other favorable biological properties. To achieve this goal, according to one method, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

The antibody moieties of this presently disclosed subject matter can be single chain antibodies.

Antibodies directed against proteins, polypeptides, or peptide fragments thereof of the presently disclosed subject matter may be generated using methods that are well known in the art. For instance, U.S. Pat. No. 5,436,157, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Patent Application No. 2003/0022244.

Fragments within the scope of the term “antibody” include those produced by digestion with various proteases, those produced by chemical cleavage and/or chemical dissociation and those produced recombinantly, so long as the fragment remains capable of specific binding to a target molecule. Among such fragments are Fab, Fab′, Fv, F(ab′)₂, and single chain Fv (scFv) fragments.

In some embodiments, the specific binding molecule is a single-chain variable fragment (scFv). The specific binding molecule or scFv may be linked to other specific binding molecules (for example other scFvs, Fab antibody fragments, chimeric IgG antibodies (e.g., with human frameworks)) or linked to other scFvs of the presently disclosed subject matter so as to form a multimer which is a multi-specific binding protein, for example a dimer, a trimer, or a tetramer. Bi-specific scFvs are sometimes referred to as diabodies, tri-specific such as triabodies and tetra-specific such as tetrabodies when each scFv in the dimer, trimer, or tetramer has a different specificity. Diabodies, triabodies and tetrabodies can also be monospecific, when each scFv in the dimer, trimer, or tetramer has the same specificity.

In some embodiments, techniques described for the production of single-chain antibodies (U.S. Pat. No. 4,946,778, incorporated by reference herein in its entirety) are adapted to produce protein-specific single-chain antibodies. In some embodiments, the techniques described for the construction of Fab expression libraries (Huse et al., 1989) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which bind the antigen therefrom at any epitopes present therein.

Monoclonal antibodies directed against full length or peptide fragments of a protein or peptide may be prepared using any well-known monoclonal antibody preparation procedures, such as those described, for example, in Harlow & Lane, 1988; Tuszynski et al., 1988). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Exemplary complementarity-determining region (CDR) residues or sequences and/or sites for amino acid substitutions in framework region (FR) of such humanized antibodies having improved properties such as, e.g., lower immunogenicity, improved antigen-binding or other functional properties, and/or improved physicochemical properties such as, e.g., better stability, are provided.

The presently disclosed subject matter encompasses more than the specific fragments and humanized fragments disclosed herein. In some embodiments, the antibody is selected from the group consisting of a single chain antibody, a monoclonal antibody, a bi-specific antibody, a chimeric antibody, a synthetic antibody, a polyclonal antibody, or a humanized antibody, or active fragments or homologs thereof.

A nucleic acid encoding the monoclonal antibody obtained using the procedures described herein may be cloned and sequenced using technology that is available in the art, and is described, for example, in Wright et al., 1992 and the references cited therein. Further, the antibody of the presently disclosed subject matter may be “humanized” using the technology described in Wright et al., 1992 and in the references cited therein, and in Nasoff et al., 1997.

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Green & Sambrook, 2012.

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton & Barbas, 1994). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

In accordance with the presently disclosed subject matter, human antibodies may be used and obtained by utilizing human hybridomas (Cote et al., 1983) or by transforming human B cells with EBV virus in vitro (Cole et al., 1985). Furthermore, techniques developed for the production of “chimeric antibodies” (Morrison et al., 1984; Neuberger et al., 1984; Takeda et al., 1985).

The procedures just presented describe the generation of phage which encode the Fab portion of an antibody molecule. However, the presently disclosed subject matter should not be construed to be limited solely to the generation of phage encoding Fab antibodies. Rather, phage which encode single chain antibodies (scFv/phage antibody libraries) are also included in the presently disclosed subject matter. Fab molecules comprise the entire Ig light chain, that is, they comprise both the variable and constant region of the light chain but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions may be synthesized such that they include nearly all possible specificities (Barbas 3rd, 1995; de Kruif et al., 1995).

In the production of antibodies, screening for the desired antibody can be accomplished by techniques known in the art, e.g., ELISA (enzyme-linked immunosorbent assay). Antibodies generated in accordance with the presently disclosed subject matter may include, but are not limited to, polyclonal, monoclonal, chimeric (i.e., “humanized”), and single chain (recombinant) antibodies, Fab fragments, and fragments produced by a Fab expression library.

Substantially pure peptide obtained as described herein may be purified by following known procedures for protein purification, wherein an immunological, enzymatic, or other assay is used to monitor purification at each stage in the procedure. Protein purification methods are well known in the art, and are described, for example in Deutscher et al., 1990.

It is common in the field of recombinant humanized antibodies to graft murine CDR sequences onto a well-established human immunoglobulin framework previously used in human therapies such as the framework regions of Herceptin [Trastuzumab].

In some embodiments, when used in vivo for therapy, the antibodies of the subject presently disclosed subject matter are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

In some embodiments, for parenteral administration, the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

### Pharmaceutical Compositions and Administration

The presently disclosed subject matter is also directed to methods of administering the compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active peptide, one or more acceptable carriers, and optionally other peptides or therapeutic agents.

For in vivo applications, the peptides of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The peptides of the presently disclosed subject matter, pharmaceutically acceptable salts thereof, or pharmaceutical compositions comprising these compounds may be administered so that the compounds may have a physiological effect. Administration may occur enterally or parenterally; for example, orally, rectally, intracisternally, intravaginally, intraperitoneally, locally (e.g., with powders, ointments or drops), or as a buccal or nasal spray or aerosol. In some embodiments, parenteral administration is employed. Exemplary parenteral administration methods include intravascular administration (e.g., intravenous bolus injection, intravenous infusion, intra-arterial bolus injection, intra-arterial infusion and catheter instillation into the vasculature), peri- and intra-target tissue injection (e.g., peri-tumoral and intra-tumoral injection), subcutaneous injection or deposition including subcutaneous infusion (such as by osmotic pumps), intramuscular injection, and direct application to the target area, for example by a catheter or other placement device.

Where the administration of the peptide is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, 1985, which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also provides in some embodiments methods for activating DR5 biological activities in cells, tissues, and/or organs, optionally cells, tissues, and/or organ present in subjects. In some embodiments, the methods comprise, consist essentially of, or consisting of administering to a subject in need thereof (a) a first composition comprising an effective amount of a binding agent that selectively binds to an RKCR (SEQ ID NO: 101) tetrapeptide motif of a human CRD3 of DR5; and (b) a second composition comprising, consisting essentially of, or consisting of an effective amount of a DR5 agonist. In some embodiments, the DR5 biological activity to be activated is present in a cell of a tumor or a cancer, optionally a cell of a solid tumor. In some embodiments, the binding agent comprises an antibody, optionally, an antibody of the presently disclosed subject matter such as but not limited to an antibody that is selected from the group comprising lexatumumab and antibody 1114, wherein antibody 1114 comprises a heavy chain comprising an amino acid sequence as set forth in FIG. 5A (in some embodiments, any one of SEQ ID NOs: 85-94), a light chain comprising an amino acid sequence as set forth in FIG. 5B (in some embodiments, any one of SEQ ID NOs: 95-100), a biologically active fragment thereof, a homolog thereof, or any combination thereof. In some embodiments, the DR5 agonist selectively binds at least one of a CRD1 and a CRD2 of DR5. In some embodiments, the DR5 agonist comprises an antibody, optionally an anti-DR5 antibody or a DR5-binding fragment or derivative thereof, further optionally an antibody that binds to at least one of a CRD1 and a CRD2 of DR5. In some embodiments, a DR5 agonist is a peptide that binds to CRD1 and/or CRD2 to provide an anchor to an anti-CRD3 antibody for DR5 agonist activity. In some embodiments, the anti-CRD3 antibody itself has DR5 agonist activity.

In some embodiments of the presently disclosed methods, the first composition and the second composition are provided in a single composition. In some embodiments, the single composition comprises a bispecific antibody as disclosed herein. In some embodiments, the bispecific antibody comprises a first arm that binds to DR5 CRD3, optionally to the tetrapeptide RKCR (SEQ ID NO: 101) located therein. In some embodiments, the bispecific antibody comprises a second arm that binds to DR5, in some embodiments to CRD1, CRD2, or CRD3 of DR5, and in some embodiments outside of CRD1, CRD2, and CRD3 of DR5. In some embodiments, the second arm that binds to a tumor-associated antigen (TAA). In some embodiments, the TAA is selected from the group consisting of EGFR and FOLR1, although it is recognized that the second binding arm can also be designed to bind to other TAAs.

In some embodiments, the single composition comprises a 2DEI antibody as disclosed herein, which in some embodiments can be a 2DEI antibody that comprises a sequence as set forth in Table 3, in some embodiments an amino acid sequence as set forth in any one of SEQ ID NOs: 1-6, or a biologically active fragment and/or homolog thereof. By way of example and not limitation, in some embodiments the single composition can comprise an antibody selected from the group comprising lexatumumab and antibody 1114 (e.g., an antibody that comprises a heavy chain comprising an amino acid sequence as set forth in FIG. 5A (in some embodiments, any one of SEQ ID NOs: 85-94), a light chain comprising an amino acid sequence as set forth in FIG. 5B (in some embodiments, any one of SEQ ID NOs: 95-100), a biologically active fragment thereof, a homolog thereof, or any combination thereof.

The presently disclosed subject matter also includes a kit comprising the composition of the presently disclosed subject matter and an instructional material which describes administering the composition to a subject. In some embodiments, this kit comprises a (in some embodiments sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of a composition of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains a composition of the presently disclosed subject matter or be shipped together with a container which contains the composition. Alternatively, the instructional material may be shipped separately from the container with the intention that the instructional material and the compound be used cooperatively by the recipient.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Example 1

### Lexatumumab Activates Efficient Cell-Death Despite its Unexpectedly Low Surface Binding

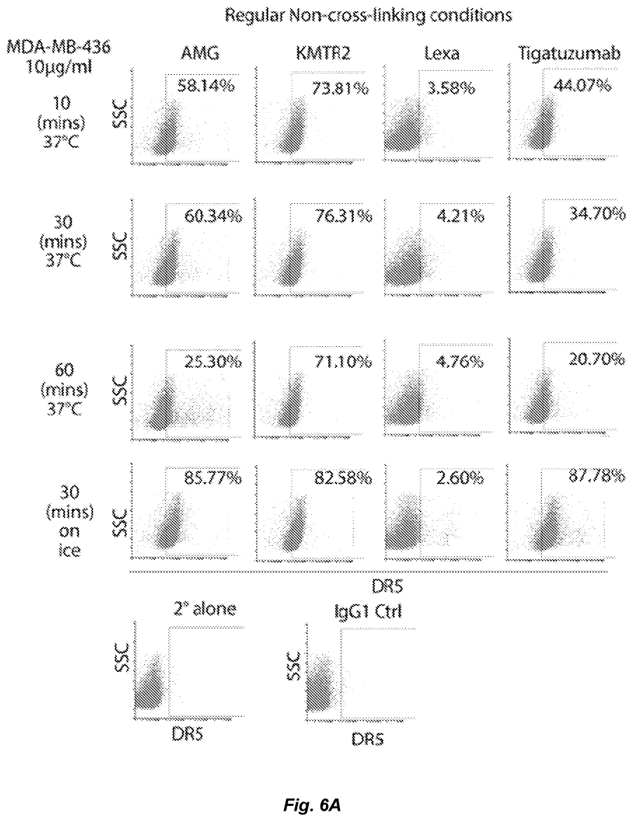

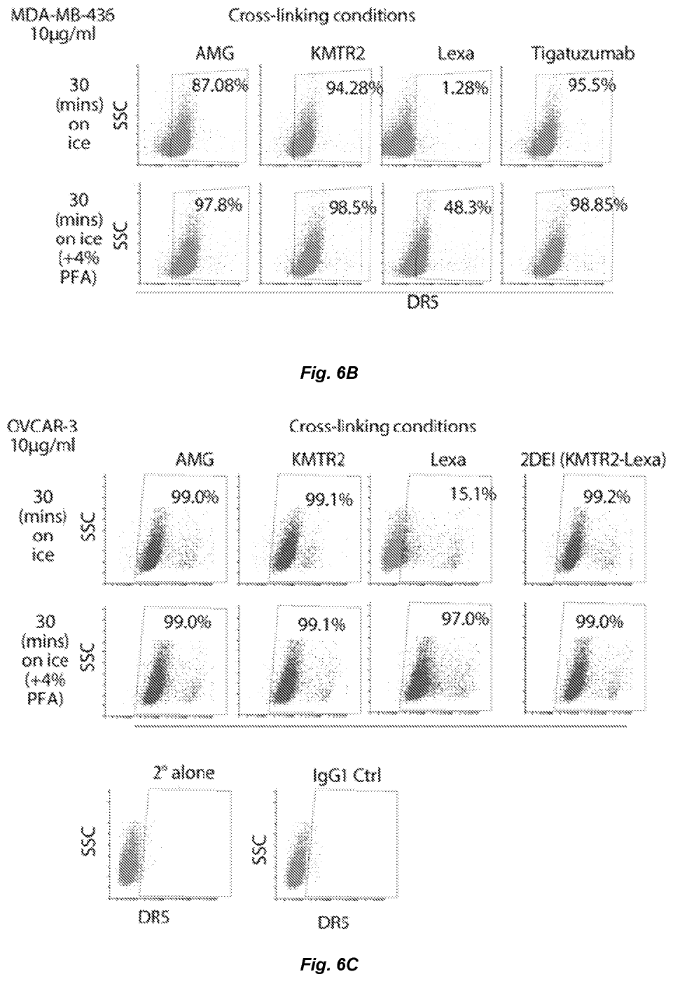

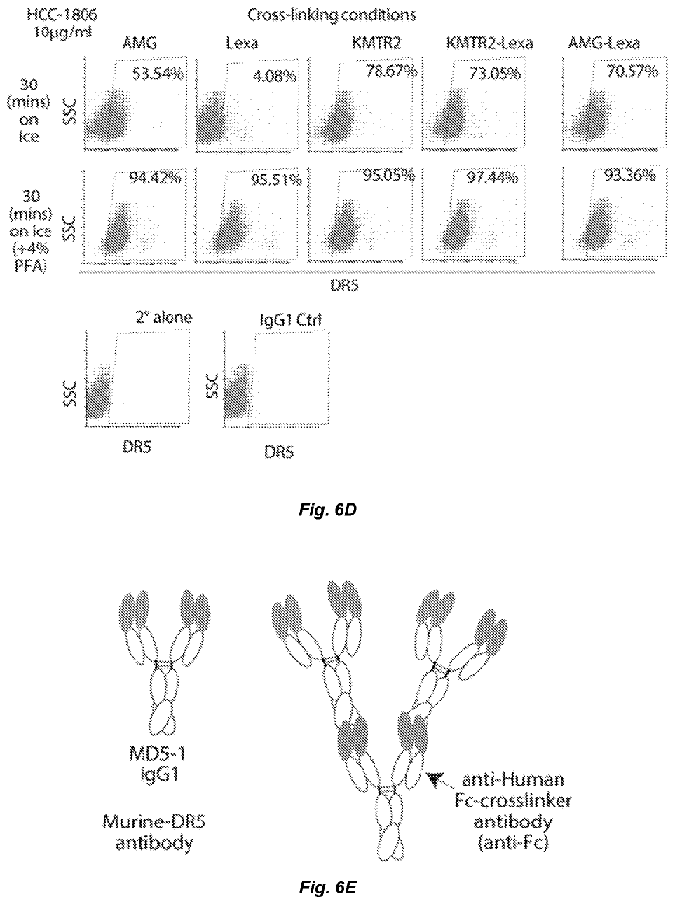

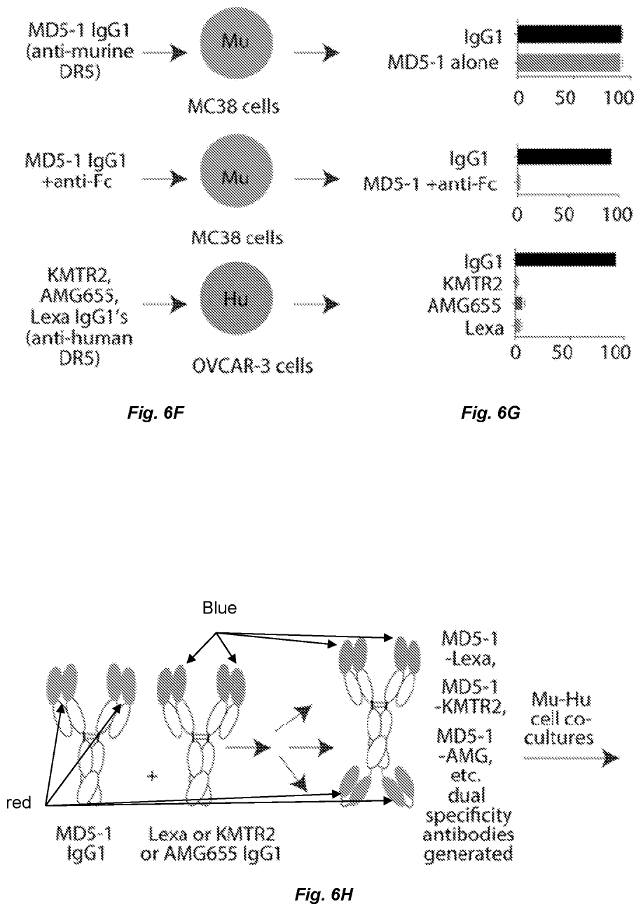

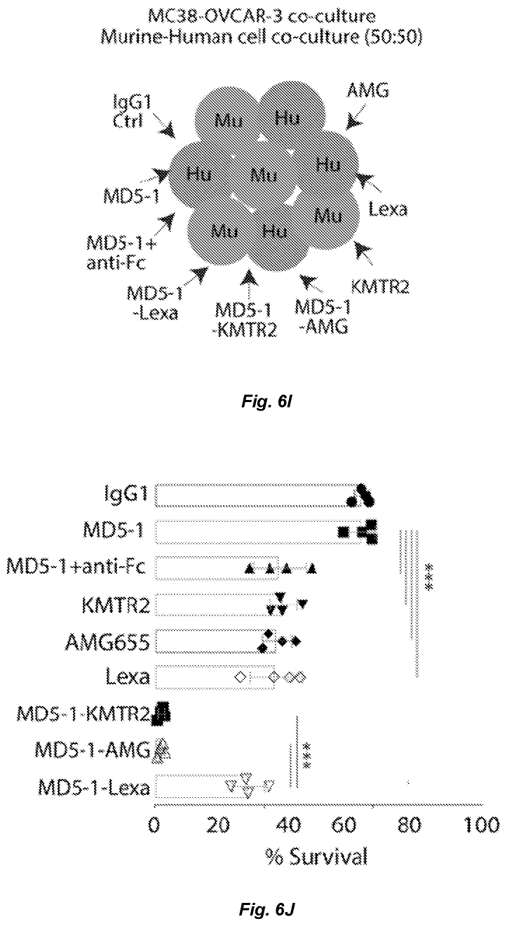

Various high affinity clinical DR5 agonist antibodies (see FIG. 1A, left) that activate caspase-8 and tumor cell death were employed. Surprisingly, despite high-affinity binding against recombinant DR5 in vitro (see the graph, top right of FIG. 1A) and triggering cell death similar to other clinical DR5 agonist antibodies (see FIGS. 1B and 1C), lexatumumab (also referred to herein as “Lexa”) showed almost undetectable binding to surface DR5 in cultured cells (see FIGS. 1D, 1E, and 6A-6D).

Further, in native immunoprecipitation studies, a significantly lower yield of DR5 was pulled down by Lexa as compared to AMG655 (see FIG. 1F).

To reconfirm limited Lexa binding to surface DR5 on tumor cells, the murine DR5 antibody MD5-1 was employed. MD5-1 is a commercially available antibody that is available from Bio X Cell, Lebanon, New Hampshire, United States of America. Unlike human DR5 antibodies, MD5-1 activates tumor cell death of murine cells only if Fc-crosslinked (Shivange et al., 2018; see also FIG. 6E-1G). Lexa and other DR5 antibodies were genetically engineered along with MD5-1 into a dual-specific antibody as described earlier in Shivange et al., 2018; see FIG. 6H). It was hypothesized that due to its significantly lower DR5 binding on human tumor cells, Lexa would not be an effective Fc-crosslinking partner for MD5-1 if murine and human tumor cells were co-cultured (see FIG. 6I).

To test this hypothesis, human-murine tumor cell co-cultures were treated with the MD5-1 containing dual specificity antibodies MD5-1-AMG655 and MD5-1-KMTR2. These dual specificity antibodies completely eliminated human-murine tumor co-cultures, while MD5-1-Lexa was significantly less effective. (see FIG. 6J). These results confirmed the lower differential ability of Lexa as compared to other DR5 agonists to engage DR5 on human tumor cells despite activating effective cell death (see FIG. 1B).

### Example 2

### Lexatumumab Binds Highly Variable CRD3 Domain of DR5

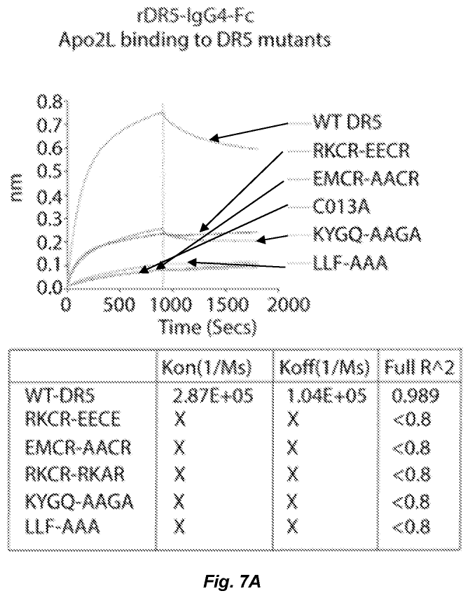

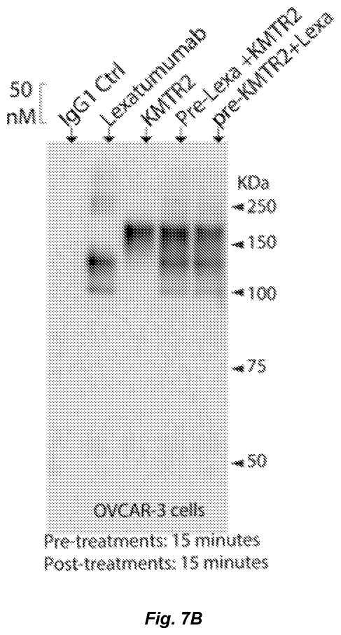

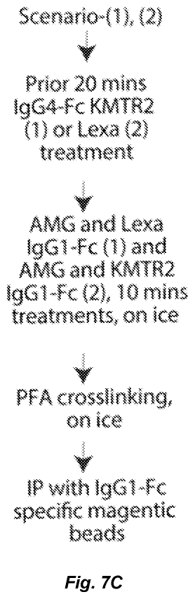

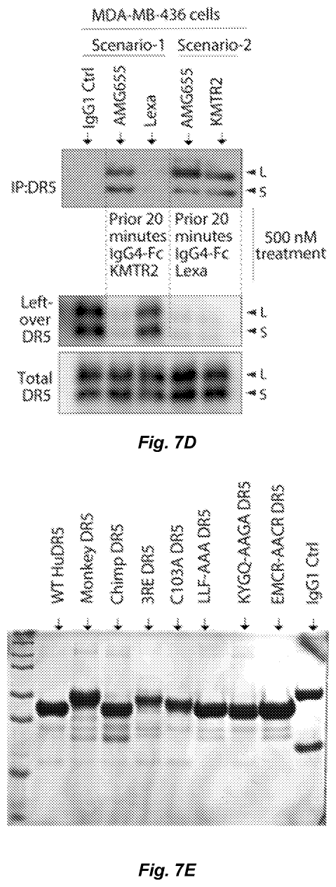

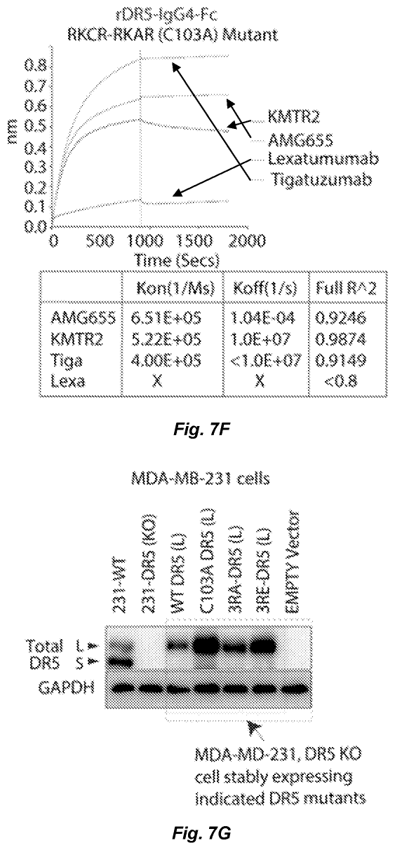

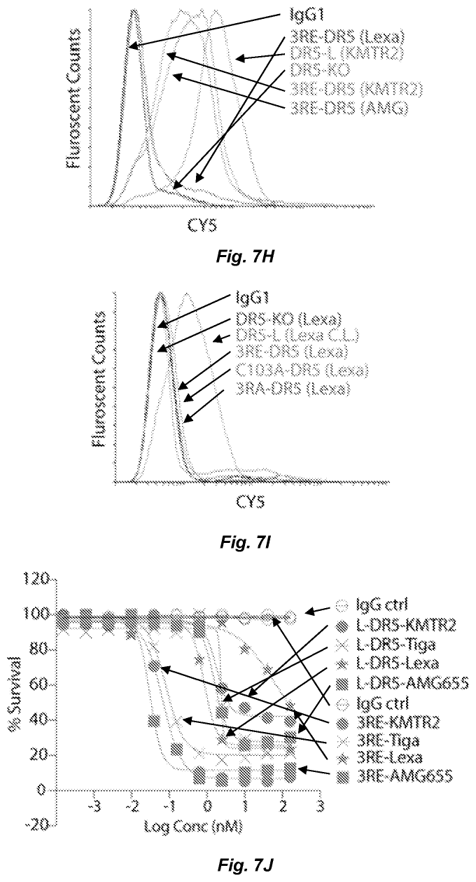

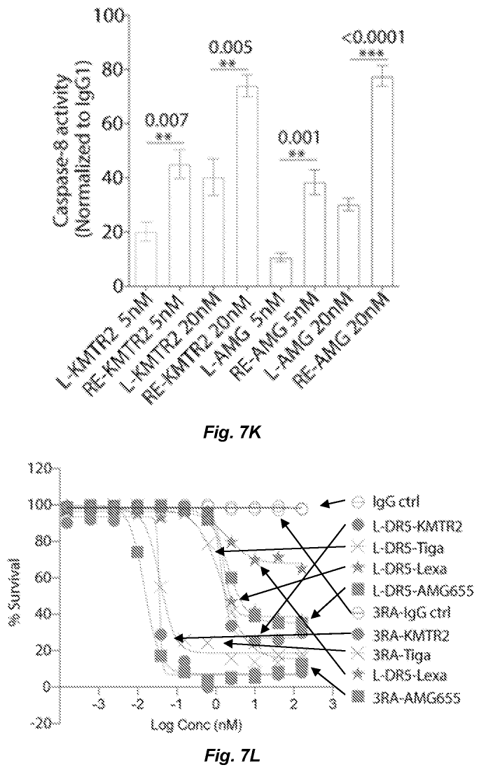

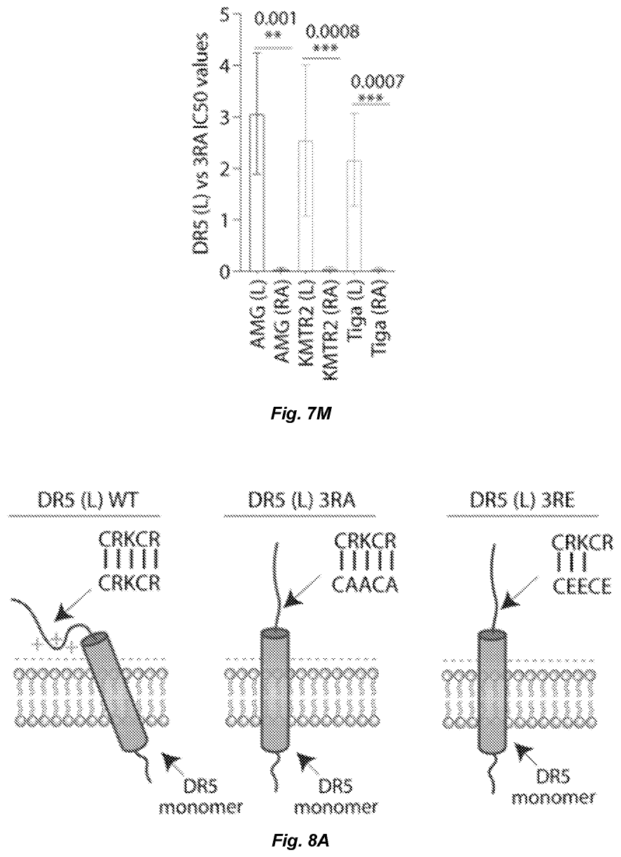

To further investigate the differentially low surface DR5 binding of Lexa, the binding kinetics of various DR5 agonists were analyzed using the ForteBio Octet system. Four different DR5 antibodies alongside Apo2L were tested against IgG4 Fc wild type DR5 and DR5 having mutations in ectodomain. Key DR5 ectodomain mutation regions were chosen (see FIG. 1G) based on previously published and confirmed binding studies of KMTR2 (Tamada et al., 2015), AMG655 (Graves et al., 2014), and Apo2L (Hymowitz et al., 1999; Hymowitz et al., 2000; Mongkolsapaya et al., 1999). An additional 130-137 region of the DR5 ectodomain (i.e., amino acids 185-192 of NP_003833.4, which corresponds to amino acids 130-137 of the mature polypeptide; SEQ ID NO: 76) was used as a non-specific control (see FIG. 1G). When tested using biolayer interferometry (BLI), the binding results confirmed previously published studies (see FIG. 1H). The results were also confirmed with ELISA. Interestingly, Lexa lost binding only against DR5 having mutations in the highly variable CRD3, EMCR-AACR (amino acids 151-165 of NP_003833.4, which correspond to amino acids 96-99 of the mature protein). The same region of DR5 is critical for interactions with two residues (Y214 and Q203 of the mature protein) of Apo2L (Hymowitz et al., 1999; Hymowitz et al., 2000; Mongkolsapaya et al., 1999). Apo2L activates AMG655-mediated DR5 activation cooperatively (Graves et al., 2014) while it interferes with Lexa mediated DR5 apoptotic signaling (Shivange et al., 2018). Thus collectively, these results identified a shared DR5 activation epitope by Lexa and Apo2L. In line with previously published studies (Hymowitz et al., 1999; Hymowitz et al., 2000), Apo2L lost some degree of binding to all generated DR5 ECD mutants, thereby confirming the importance of its multiple low-affinity interactions throughout the DR5 ectodomain (ECD) being responsible for DR5 clustering (FIG. 7A).

### Example 3

### Differential Clustering Profile of DR5 Agonists

Unlike trimeric Apo2L binding to trimeric DR5, agonist antibodies are bivalent. Hence, DR5 antibody mediated clustering and a mechanism therefor remained untested until the presently disclosed subject matter. Thus, to investigate why selective binding of Lexa interferes with its interactions with cell surface DR5, non-reduced (i.e., without β-mercaptoethanol (BME) in lysates) denaturing clustering assays (Valley et al., 2012) were performed. Various DR5 antibodies showed distinct clustering profiles (from each other) when treated side-by-side, supporting their potentially different mechanism of DR5 activation. In particular, only Lexa-treated lysates showed a lower clustering profile with a distinct band of about 100 kDa (see FIGS. 2A-2D and 7B). Neither AMG655 nor KMTR2 generated this particular lower clustering band (FIGS. 2A-2D).

Next, DR5 clustering after pre-KMTR2 (or pre-Lexa) treatments were analyzed. The mixed DR5 clustering profiles of both antibodies were evident on blots at 50 nM non-DR5 saturating concentrations, regardless of the order in which the two DR5 antibodies were added to the cells (see FIGS. 2B and 7B). At a DR5 saturating concentration of 500 nM, a 30-minute pre-treatment of KMTR2 or Tiga inhibited the generation of Lexa-driven DR5 clustering. Particularly, the distinct band of about 100 kDa was not evident. On the other hand, Lexa pre-treatment did not affect the tigatuzumab clustering profile (see FIGS. 2D and 2E; 3rd and 4th lanes). As the 500 nM KMTR2 preincubation step represented saturated DR5 clustering, these results indicated Lexa's inability to engage clustered and activated DR5. In support, KMTR2 pre-treatment blocked DR5 pull down by Lexa, while both KMTR2 and AMG655 maintained binding to both native and activated DR5 (see FIGS. 2F, 2G, 7C, and 7D). Thus, Lexa preferably engaged autoinhibited (i.e., native) ECD as described (Pan et al., 2019), but not the activated clustered DR5. These results also supported the rapid release kinetics mechanism of Lexa following DR5 activation (see FIG. 1).

To further explore Lexa's inability to engage activated DR5, a ribbon trace backbone of DR5 (and DR4) either bound to ligand Apo2L (PDB: 1D0G) or to highly agonist AMG655 (PDB: 4N90), KMTR2 (PDB: 3X3F), apomab (PDB: 40D2) antibodies, or to limitedly agonist BDF1 (PDB: 2H9G), YSD1 (PDB: 1ZA3) antibodies (FIGS. 2H and 2I) was generated. Structurally, both CRD1 and CRD2 maintained similar conformations regardless of being in interface with agonist or non-agonist antibodies (see FIG. 2I). On the other hand, DR5 exhibited significant variations in CRD3 (FIG. 2I), which may adopt an ensemble of conformations. One such confirmation could be an ECD autoinhibited form of DR5 or another one where ECD access in the absence of ligand or agonist antibody binding is incompatible with the formation of the multimeric complex required for signaling (Pan et al., 2019). As Lexa selectively binds to the highly variable DR5 ectodomain in the CRD3 region (see FIGS. 1H, 2H, and 2I), it could potentially represent the ECD autoinhibited form of DR5 as previously described (Pan et al., 2019). Hypothetically, upon DR5 activation, Lexa epitope could potentially structurally be changed (epitope burial in the complex) with the higher-order clustered DR5 column assembly, resulting in its rapid release (FIG. 2J).

### Example 4

### PPCR Charge Substitution Increases Extrinsic Apoptotic Signaling

The DR5 residues in the highly variable DR5 CRD3, next to Apo2L's Y216 and Q205 binding region (Hymowitz et al., 1999), contain an externally exposed patch of positively charged residues (PPCR; amino acids 99-102 of the mature protein; RKCR: 101-104; see FIGS. 2I-2J). The cysteine in the tetrapeptide RKCR (SEQ ID NO: 101) is the third cysteine that forms a disulfide bond with the fifth cysteine (C170 of SEQ ID NO: 62, corresponds to C115 of the mature protein) of CRD3 to generate rigid body looping (Hymowitz et al., 1999; Hymowitz et al., 2000; Mongkolsapaya et al., 1999). The DR5 ectodomain is autoinhibitory by a mechanism that potentially involves either steric hindrance (Endres et al., 2013) or receptor looping away from other DR5 molecules (Endres et al., 2013; Pan et al., 2019).

Whether the PPCR is critical for Lexa binding and works similar to previously described kinase domain harboring KKIK (SEQ ID NO: 131) residues of EGFR for receptor activation (FIGS. 2I and 2J; see also Endres et al., 2013) was also tested. It was hypothesized that stabilized electrostatic interactions of PPCR either with negatively charged membrane domains (Lin et al., 2017) or negatively charged membrane receptors could potentially generate and contribute to autoinhibitory ECD loop formation. The latter constrains neighboring DR5 receptors to initiate oligomerization (FIG. 2J). To test this hypothesis, charge and disulfide bond substituted recombinant DR5 harboring 3RE (RKCR-EECE; i.e., replacement of the RKCR tetrapeptide sequence (SEQ ID NO: 101) with an EECE tetrapeptide sequence (SEQ ID NO: 82)), 3RA (RKCR-AACA; i.e., replacement of the RKCR tetrapeptide sequence (SEQ ID NO: 101) with an AACA tetrapeptide sequence (SEQ ID NO: 134), and C103A (RKCR-RKAR; i.e., replacement of the RKCR tetrapeptide sequence (SEQ ID NO: 101) with an RKAR tetrapeptide sequence (SEQ ID NO: 102)) mutations were generated. The BLI confirmed a significant and complete loss of only Lexa binding to 3RE and C103 mutants, respectively (FIGS. 2K and 7F).

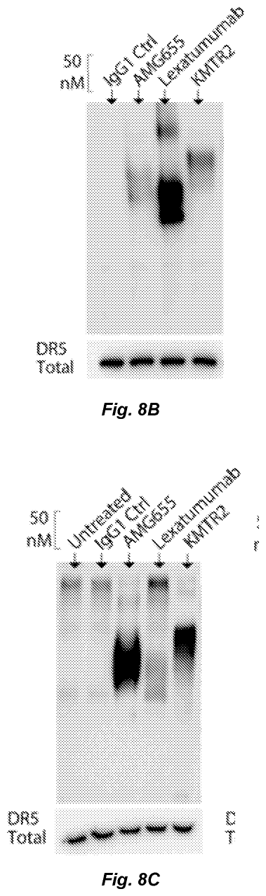

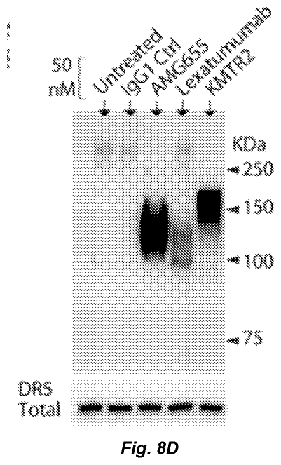

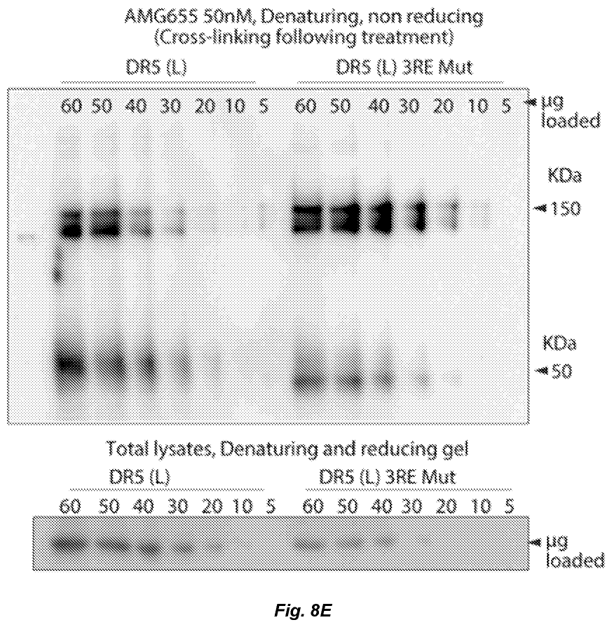

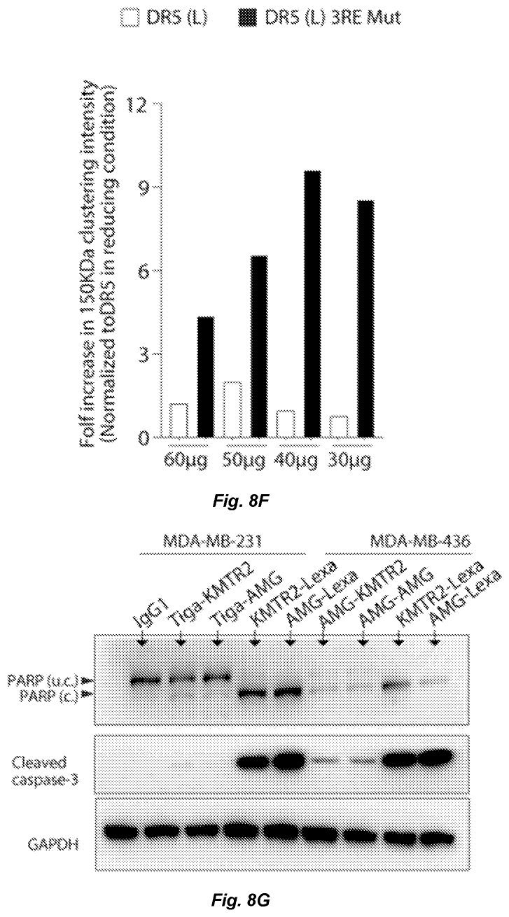

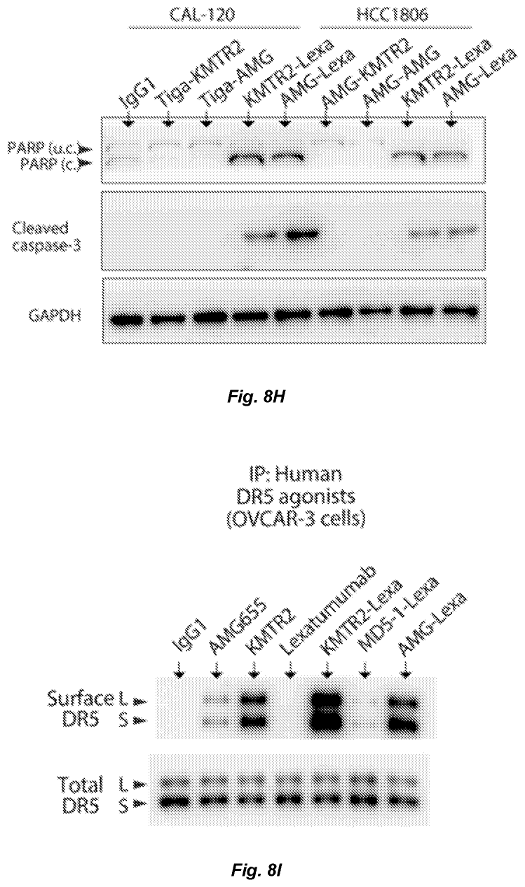

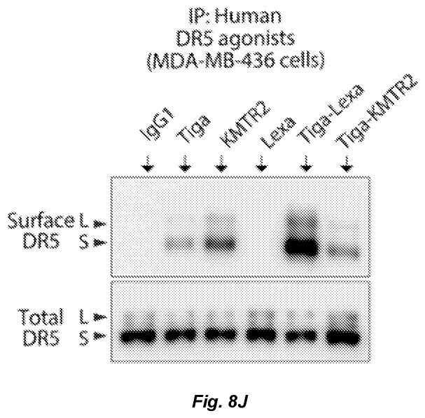

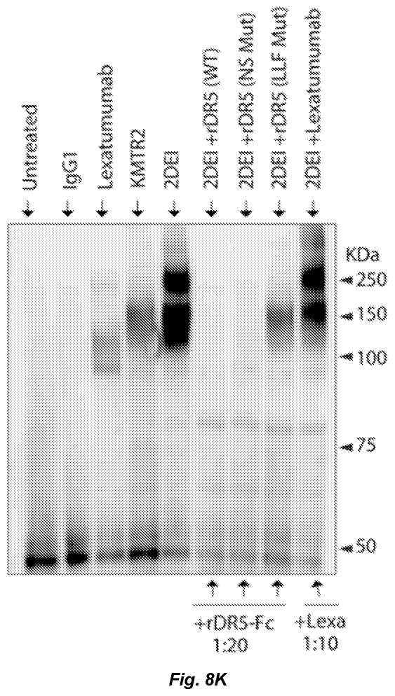

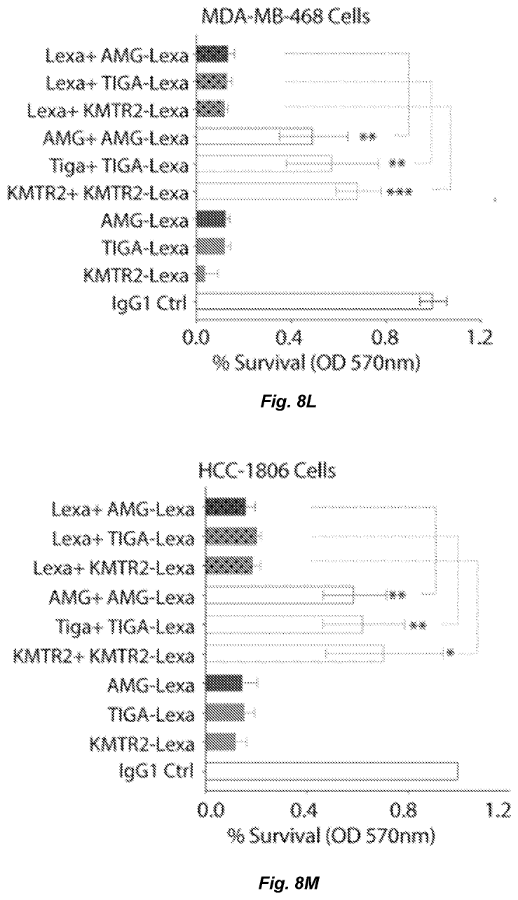

Strikingly, all tested DR5 antibodies (except Lexa) showed significantly higher caspase-8 and cell-killing activity against 3RE and 3RA MDA-MB-231 cells and tumors expressing similar levels of DR5(L) (FIGS. 2M-2O and 8J-8M). When tested, DR5 clustering generated by AMG655 and KMTR2 was higher against 3RE and 3RA stable cells than WT DR5(L) cells (FIGS. 8A-8F). Thus, both PPCR charge substitution (3RA, 3RE mutants) and charge neutralization (by Lexa) served to enhance DR5 clustering and activation. Potential electrostatic looping of PPCR away from the receptor might serve the negative regulatory role of clustering autoinhibition in native DR5. These results indicated the presence of a negative apoptotic regulatory function of PPCR.

### Example 5

### Generation and Activity of 2DEI Antibody

As PPCR-engaging Lexa incorporates the “kiss and run” DR5 binding and activation kinetics (see FIGS. 1 and 2J), the possibility of increasing sustained interference against PPCR autoinhibition function was investigated. To this end, Lexa was genetically linked with CRD-1-2 engaging DR5 antibodies to maintain Lexa's sustained interference of PPCR. This antibody is referred to herein as the Dual DR5 Ectodomain Inhibition (2DEI) targeting strategy (depicted generally in FIGS. 3A and 3B). BLI data confirmed 2DEI (KMTR2-Lexa) binding to two different DR5 epitopes (FIG. 3C). The 2DEI antibody had the highest cytotoxicity gains when tested, while clinical antibodies were not effective against multiple TNBC cell lines (see FIGS. 3D and 3E). Strikingly, in all cases, only PPCR targeting Lexa containing bispecific combinations (KMTR2-Lexa, AMG655-Lexa, Tiga-Lexa) showed the highest cytotoxic activity, caspase-3, poly (ADP-ribose) polymerase (PARP) cleavage, and higher-order clustering. The random bispecific combinations without engaging Lexa's epitope (PPCR) were limitedly effective or ineffective against highly resistant TNBC cells (see FIGS. 3F-3I, 8G, and 8H). Importantly, enhanced death agonism by 2DEI antibody was independent of the bispecific format of antibody. Unlike ineffective bivalent monospecific clinical antibodies, monovalent bispecific 2DEI antibodies also were highly effective in killing tumor cells (see FIGS. 3J-3L). Using combinatorial commercial and clinical DR5 antibodies in a flow cytometry time course experiment, significant overall DR5 internalization by Lexa or 2DEI antibody was not observed (FIG. 3M). Thus, enhanced apoptosis by 2DEI antibody is due to mechanistic targeting of negative regulatory PPCR domain.

As expected, 2DEI antibodies pulled down the highest surface DR5 in native immunoprecipitation (FIGS. 8I and 8J), confirming its ability to maintain Lexa close to the PPCR epitope for the longest time in the higher order complexes. When tested, the 2DEI antibody completely reshuffled the lexatumumab clustering pattern to higher molecular weights regardless of cellular confluency (FIGS. 3I and 8K). Unlike preincubation of recombinant DR5 proteins, 10-fold Lexa preincubation did not change the 2DEI-mediated clustering profile (FIG. 8K) or cell death (FIGS. 8L and 8M). On the other hand, a 10-fold preincubation of KMTR2 or AMG655 significantly interfered with 2DEI-mediated gain of apoptotic function (FIGS. 8L and 8M). Collectively, these results further supported Lexa-IgG1's inability to remain bound to DR5 once activated.

### Example 6

### Anti-tumor Efficacy of 2DEI in Solid Tumors

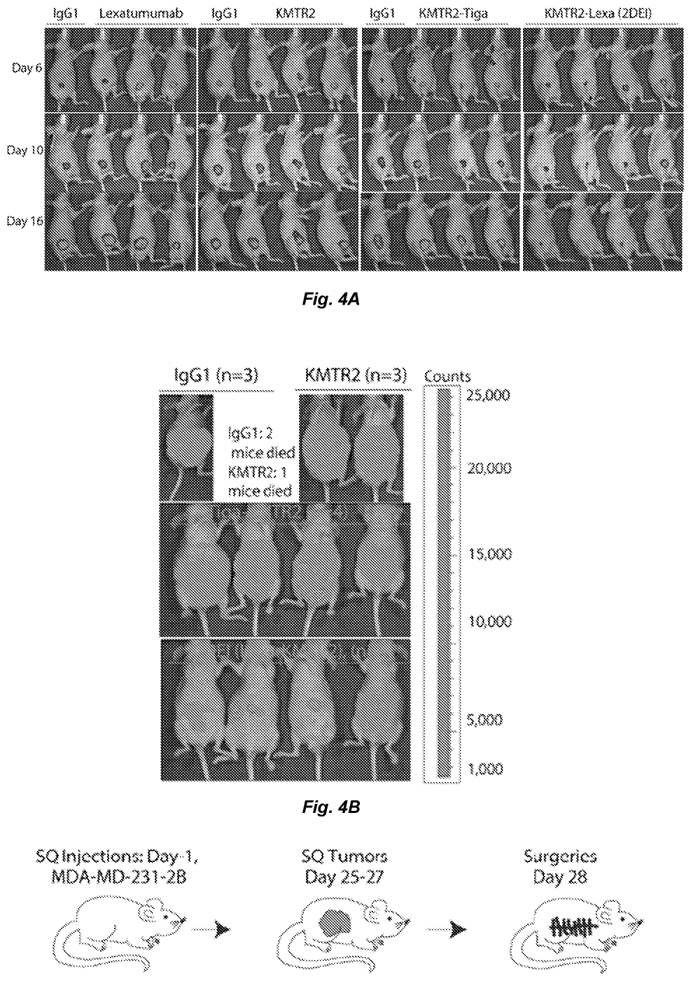

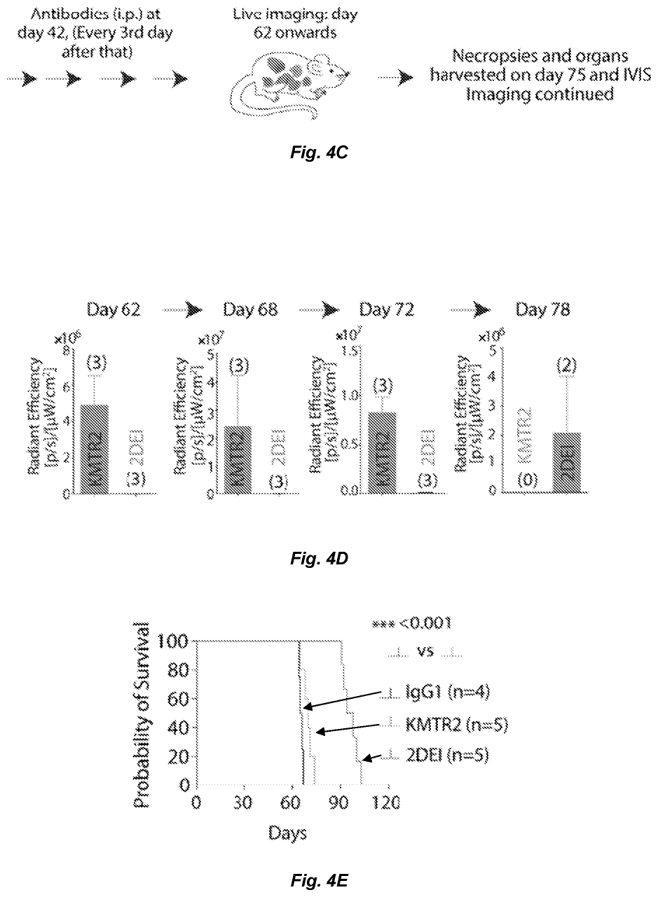

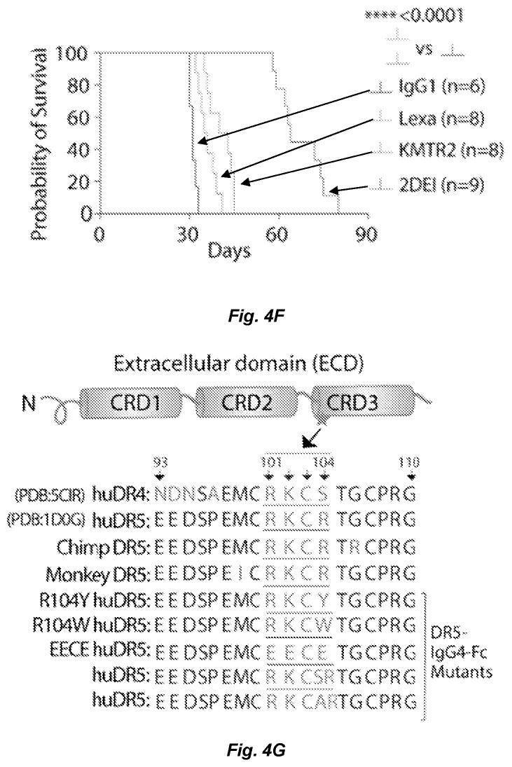

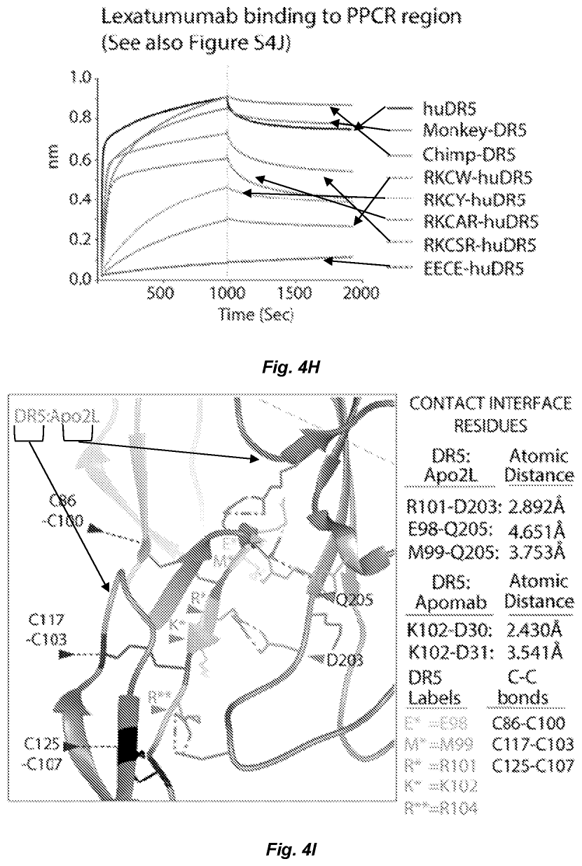

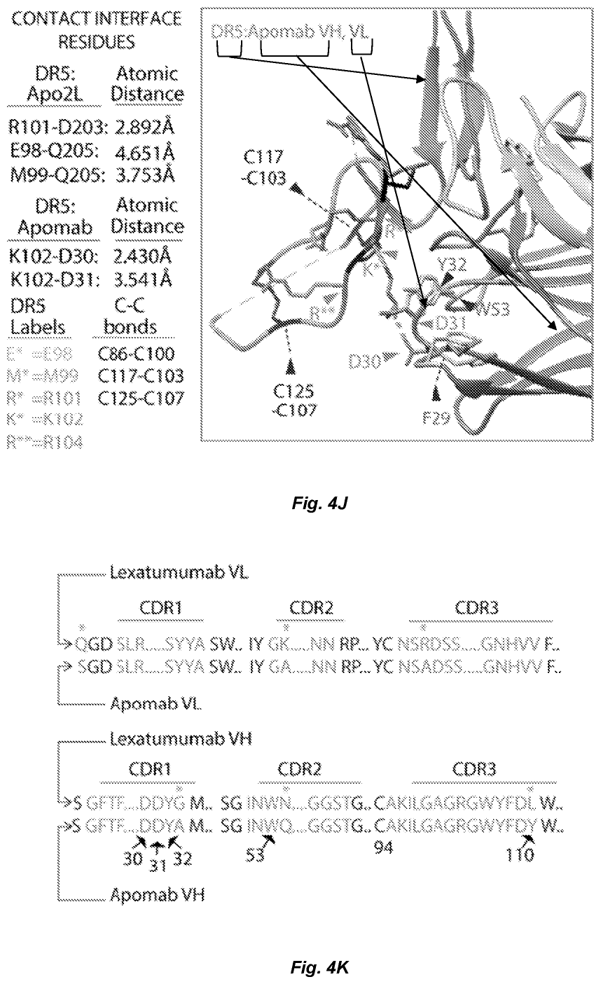

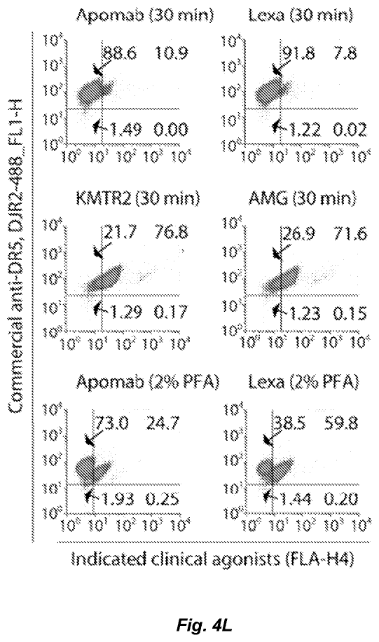

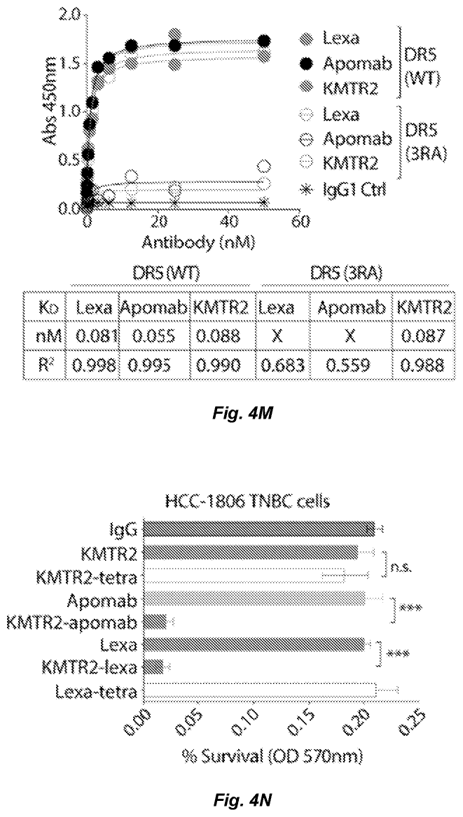

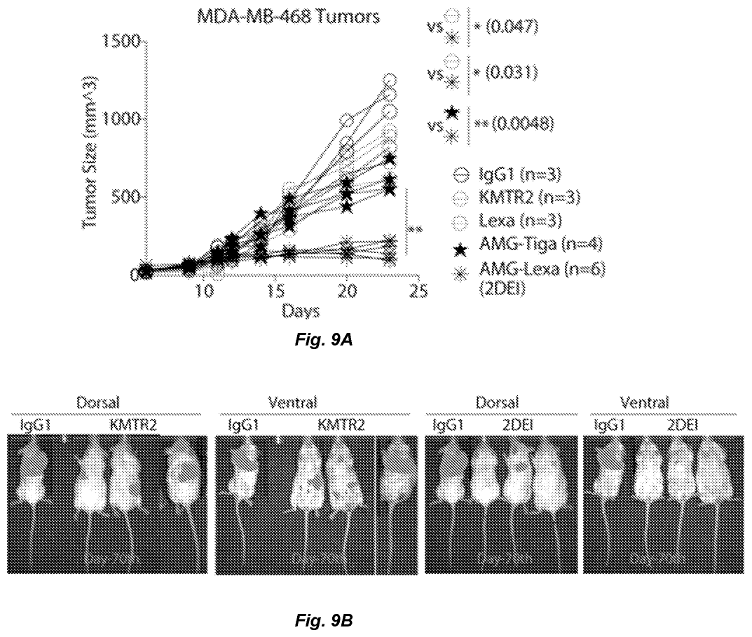

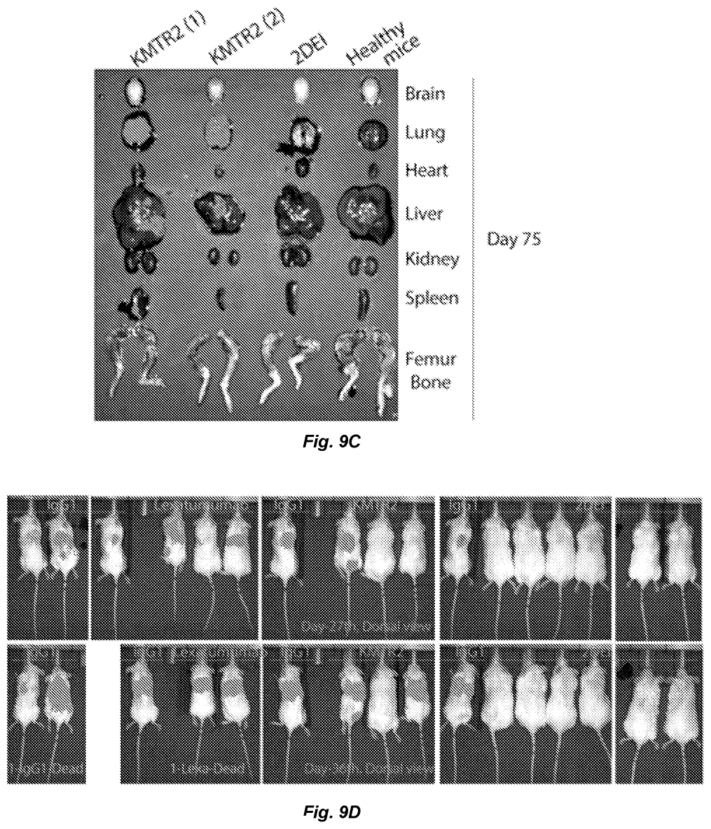

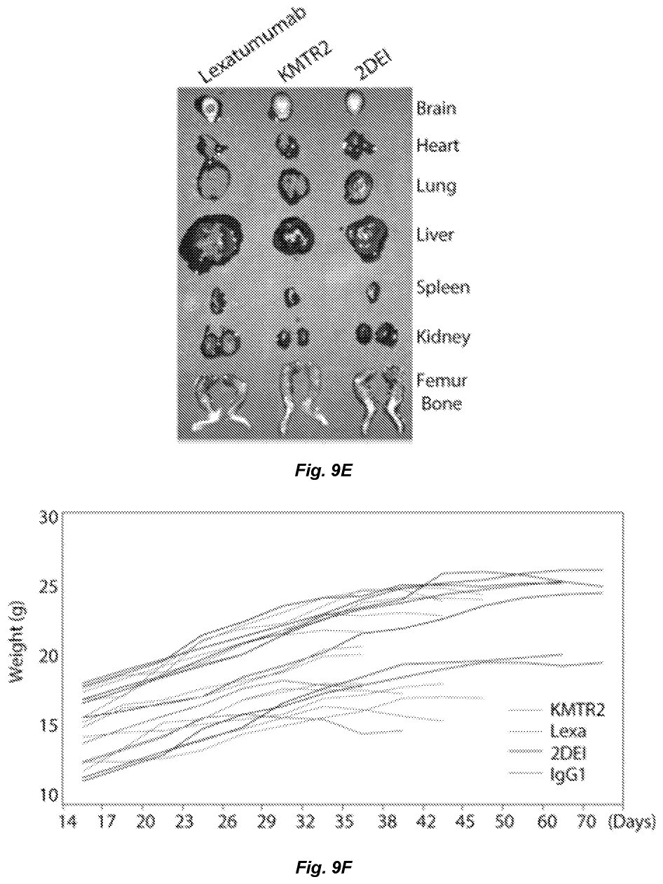

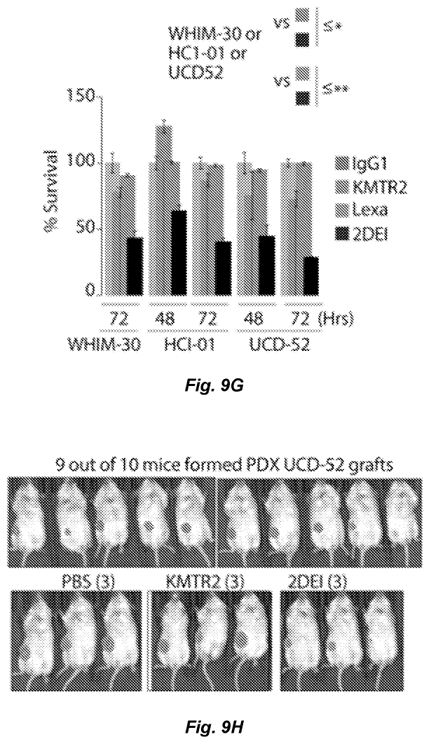

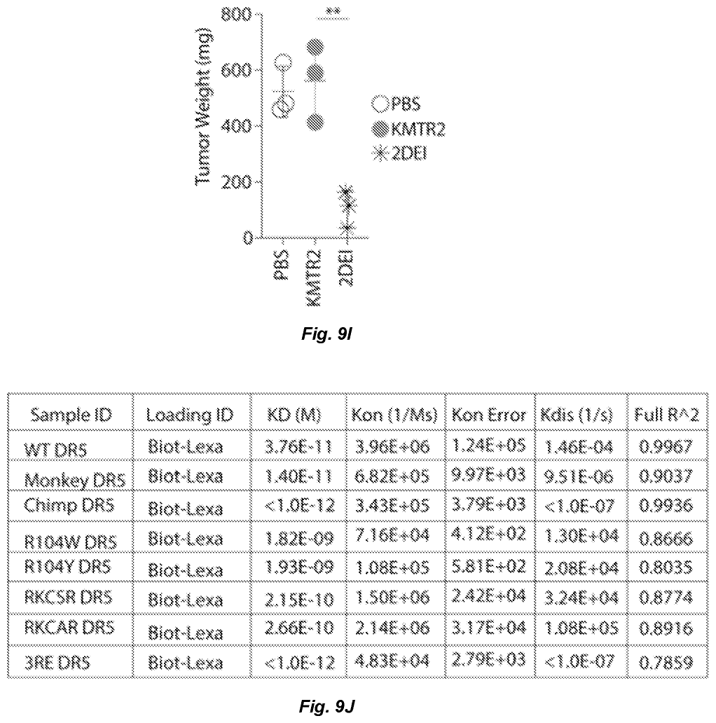

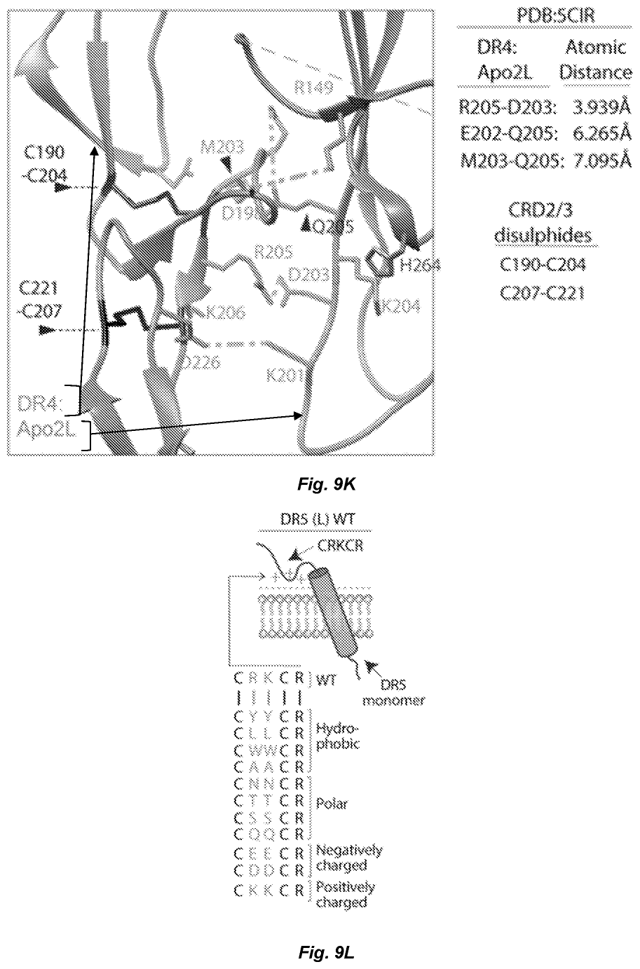

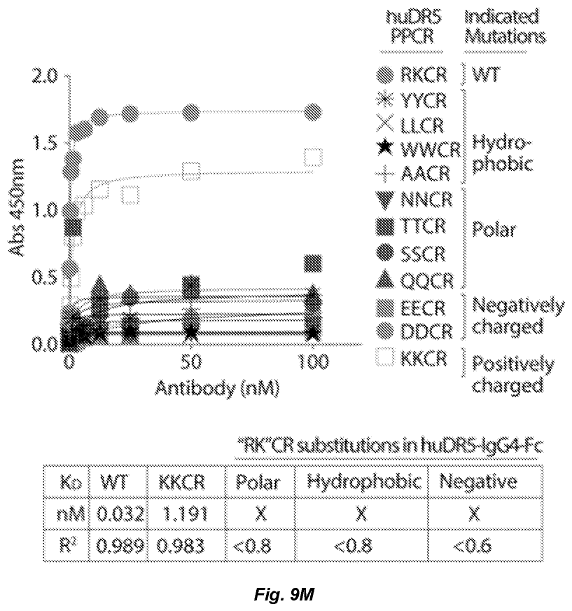

To confirm the apoptotic transition threshold's lowering due to the loss of PPCR function in vivo, low DR5 expressing RFP-stable HCC-1806 (FIG. 3D) breast fat-pad tumor xenografts were employed. Lexa, KMTR2, and Tiga-KMTR2 were completely ineffective, while 2DEI (KMTR2-Lexa) effectively eliminated these tumors (FIG. 4A). Similar, superior efficacy results were seen in intraperitoneal OV90 cells (ovarian) tumors (FIG. 4B) and MDA-MB-468 tumors (FIG. 9A). Since primary TNBC patients respond effectively to surgery and chemotherapy, recurrent tumors were generated in female NSG mice after surgically removing subcutaneous tumors (FIG. 4C) to test the 2DEI antibodies in a more clinically relevant setting. Post-surgery, spontaneous secondary TNBC tumor-bearing animals treated with the 2DEI antibody had significantly reduced tumor burden and survived significantly longer (see FIGS. 4D, 4E, 9B, and 9C).

Next, an aggressive experimental metastatic tumor model (EMTM) originating from the TNBC brain metastatic derivative of human mammary cells called 231-2B cells (Jenkins et al., 2005; Tominaga et al., 2015) was tested. These cells formed highly aggressive tumors and have shown metastases comparable to human TNBC when injected in female NOD SCID gamma (NSG) mice via the intracardiac route (Przanowski et al., 2020). Randomly selected EMTM animals treated with clinical Lexa and KMTR2 antibodies died before day 45. 2DEI injected mice survived an average of 65 days (FIG. 4F) and had reduced tumor loads in animals examined at necropsy (FIGS. 9D and 9E) with insignificant changes in animal weights (FIG. 9F). Similar higher 2DEI efficacy results were seen against TNBC patient-derived spheroid cultures and patient-derived tumor breast fat-pad xenografts (FIGS. 9G-91).

### Example 7

### New 1114 Antibody that Binds PPCR and Works Effectively in 2DEI Antibody

As shown in FIG. 5A and FIG. 5B, CDR sequences of V_{H} (A; SEQ ID NOS: 85-94) and V_{L} (L; SEQ ID NOs: 94-100) were randomly mutated and only antibody clones that expressed at greater than or equal to 0.2 mg/liter were further analyzed for tumor cell killing activity and binding against WT DR5 and 3RE mutant DR5. the percent cell killing of OVCAR-3 cells with V_{H}-V_{L} combinations is shown in FIG. 5C. Only the 1114 antibody killed 100% of ovarian cancer cells.

The sequences of the V_{H} and V_{L} regions of the 1114 antibody are shown in FIG. 5D (SEQ ID NOs: 26 and 27, respectively).

FIG. 5E provides the results of ELISA data of the 1114 antibody against WT and 3RE mutant (PPCR mutant) DR5.

FIG. 5F provides the results of cell killing assays of various antibodies on HCC 1806 cells.

### Discussion of Examples

By making use of multiple clinical DR5 antibodies and Apo2L the negative apoptotic regulatory role of PPCR in the highly variable CRD-3 has been identified. Mutational substitution of PPCR significantly increased tumor cell death implicating PPCR's potential role in the relaxation of DR5 receptors autoinhibitory ECD. As variable protein domains (such as CRD3) are known to undergo the most considerable conformational changes on assembly into complex, the presently disclosed findings of Lexa's potential “kiss and run” working mechanism agrees with the literature (Boehr et al., 2009). Upon Lexa binding, surface exposed PPCR with autoinhibited ECD potentially gets buried in the clustered activated columns of the oligomerized receptor due to conformational clustering (Pan et al., 2019). A similar conformational looping mechanism exists for EGFR activation (Endres et al., 2013). The PPCR patch is identical to the described positively charge patch of KKIK residues in the kinase domain of EGFR, approximately 30 aa away from the juxtamembrane segment. It is involved in looping to interact with the negatively charged plasma membrane at the cytosolic side of cells (Endres et al., 2013). The binding of EGF to EGFR brings a conformational change in the receptor, reliving electrostatic interaction of KKIK (SEQ ID NO: 131) residues with the membrane to allow kinase domain dimerization and activation (Endres et al., 2013). Cancer cells are indeed known to lose polarity due to differential and increased distribution of negatively charged phosphatidylserine, phospholipids, and phosphoproteins on the extracellular side of the membrane (Bernardes & Fialho, 2018). Besides, nearly all high metabolically active cancer cells generate a significant amount of lactate as mobile anions to orchestrate net negative cell surface charge (Chen et al., 2016). Collectively these findings point toward potential stabilized electrostatic interactions of PPCR with the negatively charged membrane domains (Lin et al., 2017). Alternatively, negatively charged membrane receptors or additional steric hindrance mechanisms could be involved in orchestrating the ECD autoinhibitory function to constrain neighboring DR5 receptors to initiate oligomerization.

Lexa engaging PPCR and neighboring cysteine residues in DR4 and DR5 remain broadly preserved in humans and non-human primates (FIGS. 4G, 4H, and 9J; see also Hymowitz et al., 2000; Ramamurthy et al., 2015). Consistent with previous crystal structure studies, among the various low-affinity interactions evenly distributed from upper to lower tip of the DR5 trimer (Cha et al., 2000; Hymowitz et al., 1999; Mongkolsapaya et al., 1999), Apo2L also makes critical contacts near the PPCR region of DR5 and DR4 (FIGS. 4I and 9K). Thus, in retrospect of the negative regulatory role of PPCR, the presently disclosed findings support why nature has selected low-affinity Apo2L as a common DR4/DR5 ligand. Specifically, just above PPCR, Q205 of Apo2L hydrophobically interacts with E98, M99 of DR5, and within PPCR, D203 of Apo2L form ionic interactions with R101 (FIG. 4I) (Hymowitz et al., 1999; Hymowitz et al., 2000). In support, previous studies had described apoptotic competition between PPCR targeting Lexa with Apo2L but not with non-PPCR targeting AMG655 (Graves et al., 2014; Shivange et al., 2018).

Despite numerous CRD-1 and CRD-2 targeting DR5 agonist antibodies, Apomab is the only CRD-3 targeting DR5 antibody described to date (Adams et al., 2008b). Apomab is an effective apoptotic activator. The DR5 binding interface of Apomab V_{H} contains a loop of two critical negatively charged aspartate D30 and D31 residues (structurally stabilized by hydrophobic residues core: of F29, Y32, and W53), which also form a salt bridge with DR5 PPCR, specifically with the K102 (D30-K102: 2.430 Å, PDB: 40D2) of RKCR residues (FIG. 4J). Interestingly, Apomab is ˜95% similar to Lexa with six key substitutions in the complementary determining regions, CDRs (see FIG. 4K; sequence source, PDB: 4OD2). Strikingly, two critical differences in Lexa V_{H} CDRs are right next to structure stabilizing Y30 (A31G) and W51 (Q52N) residues. If these substitutions (in particular Q/N) are involved in differential propensities to alter specific side-chain bonding (Vasudev et al., 2012) of nearby hydrophobic residues to influence D28 and D29 residues interactions with K100 (of RKCR), additional structural studies are needed with Lexa. Nonetheless, unlike KMTR2, similar to Lexa, apomab showed significantly reduced binding to surface DR5 and 3RE mutant DR5 (FIGS. 4L and 4M). Furthermore, saturation mutagenesis of RKCR (SEQ ID NO: 101) with hydrophobic, polar, and negatively charged amino acids eliminated Lexa binding to DR5 (FIGS. 9L and 9M). Significantly genetic construction of apomab scFv in 2DEI also enhanced gain in cytotoxicity (FIG. 4N). These results strongly argue that the proposed positively charge-based anti-clustering ECD auto-inhibitory mechanism is broadly applicable to improve DR5 clustering and activation by all DR5 agonists. Similar to lexatumumab, apomab has also failed to move beyond phase-II trials (Ashkenazi, 2015). Thus, co-targeting anti-PPCR antibodies with CRD-1 (or CRD-2) engaging antibodies via dual-specificity can move death receptor agonism beyond phase-II trials.

How do 2DEI antibodies achieve complete cell killing despite significantly lower DR5 expression in CAL-120, HCC1806, MDA-MB-468, etc., cells? Sustained surface DR5 signaling have been shown to restore sensitivity of DR5 therapy in resistant cells (Jin et al., 2004). Therefore, the 2DEI antibody's potential to remain bound to higher-order DR5 complexes and to increase the surface lifetime of Lexa close to the PPCR epitope is well suited to maintain persistent DR5 activation, despite its lower expression. We must note that a more significant proportion of TNBC and colon cancer patients expresses elevated DR5 levels (Camidge et al., 2007; Forero-Torres et al., 2010b). Despite the latter, phase-II trials of tigatuzumab and apomab as a single agent or a combination of nab-paclitaxel have proven disappointing against TNBC and advanced colon cancer (Camidge et al., 2007; Forero-Torres et al., 2015), If 2DEI can improve survival in TNBC and colon cancer patients, it needs to be seen in clinical trials. In summary, we have discovered the mechanistic insights of limited tumor cytotoxicity by clinical DR5 agonists. Along with factors regulating the varying expression of DR5 (Ashkenazi, 2015), or apoptotic regulators (Spencer et al., 2009) or negatively charged sialylated O-linked glycans within DR5 (Wagner et al., 2007), heterogenous patient tumor cells may also exploit the described mechanism of differential negative charge distribution across the selective membrane domains to interfere with receptor clustering, apoptotic threshold, and clinical resistance.

TABLE 3
List of 2DEI Sequences

| | |
|---|---|
| 2DEI | EVQLVESGGGLVQPGGSLRLSCAASGFTFSSYVMSWVRQ |
| Tigatuzumab-1114 | APGKGLEWVATISSGGSYTYYPDSVKGRFTISRDNAKNTL |
| SEQ ID NO: 1 | YLQMNSLRAEDTAVYYCARRGDSMITTDYWGQGTLVTV |
| | SSASTKGPSVFPLAPSSKSTSGGTAALGCLVKDYFPEPVTV |
| | SWNSGALTSGVHTFPAVLQSSGLYSLSSVVTVPSSSLGTQT |
| | YICNVNHKPSNTKVDKKVEPKSCDKTHTCPPCPAPEAAGG |
| | PSVFLFPPKPKDTLMISRTPEVTCVVVDVSHEDPEVKFNW |
| | YVDGVEVHNAKTKPREEQYNSTYRVVSVLTVLHQDWLN |
| | GKEYKCKVSNKALPAPIEKTISKAKGQPREPQVYTLPPSRE |
| | EMTKNQVSLTCLVKGFYPSDIAVEWESNGQPENNYKTTPP |
| | VLDSDGSFFLYSKLTVDKSRWQQGNVFSCSVMHEALHNH |
| | YTQKSLSLSPGKGGSGGSSSELTQDPAVSVALGQTVRITCQ |
| | GDSLRSYYGSWYQQKPGQAPVLVIYGRNNRPSGIPDRFSG |
| | SSSGNTASLTITGAQAEDEADYYCNSKDSSGNHVVFGGGT |
| | KLTVLGGGGSGGGDSGGGGSGGGGSEVQLVQSGGGVERP |
| | GGSLRLSCAASGFTFDDYSMSWVRQAPGKGLEWVSGINW |
| | NGGSTAYADSVKGRVTISRDNAKNSLYLQMNSLRAEDTA |
| | VYYCAKILGAGRGWYFDAWGKGTTVTVSS |
| 2DEI | QVQLVQSGAEMKKPGASVKVSCKTSGYTFTNYKINWVRQ |
| KMTR2-1114 | APGQGLEWMGWMNPDTDSTGYPQKFQGRVTMTRNTSIST |
| SEQ ID NO: 2 | AYMELSSLRSEDTAVYYCARSYGSGSYYRDYYYGMDVW |
| | GQGTTVTVSSASTKGPSVFPLAPSSKSTSGGTAALGCLVKD |
| | YFPEPVTVSWNSGALTSGVHTFPAVLQSSGLYSLSSVVTVP |
| | SSSLGTQTYICNVNHKPSNTKVDKKVEPKSCDKTHTCPPCP |
| | APEAAGGPSVFLFPPKPKDTLMISRTPEVTCVVVDVSHEDP |
| | EVKFNWYVDGVEVHNAKTKPREEQYNSTYRVVSVLTVLH |
| | QDWLNGKEYKCKVSNKALPAPIEKTISKAKGQPREPQVYT |
| | LPPSREEMTKNQVSLTCLVKGFYPSDIAVEWESNGQPENN |
| | YKTTPPVLDSDGSFFLYSKLTVDKSRWQQGNVFSCSVMHE |
| | ALHNHYTQKSLSLSPGKGGSGGSSSELTQDPAVSVALGQT |
| | VRITCQGDSLRSYYGSWYQQKPGQAPVLVIYGRNNRPSGIP |
| | DRFSGSSSGNTASLTITGAQAEDEADYYCNSKDSSGNHVVF |
| | GGGTKLTVLGGGGSGGGDSGGGGSGGGGSEVQLVQSGGG |
| | VERPGGSLRLSCAASGFTFDDYSMSWVRQAPGKGLEWVS |
| | GINWNGGSTAYADSVKGRVTISRDNAKNSLYLQMNSLRA |
| | EDTAVYYCAKILGAGRGWYFDAWGKGTTVTVSS |
| 2DEI | QVQLQESGPGLVKPSQTLSLTCTVSGGSISSGDYFWSWIRQ |
| AMG655-1114 | LPGKGLEWIGHIHNSGTTYYNPSLKSRVTISVDTSKKQFSLR |
| SEQ ID NO: 3 | LSSVTAADTAVYYCARDRGGDYYYGMDVWGQGTTVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTY |
| | ICNVNHKPSNTKVDKKVEPKSCDKTHTCPPCPAPEAAGGPS |
| | VFLFPPKPKDTLMISRTPEVTCVVVDVSHEDPEVKFNWYV |
| | DGVEVHNAKTKPREEQYNSTYRVVSVLTVLHQDWLNGKE |
| | YKCKVSNKALPAPIEKTISKAKGQPREPQVYTLPPSREEMT |
| | KNQVSLTCLVKGFYPSDIAVEWESNGQPENNYKTTPPVLD |
| | SDGSFFLYSKLTVDKSRWQQGNVFSCSVMHEALHNHYTQ |
| | KSLSLSPGKGGSGGSSSELTQDPAVSVALGQTVRITCQGDS |
| | LRSYYGSWYQQKPGQAPVLVIYGRNNRPSGIPDRFSGSSSG |
| | NTASLTITGAQAEDEADYYCNSKDSSGNHVVFGGGTKLTV |
| | LGGGGSGGGDSGGGGSGGGGSEVQLVQSGGGVERPGGSL |
| | RLSCAASGFTFDDYSMSWVRQAPGKGLEWVSGINWNGGS |
| | TAYADSVKGRVTISRDNAKNSLYLQMNSLRAEDTAVYYC |
| | AKILGAGRGWYFDAWGKGTTVTVSS |
| 2DEI | QVQLVQSGAEMKKPGASVKVSCKTSGYTFTNYKINWVRQ |
| KMTR2-LEXA | APGQGLEWMGWMNPDTDSTGYPQKFQGRVTMTRNTSIST |
| SEQ ID NO: 4 | AYMELSSLRSEDTAVYYCARSYGSGSYYRDYYYGMDVW |
| | GQGTTVTVSSASTKGPSVFPLAPSSKSTSGGTAALGCLVKD |
| | YFPEPVTVSWNSGALTSGVHTFPAVLQSSGLYSLSSVVTVP |
| | SSSLGTQTYICNVNHKPSNTKVDKKVEPKSCDKTHTCPPCP |
| | APEAAGGPSVFLFPPKPKDTLMISRTPEVTCVVVDVSHEDP |
| | EVKFNWYVDGVEVHNAKTKPREEQYNSTYRVVSVLTVLH |
| | QDWLNGKEYKCKVSNKALPAPIEKTISKAKGQPREPQVYT |
| | LPPSREEMTKNQVSLTCLVKGFYPSDIAVEWESNGQPENN |
| | YKTTPPVLDSDGSFFLYSKLTVDKSRWQQGNVFSCSVMHE |
| | ALHNHYTQKSLSLSPGKGGSGGSSSELTQDPAVSVALGQT |
| | VRITCQGDSLRSYYASWYQQKPGQAPVLVIYGKNNRPSGIP |
| | DRFSGSSSGNTASLTITGAQAEDEADYYCNSRDSSGNHVVF |
| | GGGTKLTVLGGGGSGGGDSGGGGSGGGGSEVQLVQSGGG |
| | VERPGGSLRLSCAASGFTFDDYGMSWVRQAPGKGLEWVS |
| | GINWNGGSTGYADSVKGRVTISRDNAKNSLYLQMNSLRA |
| | EDTAVYYCAKILGAGRGWYFDLWGKGTTVTVSS |
| 2DEI | QVQLQESGPGLVKPSQTLSLTCTVSGGSISSGDYFWSWIRQ |
| AMG655-LEXA | LPGKGLEWIGHIHNSGTTYYNPSLKSRVTISVDTSKKQFSLR |
| SEQ ID NO: 5 | LSSVTAADTAVYYCARDRGGDYYYGMDVWGQGTTVTVS |
| | SASTKGPSVFPLAPSSKSTSGGTAALGCLVKDYFPEPVTVS |
| | WNSGALTSGVHTFPAVLQSSGLYSLSSVVTVPSSSLGTQTY |
| | ICNVNHKPSNTKVDKKVEPKSCDKTHTCPPCPAPEAAGGPS |
| | VFLFPPKPKDTLMISRTPEVTCVVVDVSHEDPEVKFNWYV |
| | DGVEVHNAKTKPREEQYNSTYRVVSVLTVLHQDWLNGKE |
| | YKCKVSNKALPAPIEKTISKAKGQPREPQVYTLPPSREEMT |
| | KNQVSLTCLVKGFYPSDIAVEWESNGQPENNYKTTPPVLD |
| | SDGSFFLYSKLTVDKSRWQQGNVFSCSVMHEALHNHYTQ |
| | KSLSLSPGKGGSGGSSSELTQDPAVSVALGQTVRITCQGDS |
| | LRSYYASWYQQKPGQAPVLVIYGKNNRPSGIPDRFSGSSSG |
| | NTASLTITGAQAEDEADYYCNSRDSSGNHVVFGGGTKLTV |
| | LGGGGSGGGDSGGGGSGGGGSEVQLVQSGGGVERPGGSL |
| | RLSCAASGFTFDDYGMSWVRQAPGKGLEWVSGINWNGGS |
| | TGYADSVKGRVTISRDNAKNSLYLQMNSLRAEDTAVYYC |
| | AKILGAGRGWYFDLWGKGTTVTVSS |
| 2DEI | EVQLVESGGGLVQPGGSLRLSCAASGFTFSSYVMSWVRQA |
| Tigatuzumab-LEXA | PGKGLEWVATISSGGSYTYYPDSVKGRFTISRDNAKNTLYL |
| SEQ ID NO: 6 | QMNSLRAEDTAVYYCARRGDSMITTDYWGQGTLVTVSSA |
| | STKGPSVFPLAPSSKSTSGGTAALGCLVKDYFPEPVTVSWN |
| | SGALTSGVHTFPAVLQSSGLYSLSSVVTVPSSSLGTQTYICN |
| | VNHKPSNTKVDKKVEPKSCDKTHTCPPCPAPEAAGGPSVF |
| | LFPPKPKDTLMISRTPEVTCVVVDVSHEDPEVKFNWYVDG |
| | VEVHNAKTKPREEQYNSTYRVVSVLTVLHQDWLNGKEYK |
| | CKVSNKALPAPIEKTISKAKGQPREPQVYTLPPSREEMTKN |
| | QVSLTCLVKGFYPSDIAVEWESNGQPENNYKTTPPVLDSD |
| | GSFFLYSKLTVDKSRWQQGNVFSCSVMHEALHNHYTQKS |
| | LSLSPGKGGSGGSSSELTQDPAVSVALGQTVRITCQGDSLR |
| | SYYASWYQQKPGQAPVLVIYGKNNRPSGIPDRFSGSSSGNT |
| | ASLTITGAQAEDEADYYCNSRDSSGNHVVFGGGTKLTVLG |
| | GGGSGGGDSGGGGSGGGGSEVQLVQSGGGVERPGGSLRL |
| | SCAASGFTFDDYGMSWVRQAPGKGLEWVSGINWNGGSTG |
| | YADSVKGRVTISRDNAKNSLYLQMNSLRAEDTAVYYCAKI |
| | LGAGRGWYFDLWGKGTTVTVSS |

### REFERENCES

All references listed in the instant disclosure, including but not limited to all patents, patent applications and publications thereof, scientific journal articles, and database entries (including but not limited to UniProt, EMBL, and GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Adams et al. (2008a). Structural and functional analysis of the interaction between the agonistic monoclonal antibody Apomab and the proapoptotic receptor DR5. Cell Death and Differentiation 15, 751-761.
Adams et al. (2008b). Structural and functional analysis of the interaction between the agonistic monoclonal antibody Apomab and the proapoptotic receptor DR5. Cell Death Differ 15, 751-761.
Altschul et al. (1990) Basic local alignment search tool. J Mol Biol 215(3): 403-410.
Altschul et al. (1997) Gapped BLAST and PSI-BLAST: a new generation of protein database search programs. Nucleic Acids Res 25(17): 3389-3402.
Ashkenazi (2015). Targeting the extrinsic apoptotic pathway in cancer: lessons learned and future directions. J Clin Invest 125:487-489.
Ashkenazi & Herbst (2008). To kill a tumor cell: the potential of proapoptotic receptor agonists. J Clin Invest 118:1979-1990.
Barbas 3rd. (1995) Synthetic human antibodies. Nat Med 1(8): 837-839.
Bernardes & Fialho (2018) Perturbing the Dynamics and Organization of Cell Membrane Components: A New Paradigm for Cancer-Targeted Therapies. Int J Mol Sci 19(12): 3871.
Bird et al. (1988) Single-chain antigen-binding proteins. Science 242(4877): 423-426.
Boehr et al. (2009) The role of dynamic conformational ensembles in biomolecular recognition. Nat Chem Biol 5:789-796.
Brennan et al. (1985) Preparation of bispecific antibodies by chemical recombination of monoclonal immunoglobulin G1 fragments. Science 229(4708): 81-83.
Burton & Barbas 3rd. (1994) Human antibodies from combinatorial libraries. Adv Immunol 57:191-280.
Camidge et al. (2007) A phase I safety and pharmacokinetic study of apomab, a human DR5 agonist antibody, in patients with advanced cancer. Journal of Clinical Oncology 25(no. 18_suppl) 3582-3582.
Carter et al. (1992a) High level Escherichia coli expression and production of a bivalent humanized antibody fragment. Biotechnology (N Y). 10(2): 163-167.
Carter et al. (1992b) Humanization of an anti-p185HER2 antibody for human cancer therapy. Proc Natl Acad Sci USA 89(10): 4285-4289.
Cha et al. (2000) Crystal structure of TRAIL-DR5 complex identifies a critical role of the unique frame insertion in conferring recognition specificity. J Biol Chem 275:31171-31177.
Chen et al. (2016) Targeting Negative Surface Charges of Cancer Cells by Multifunctional Nanoprobes. Theranostics 6:1887-1898.
Chothia & Lesk (1987) Canonical structures for the hypervariable regions of immunoglobulins. J Mol Biol 196(4): 901-917.
Chou & Fasman (1974) Prediction of protein conformation. Biochemistry 13(2): 222-245.
Chou & Fasman (1978) Empirical predictions of protein conformation. Annu Rev Biochem 47:251-276.
Chou & Fasman (1979) Prediction of beta-turns. Biophys J 26(3): 367-383.
Cole et al. (1985) Antibody production by human X human hybridomas in serum-free medium. J Immunol Methods 78(2): 271-278.
de Kruif et al. (1995) Rapid selection of cell subpopulation-specific human monoclonal antibodies from a synthetic phage antibody library. Proc Natl Acad Sci USA 92(9): 3938-3942.
Deutscher (1990) Maintaining protein stability. Methods Enzymol 182:83-89.
Devereux et al. (1984) A comprehensive set of sequence analysis programs for the VAX. Nucleic Acids Res 12(1 Pt 1): 387-395.
Endres et al. (2013) Conformational coupling across the plasma membrane in activation of the EGF receptor. Cell 152:543-556.
Forero-Torres et al. (2010a) Phase I Trial of Weekly Tigatuzumab, an Agonistic Humanized Monoclonal Antibody Targeting Death Receptor 5(DR5). Cancer Biother Radio 25:13-19.
Forero-Torres et al. (2010b) Phase I trial of weekly tigatuzumab, an agonistic humanized monoclonal antibody targeting death receptor 5(DR5). Cancer Biother Radiopharm 25:13-19.
Forero-Torres et al. (2015) TBCRC 019: A Phase II Trial of Nanoparticle Albumin-Bound Paclitaxel with or without the Anti-Death Receptor 5 Monoclonal Antibody Tigatuzumab in Patients with Triple-Negative Breast Cancer. Clin Cancer Res 21:2722-2729.
Graves et al. (2014). Apo2L/TRAIL and the death receptor 5 agonist antibody AMG 655 cooperate to promote receptor clustering and antitumor activity. Cancer Cell 26:177-189.
Green & Sambrook J (2012) Molecular Cloning: A Laboratory Manual. 4th Edition, Cold Spring Harbor Laboratory Press, Cold Spring Harbor, New York, United States of America.
Harlow & Lane (1988) Antibodies: A Laboratory Manual. Cold Spring Harbor Laboratory Press, Cold Spring Harbor, New York, United States of America.
Huet et al. (2012) Abstract 3853: TAS266, a novel tetrameric nanobody agonist targeting death receptor 5(DR5), elicits superior antitumor efficacy than conventional DR5-targeted approaches. Cancer Res 72(8 Supplement): 3853.
Huse et al. (1992) Generation of a large combinatorial library of the immunoglobulin repertoire in phage lambda. 1989. Biotechnology 24:517-523.
Huston et al. (1988) Protein engineering of antibody binding sites: recovery of specific activity in an anti-digoxin single-chain Fv analogue produced in Escherichia coli. Proc Natl Acad Sci USA 85(16): 5879-5883.
Hymowitz et al. (1999) Triggering cell death: the crystal structure of Apo2L/TRAIL in a complex with death receptor 5. Mol Cell 4:563-571.
Hymowitz et al. (2000) A unique zinc-binding site revealed by a high-resolution X-ray structure of homotrimeric Apo2L/TRAIL. Biochemistry 39:633-640.
Jenkins et al. (2005) Bioluminescent human breast cancer cell lines that permit rapid and sensitive in vivo detection of mammary tumors and multiple metastases in immune deficient mice. Breast Cancer Res 7: R444-454.
Jin et al. (2004) Deficient tumor necrosis factor-related apoptosis-inducing ligand (TRAIL) death receptor transport to the cell surface in human colon cancer cells selected for resistance to TRAIL-induced apoptosis. J Biol Chem 279:35829-35839.
Johnson et al. (2003) Human agonistic anti-TRAIL receptor antibodies, HGS-ETR1 and HGS-ETR2, induce apoptosis in diverse hematological tumor lines. Blood 102: 891a-891a.
Jones et al. (1986) Replacing the complementarity-determining regions in a human antibody with those from a mouse. Nature 321(6069): 522-525.
Kaplan-Lefko et al. (2010) Conatumumab, a fully human agonist antibody to death receptor 5, induces apoptosis via caspase activation in multiple tumor types. Cancer Biol Ther 9:618-631.
Karlin & Altschul (1990) Methods for assessing the statistical significance of molecular sequence features by using general scoring schemes. Proc Natl Acad Sci USA 87(6): 2264-2268.
Karlin & Altschul (1993) Applications and statistics for multiple high-scoring segments in molecular sequences. Proc Natl Acad Sci USA 90(12): 5873-5877.
Kozbor et al. (1983) A comparative analysis of the phenotypic characteristics of available fusion partners for the construction of human hybridomas. Hybridoma 2(1): 7-16.
Kyte & Doolittle (1982) A simple method for displaying the hydropathic character of a protein. J Mol Biol 157(1): 105-132.
Lin et al. (2017) Lipid rafts sense and direct electric field-induced migration. Proc Natl Acad Sci US A 114:8568-8573.
Marks et al. (1991) By-passing immunization. Human antibodies from V-gene libraries displayed on phage. J Mol Biol 222(3): 581-597.
Mongkolsapaya et al. (1999) Structure of the TRAIL-DR5 complex reveals mechanisms conferring specificity in apoptotic initiation. Nat Struct Biol 6:1048-1053.
Morimoto & Inouye (1992) Single-step purification of F(ab′)2 fragments of mouse monoclonal antibodies (immunoglobulins G1) by hydrophobic interaction high performance liquid chromatography using TSKgel Phenyl-5 PW. J Biochem Biophys Methods 24(1-2): 107-117.
Morrison et al. (1984) Chimeric human antibody molecules: mouse antigen-binding domains with human constant region domains. Proc Natl Acad Sci USA 81(21): 6851-6855.
Motoki et al. (2005) Enhanced apoptosis and tumor regression induced by a direct agonist antibody to tumor necrosis factor-related apoptosis-inducing ligand receptor 2. Clin Cancer Res 11:3126-3135.
Nasoff et al. (19997) Cloning and expression of the human tumor-specific antibody GM4. Hybridoma 16(5): 427-439.
Neuberger et al. (1984) Recombinant antibodies possessing novel effector functions. Nature 312(5995): 604-608.
Pan et al. (2019) Higher-Order Clustering of the Transmembrane Anchor of DR5 Drives Signaling. Cell 176:1477-1489.
PCT International Patent Application Publication Nos. WO 1992/02190; WO 1993/16185.
Presta (1992) Antibody engineering. Curr Opin Biotechnol 3(4): 394-398.
Presta et al. (1993) Humanization of an antibody directed against IgE. J Immunol 151(5): 2623-2632.
Przanowski et al. (2020) Oncogenic TRIM37 links chemoresistance and metastatic fate in triple-negative breast cancer. Cancer Res 80(21): 4791-4804.
Ramamurthy et al. (2015) The structure of the death receptor 4-TNF-related apoptosis-inducing ligand (DR4-TRAIL) complex. Acta Crystallogr F Struct Biol Commun 71:1273-1281.
Riechmann et al. (1988) Reshaping human antibodies for therapy. Nature 332(6162): 323-327.
Shivange et al. (2018) A Single-Agent Dual-Specificity Targeting of FOLR1 and DR5 as an Effective Strategy for Ovarian Cancer. Cancer Cell 34:331-345.
Sims et al. (1993) A humanized CD18 antibody can block function without cell destruction. J Immunol 151(4): 2296-2308.
Spencer et al. (2009) Non-genetic origins of cell-to-cell variability in TRAIL-induced apoptosis. Nature 459:428-432.
Takeda et al. (1985) Construction of chimaeric processed immunoglobulin genes containing mouse variable and human constant region sequences. Nature 314(6010): 452-454.
Tamada et al. (2015) TRAIL-R2 Superoligomerization Induced by Human Monoclonal Agonistic Antibody KMTR2. Sci Rep 5:17936.
Tominaga et al. (2015) Brain metastatic cancer cells release microRNA-181c-containing extracellular vesicles capable of destructing blood-brain barrier. Nat Commun 6:6716.
Tuszynski et al. (1988) Thrombospondin promotes platelet aggregation. Blood 72(1): 109-115.
U.S. Patent Application Publication Nos. 2002/0034765; 2003/0017534; 2003/0022244; 2003/0153043; 2004/0253645; 2006/0073137; 2018/0298087; 2018/0312588; 2018/0346564; 2019/0151448.
U.S. Pat. Nos. 4,554,101; 4,816,567; 4,816,567; 4,946,778; 4,975,369; 5,001,065; 5,075,431; 5,081,235; 5,169,939; 5,202,238; 5,204,244; 5,225,539; 5,231,026; 5,292,867; 5,354,847; 5,436,157; 5,436,157; 5,472,693; 5,482,856; 5,482,856; 5,482,856; 5,482,856; 5,491,088; 5,500,362; 5,502,167; 5,530,101; 5,571,894; 5,585,089; 5,587,458; 5,641,870; 5,693,761; 5,693,762; 5,712,120; 5,714,350; 5,766,886; 5,770,196; 5,777,085; 5,821,123; 5,821,337; 5,869,619; 5,877,293; 5,886,152; 5,895,205; 5,929,212; 6,054,297; 6,180,370; 6,407,213; 6,479,284; 6,548,640; 6,632,927; 6,639,055; 6,677,436; 6,750,325; 6,797,492; 7,060,808; 7,906,625; 8,398,980; 8,436,150; 8,796,439; 10,253,111.
Valley et al. (2012) Tumor necrosis factor-related apoptosis-inducing ligand (TRAIL) induces death receptor 5 networks that are highly organized. J Biol Chem 287:21265-21278.
Vasudev et al. (2012) Asparagine and glutamine differ in their propensities to form specific side chain-backbone hydrogen bonded motifs in proteins. Proteins 80:991-1002.
Verhoeyen & Riechmann (1988) Engineering of antibodies. Bioessays 8(2): 74-78.
Wagner et al. (2007) Death-receptor O-glycosylation controls tumor-cell sensitivity to the proapoptotic ligand Apo2L/TRAIL. Nat Med 13:1070-1077.
Wajant (2019) Molecular Mode of Action of TRAIL Receptor Agonists-Common Principles and Their Translational Exploitation. Cancers (Basel) 11(7): 954.
Winter & Milstein (1991) Man-made antibodies. Nature 349(6307): 293-299.
Wright et al. (1992) Genetically engineered antibodies: progress and prospects. Crit Rev Immunol 12 (3-4): 125-168.

### Additional Exemplary Sequences

Human tumor necrosis factor receptor superfamily member 10B isoform 1 precursor (also known as DR5 isoform 1 precursor); GENBANK® Accession No. NP_003833.4 (SEQ ID NO: 62)

MEQRGQNAPA ASGARKRHGP GPREARGARP GPRVPKTLVL VVAAVLLLVS
AESALITQQD LAPQQRAAPQ QKRSSPSEGL CPPGHHISED GRDCISCKYG
QDYSTHWNDL LFCLRCTRCD SGEVELSPCT TTRNTVCQCE EGTFREEDSP
EMCRKCRTGC PRGMVKVGDC TPWSDIECVH KESGTKHSGE VPAVEETVTS
SPGTPASPCS LSGIIIGVTV AAVVLIVAVF VCKSLLWKKV LPYLKGICSG
GGGDPERVDR SSQRPGAEDN VLNEIVSILQ PTQVPEQEME VQEPAEPTGV
NMLSPGESEH LLEPAEAERS QRRRLLVPAN EGDPTETLRQ CFDDFADLVP
FDSWEPLMRK LGLMDNEIKV AKAEAAGHRD TLYTMLIKWV NKTGRDASVH
TLLDALETLG ERLAKQKIED HLLSSGKEMY LEGNADSAMS

Human tumor necrosis factor receptor superfamily member 10B isoform 2 precursor (also known as DR5 isoform 2 precursor); GENBANK® Accession No. NP_671716.2 (SEQ ID NO: 63)

MEQRGQNAPA ASGARKRHGP GPREARGARP GPRVPKTLVL VVAAVLLLVS
AESALITQQD LAPQQRAAPQ QKRSSPSEGL CPPGHHISED GRDCISCKYG
QDYSTHWNDL LFCLRCTRCD SGEVELSPCT TTRNTVCQCE EGTFREEDSP
EMCRKCRTGC PRGMVKVGDC TPWSDIECVH KESGIIIGVT VAAVVLIVAV
FVCKSLLWKK VLPYLKGICS GGGGDPERVD RSSQRPGAED NVLNEIVSIL
QPTQVPEQEM EVQEPAEPTG VNMLSPGESE HLLEPAEAER SQRRRLLVPA
NEGDPTETLR QCFDDFADLV PFDSWEPLMR KLGLMDNEIK VAKAEAAGHR
DTLYTMLIKW VNKTGRDASV HILLDALETL GERLAKQKIE DHLLSSGKFM
YLEGNADSAM S

It will be understood that various details of the presently disclosed subject matter can be changed without departing from the scope of the presently disclosed subject matter. Furthermore, the foregoing description is for the purpose of illustration only, and not for the purpose of limitation.

1. A method for treating a cancer in a subject in need thereof, the method comprising, consisting essentially of, or consisting of administering to the subject:
(a) a first composition comprising an effective amount of a first binding agent that selectively binds to a human cysteine-rich domain 3 (CRD3) of DR5, optionally an RKCR (SEQ ID NO: 101) peptide motif present in a human cysteine-rich domain 3 (CRD3) of DR5, further optionally a DR5 comprising an amino acid sequence as set forth in SEQ ID NO: 62 or SEQ ID NO: 63); and
(b) a second composition comprising, consisting essentially of, or consisting of an effective amount of a second binding agent, wherein the second binding agent selectively binds to a binding partner selected from the group consisting of a human cysteine-rich domain 1 (CRD1), a cysteine-rich domain 2 (CRD2), and a tumor-associated antigen, optionally wherein the second binding agent comprises a DR5 agonist.

2. The method of claim 1, wherein the cancer comprises a solid tumor.

3. The method of claim 1, wherein the first binding agent, the second binding agent, or both comprise an antibody.

4. The method of claim 3, wherein the antibody is selected from the group consisting of lexatumumab and antibody 1114, wherein antibody 1114 comprises a heavy chain comprising an amino acid sequence as set forth in FIG. 5A, optionally an amino acid sequence as set forth in any one of SEQ ID NOs: 85-94, a light chain comprising an amino acid sequence as set forth in FIG. 5B, optionally an amino acid sequence as set forth in any one of SEQ ID NOs: 95-100, a biologically active fragment thereof, a homolog thereof, or any combination thereof.

5. The method of claim 1, wherein the second binding agent selectively binds to at least one of a CRD1 and a CRD2 of DR5, and further wherein the second binding agent has DR5 agonist activity.

6. The method of claim 5, wherein the second binding agent comprises an antibody, optionally an antibody with DR5 agonist activity.

7. The method of claim 1, wherein the first composition and the second composition are provided as a single composition.

8. The method of claim 7, wherein the single composition comprises a bispecific antibody.

9. The method of claim 7, wherein the single composition comprises a 2DEI antibody, optionally wherein the 2DEI antibody comprises a sequence as set forth in Table 3, optionally any one of SEQ ID NOs: 1-6, or a biologically active fragment and/or homolog thereof.

10. The method of claim 7, wherein the single composition comprises an antibody selected from the group comprising lexatumumab and antibody 1114, wherein antibody 1114 comprises a heavy chain comprising an amino acid sequence as set forth in FIG. 5A, optionally any one of SEQ ID NOs: 85-94, a light chain comprising an amino acid sequence as set forth in FIG. 5B, optionally any one of SEQ ID NOs: 95-100, a biologically active fragment thereof, a homolog thereof, or any combination thereof.

11. A composition comprising:
(a) an effective amount of a first binding agent that selectively binds to a human cysteine-rich domain 3 (CRD3) of DR5, optionally that selectively binds an RKCR (SEQ ID NO: 101) peptide motif present in a human cysteine-rich domain 3 (CRD3) of DR5, optionally a DR5 comprising an amino acid sequence as set forth in SEQ ID NO: 62 or SEQ ID NO: 63); and
(b) an effective amount of a second binding agent that selectively binds to a human cysteine-rich domain 1 (CRD1), a cysteine-rich domain 2 (CRD2), or a tumor-associated antigen, optionally wherein the second binding agent has DR5 agonist activity.

12. The composition of claim 11, wherein the composition comprises a bispecific antibody.

13. The composition of claim 11, wherein the composition comprises a 2DEI antibody, optionally wherein the 2DEI antibody comprises an amino acid sequence as set forth in Table 3, optionally an amino acid sequence as set forth in any one of SEQ ID NOs: 1-6, or a biologically active fragment or homolog thereof.

14. The composition of claim 11, further comprising a pharmaceutically acceptable carrier, optionally a pharmaceutically acceptable carrier that is pharmaceutically acceptable for use in a human.

15. A bispecific antibody that binds to a death receptor 5 (DR5) polypeptide, wherein the bispecific antibody comprises a first antigen binding moiety that is specific for an RKCR (SEQ ID NO: 101) tetrapeptide motif of a human CRD3 of DR5 and a second antigen binding moiety that is specific for an epitope of DR5 that is distinct from the RKCR (SEQ ID NO: 101) tetrapeptide motif or a tumor-associated antigen, optionally wherein the second antigen binding moiety has DR5 agonist activity.

16. The bispecific antibody of claim 15, wherein the first binding moeity or the second binding moeity is an antibody selected from the group consisting of AMG655, KMTR2, Tigatuzumab, lexatumumab, apomab, and antibody 1114, wherein antibody 1114 selectively binds to an RKCR (SEQ ID NO: 101) tetrapeptide motif present in a human cysteine-rich domain 3 (CRD3) of DR5, optionally a DR5 comprising an amino acid sequence as set forth in SEQ ID NO: 62 or SEQ ID NO: 63, or is a biologically active fragment or homolog thereof.

17. The bispecific antibody of claim 15, wherein the first binding moiety or the second binding moiety comprises a 2DEI antibody and/or a biologically active fragment or derivative thereof, optionally wherein the 2DEI antibody and/or the biologically active fragment or derivative thereof comprises an amino acid sequence as set forth in Table 3, optionally an amino acid sequence as set forth in any one of SEQ ID NOs: 1-6.

18. The bispecific antibody of claim 15, wherein the antibody is humanized.

19. The bispecific antibody of claim 15, further comprising a pharmaceutically acceptable carrier, optionally a pharmaceutically acceptable carrier that is pharmaceutically acceptable for use in a human.

20. An antibody that binds to a death receptor 5 (DR5) polypeptide, wherein the antibody comprises an antigen binding site that binds to a human CRD3, optionally to an RKCR (SEQ ID NO: 101) tetrapeptide motif of a human CRD3 of DR5, further optionally wherein the antibody is antibody 1114, wherein antibody 1114 comprises a heavy chain comprising an amino acid sequence as set forth in SEQ ID NO: 26 and a light chain comprising an amino acid sequence as set forth in SEQ ID NO: 27, a biologically active fragment thereof, a homolog thereof, or any combination thereof.

21. The antibody of claim 20, wherein the antibody is humanized.

22. The antibody of claim 20, further comprising a pharmaceutically acceptable carrier, optionally a pharmaceutically acceptable carrier that is pharmaceutically acceptable for use in a human.

23. The antibody of claim 20, wherein the antibody is a bispecific antibody, optionally a bispecific antibody that comprises a binding arm that binds to a tumor-associated antigen.

24. A method for activating a DR5 biological activity in a cell, tissue, or organ, optionally a cell, tissue, or organ present in a subject, the method comprising, consisting essentially of, or consisting of administering to the subject:
(a) a first composition comprising an effective amount of a first binding agent that selectively binds to a human cysteine-rich domain 3 (CRD3) of DR5, optionally an RKCR (SEQ ID NO: 101) peptide motif present in a human cysteine-rich domain 3 (CRD3) of DR5, further optionally a DR5 comprising an amino acid sequence as set forth in SEQ ID NO: 62 or SEQ ID NO: 63; and
(b) a second composition comprising, consisting essentially of, or consisting of an effective amount of a second binding agent, wherein the second binding agent selectively binds to a binding partner selected from the group consisting of a human cysteine-rich domain 1 (CRD1), a cysteine-rich domain 2 (CRD2), and a tumor-associated antigen, optionally wherein the second binding agent comprises a DR5 agonist.

25. The method of claim 24, wherein the DR5 biological activity to be activated is present in a cell of a tumor or a cancer, optionally a cell of a solid tumor.

26. The method of claim 23, wherein the first binding agent comprises an antibody, optionally an antibody that is selected from the group consisting of lexatumumab and antibody 1114, wherein antibody 1114 comprises a heavy chain comprising an amino acid sequence as set forth in SEQ ID NO: 26 and a light chain comprising an amino acid sequence as set forth in SEQ ID NO: 27, or a biologically active fragment or homolog thereof, and further wherein antibody 1114 selectively binds an RKCR (SEQ ID NO: 101) tetrapeptide motif present in a human cysteine-rich domain 3 (CRD3) of DR5, optionally a DR5 comprising an amino acid sequence as set forth in SEQ ID NO: 62 or SEQ ID NO: 63.

27. The method of claim 24, wherein the second binding agent that selectively binds to a human CRD1 and/or a human CRD2, optionally wherein the second binding agent comprises a DR5 agonist.

28. The method of claim 27, wherein the DR5 agonist comprises an antibody, optionally an antibody that binds to DR5 CRD1 and/or CRD2, or to an epitope other than a RKCR (SEQ ID NO: 101) tetrapeptide of DR5.

29. The method of claim 24, wherein the first composition and the second composition are provided as a single composition.

30. The method of claim 28, wherein the single composition comprises a bispecific antibody, optionally a 2DEI antibody.

31. The method of claim 29, wherein the single composition comprises a 2DEI antibody, optionally wherein the 2DEI antibody comprises a sequence as set forth in Table 3, optionally any one of SEQ ID NOS: 1-6, or a biologically active fragment and/or homolog thereof.

32. The method of claim 29, wherein the single composition comprises an antibody selected from the group comprising lexatumumab and antibody 1114, wherein antibody 1114 comprises a heavy chain comprising an amino acid sequence as set forth in SEQ ID NO: 26 and a light chain comprising an amino acid sequence as set forth in SEQ ID NO: 27, a biologically active fragment thereof, a homolog thereof, or any combination thereof.

33.-41. (canceled)
